(19) United States
(12) Patent Application Publication
(10) Pub. No.: US 2026/0217744 A1
(43) Pub. Date: Jul. 30, 2026

(54) BRANCHED PHOSPHOLIPIDS

(71) Applicant: BRISTOL-MYERS SQUIBB COMPANY, PRINCETON, NJ (US)

(72) Inventors: JEFFREY O'BRIEN, LA JOLLA, CA (US); SEYEDMEHDI HOSSAINI NASR, SAN DIEGO, CA (US); WILLARD RODNEY FOSS, SAN DIEGO, CA (US); KENDALL CONDON, PHILADELPHIA, PA (US)

(73) Assignee: BRISTOL-MYERS SQUIBB COMPANY, PRINCETON, NJ (US)

(21) Appl. No.: 19/463,247

(22) Filed: Jan. 29, 2026

Related U.S. Application Data
(60) Provisional application No. 63/751,605, filed on Jan. 30, 2025.

(51) Int. Cl. C07F 9/10; A61K 9/51; A61K 31/7105; A61K 38/46; A61K 47/24

(52) U.S. Cl. CPC C07F 9/10; A61K 9/5123; A61K 9/5192; A61K 31/7105; A61K 38/465; A61K 47/24

(57) ABSTRACT

The application is related to compounds of Formula I, their uses and lipid nanoparticle formulations

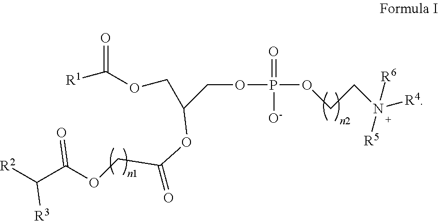

### CROSS REFERENCE TO RELATED APPLICATIONS

This application claims the benefit of U.S. Provisional Application No. 63/751,605, filed Jan. 30, 2025, the entire content of which is hereby incorporated herein by reference.

### FIELD

This invention relates to branched phospholipid compounds and their use in the formation of lipid nanoparticles.

### BACKGROUND OF THE INVENTION

Nucleic acids are useful for the treatment of various diseases and disorders. For example, RNA interference (RNAi) has been the subject of significant research and clinical development. Messenger RNA (mRNA) therapy is an important option for treatment of various diseases, in particular, for those associated with deficiency of one or more proteins.

Small non-coding RNA, which regulates gene expression can be useful for the treatment of variety of diseases and disorders. Based on their biological roles and structures, small non-coding RNAs can be classified into three main categories: miRNAs, siRNAs, and piRNAs. In addition to therapeutic use, gene silencing by siRNA is an important tool to pinpoint the gene responsible for the specific pathological condition. RNA interference tools like siRNA can be used in studying the mammalian cellular signalling pathways. Sequence-specific binding of siRNA to the mRNA, and its site-specific cleavage results in the downregulation or inhibition of the genes responsible for cancer or other pathological conditions. However, there are several hurdles in the use of siRNA and mRNA in therapeutic setting which include degradation by the ribonucleases enzymes, stability of siRNA molecules in physiological conditions, inflammation reactions, site-specific and controlled release of siRNA, and efficient delivery vehicle. These barriers need to be overcome for the success of the use of mRNA and siRNA as therapeutics. Nitin Bharat Charbe, et. al., Small interfering RNA for cancer treatment: overcoming hurdles in delivery, Acta Pharmaceutica Sinica B, 10 (11) 2020, 2075-2109.

### SUMMARY

The present disclosure provides branched phospholipid compounds having the structure of Formula I:

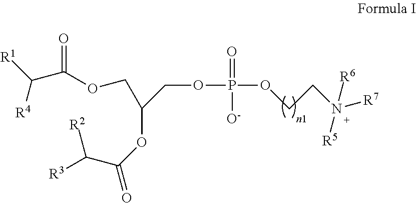

or a pharmaceutically acceptable salt thereof,
Wherein n1 is an integer selected from 0, 1 or 2;
Each R¹, R², R³ and R⁴ is independently selected from C₄₋₂₀ alkyl and C₄₋₂₀ alkenyl;
wherein said R¹, R², R³ and R⁴ is optionally substituted with one or two —O(C(O))—C₄₋₁₆ alkyl, —O(C(O))—C₄₋₁₆ alkenyl or —O(C(O))—C₄₋₁₆ alkynyl;
Each R⁵, R⁶ and R⁷ is independently selected from H and C₁₋₃ alkyl.

The application further provides pharmaceutical compositions comprising one or more lipids of Formula I. The formulations can be used for the delivery of one or more nucleic acid therapeutic agents, for example mRNA, siRNA or miRNA. The compositions can optionally include additional lipids, such as non-cationic lipids, PEG-modified lipids and optionally cholesterol.

### DETAILED DESCRIPTION

In one aspect, the present disclosure provides lipid compounds having the structure of Formula I:

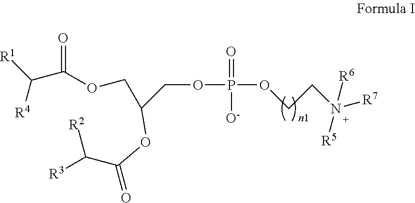

Wherein n1 is an integer selected from 0, 1 or 2;
Each R¹, R², R³ and R⁴ is independently selected from C₄₋₂₀ alkyl and C₄₋₂₀ alkenyl;
wherein said R¹, R², R³ and R⁴ is optionally substituted with one or two —O(C(O))—C₄₋₁₆ alkyl, —O(C(O))—C₄₋₁₆ alkenyl or —O(C(O))—C₄₋₁₆ alkynyl;
Each R⁵, R⁶ and R⁷ is independently selected from H and C₁₋₃ alkyl.

In one embodiment, provided are compounds of Formula II:

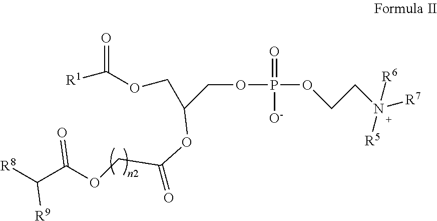

or a pharmaceutically acceptable salt thereof;
wherein, each R⁸ and R⁹ is independently selected from C₄₋₁₀ alkyl and C₄₋₁₀ alkenyl; and,
n2 is an integer selected from 4, 5, 6, 7 or 8.

In one embodiment, provided are compounds of Formula III:

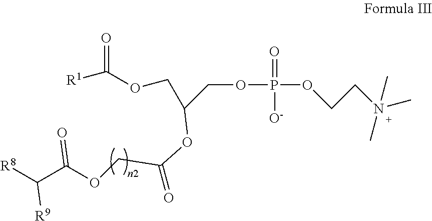

or a pharmaceutically acceptable salt thereof.

In one embodiment, provided are compounds of Formula IV:

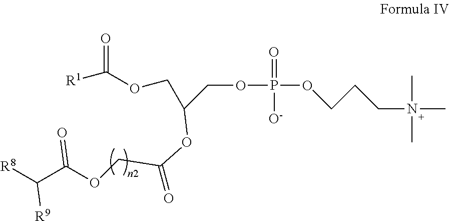

or a pharmaceutically acceptable salt thereof.

In one embodiment, provided are compounds of Formula V:

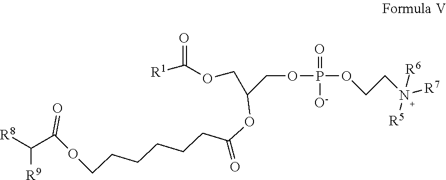

or a pharmaceutically acceptable salt thereof.

In one embodiment, provided are compounds of Formula VI:

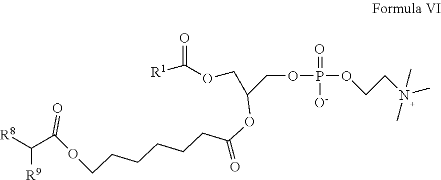

or a pharmaceutically acceptable salt thereof.

In a preferred embodiment, provided are compounds of Formula I-VI, wherein each R¹ and R⁴ is interpedently selected from C₁₆₋₂₀ alkyl, C₁₆₋₂₀ alkenyl, preferably C₁₇ alkyl or C₁₇ alkenyl.

In a preferred embodiment, provided are compounds of Formula I-VI, wherein each R² and R³ is independently selected from C₆₋₁₂ alkyl and C₆₋₁₂ alkenyl, preferably C₉ alkenyl and C₉ alkyl.

In a preferred embodiment, each R¹, R², R³ and R⁴ is independently selected from:

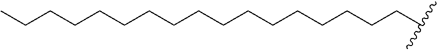

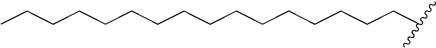

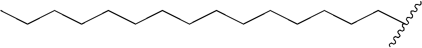

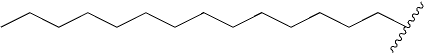

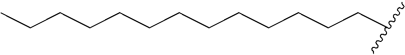

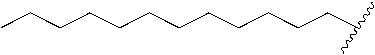

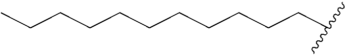

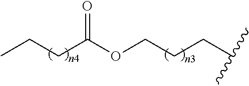

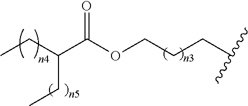

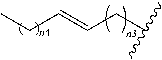

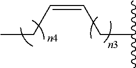

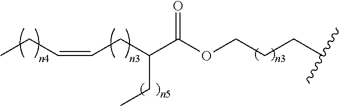

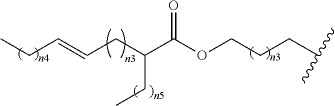

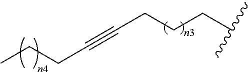

Wherein each n3, n4 and n5 is an integer selected from 2, 3, 4, 5, 6, 7, 8, 9, 10, 11 and 12.

In one aspect, provided is a method for encapsulating mRNA in lipid nanoparticles comprising the steps of mixing one or more lipids of Formula I, and optionally one or more additional lipids in a lipid solution with one or more mRNAs in an mRNA solution to form mRNA encapsulated within the LNP.

The following are definitions of terms used in this specification and appended claims. The initial definition provided for a group or term herein applies to that group or term throughout the specification and claims, individually or as part of another group, unless otherwise indicated.

In some embodiments, provided are lipid nanoparticle formulations comprising lipids of Formula I and one or more mRNA moieties. In some embodiments, the mRNA is a modified mRNA. Modifications, for example, can improve resistance to nuclease digestion in vivo. As used herein, the terms “modification” and “modified” as such terms relate to the nucleic acids provided herein, include at least one alteration which preferably enhances stability and renders the mRNA more stable (e.g., resistant to nuclease digestion) than the wild-type or naturally occurring version of the mRNA. As used herein, the terms “stable” and “stability” as such terms relate to the nucleic acids of the present invention, and particularly with respect to the mRNA, refer to increased or enhanced resistance to degradation by, for example nucleases (i.e., endonucleases or exonucleases) which are normally capable of degrading such mRNA. Also contemplated by the terms “modification” and “modified” as such terms related to the mRNA of the present invention are alterations which improve or enhance translation of mRNA nucleic acids, including for example, the inclusion of sequences which function in the initiation of protein translation (e.g., the Kozac consensus sequence). (Kozak, M., Nucleic Acids Res 15 (20): 8125-48 (1987)).

In one embodiment, provided are lipid nanoparticle compositions prepared to optimize delivery of the mRNA to a target cell. For example, if the target cell is a hepatocyte the properties of the lipid nanoparticles may be optimized to effectively deliver such transfer vehicle to the target cell, reduce immune clearance and/or promote retention in that target cell. In one embodiment, the compositions of the present invention may be combined with agents that facilitate the transfer of exogenous mRNA (e.g., agents which disrupt or improve the permeability of the blood brain barrier and thereby enhance the transfer of exogenous mRNA to the target cells).

The process of incorporation of a desired entity (e.g., a nucleic acid) into a lipid nanoparticle is often referred to as “loading” (Lasic, et al., FEBS Lett., 312: 255-258, 1992). The LNP-incorporated nucleic acids may be completely or partially located in the interior space of the LNP, within the bilayer membrane of the LNP, or associated with the exterior surface of the LNP membrane. The purpose of incorporating a mRNA into a lipid nanoparticle is often to protect the nucleic acid from an environment which may contain enzymes or chemicals that degrade nucleic acids and/or systems or receptors that cause the rapid excretion of the nucleic acids. Accordingly, in a preferred embodiment of the present invention, the selected transfer vehicle is capable of enhancing the stability of the mRNA contained therein. The liposome can allow the encapsulated mRNA to reach the target cell and/or may preferentially allow the encapsulated mRNA to reach the target cell, or alternatively limit the delivery of such mRNA to other sites or cells where the presence of the administered mRNA may be useless or undesirable. Furthermore, incorporating the mRNA into a transfer vehicle, such as for example, a cationic liposome, also facilitates the delivery of such mRNA into a target cell.

Ideally, liposomal transfer vehicles are prepared to encapsulate one or more desired mRNA such that the compositions demonstrate a high transfection efficiency and enhanced stability. While liposomes can facilitate introduction of nucleic acids into target cells, the addition of polycations (e.g., poly L-lysine and protamine), as a copolymer can facilitate, and in some instances markedly enhance the transfection efficiency of several types of cationic liposomes by 2-28 fold in a number of cell lines both in vitro and in vivo. (See N.J. Caplen, et al., Gene Ther. 1995; 2: 603; S. Li, et al., Gene Ther. 1997; 4, 891.)

### Lipid Nanoparticles

In a preferred embodiment of the present invention, the transfer vehicle is formulated as a lipid nanoparticle. As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more mRNA to one or more target cells. Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a mRNA to a target cell.

As used in the present specification, the following words, phrases and symbols are generally intended to have the meanings as set forth below, except to the extent that the context in which they are used indicates otherwise.

Compounds of this invention may have one or more asymmetric centers. Unless otherwise indicated, all chiral (enantiomeric and diastereomeric) and racemic forms of compounds of the present invention are included in the present invention. Many geometric isomers of olefins, C≡N double bonds, and the like can also be present in the compounds, and all such stable isomers are contemplated in the present invention. Cis- and trans-geometric isomers of the compounds of the present invention are described and may be isolated as a mixture of isomers or as separated isomeric forms. The present compounds can be isolated in optically active or racemic forms. It is well known in the art how to prepare optically active forms, such as by resolution of racemic forms or by synthesis from optically active starting materials. All chiral, (enantiomeric and diastereomeric) and racemic forms and all geometric isomeric forms of a structure are intended, unless the specific stereochemistry or isomer form is specifically indicated.

When any variable (e.g., R³) occurs more than one time in any constituent or formula for a compound, its definition at each occurrence is independent of its definition at every other occurrence. Thus, for example, if a group is shown to be substituted with 0-2 R³, then said group may optionally be substituted with up to two R³ groups and R³ at each occurrence is selected independently from the definition of R³. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are nitrogen atoms (e.g., amines) on compounds of the present invention, these can be converted to N-oxides by treatment with an oxidizing agent (e.g., MCPBA and/or hydrogen peroxides) to afford other compounds of this invention. Thus, all shown and claimed nitrogen atoms are considered to cover both the shown nitrogen and its N-oxide (N→O) derivative.

In accordance with a convention used in the art,

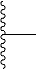

is used in structural formulas herein to depict the bond that is the point of attachment of the moiety or substituent to the core or backbone structure.

A dash “-” that is not between two letters or symbols is used to indicate a point of attachment for a substituent. For example, —CONH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The term “optionally substituted” in reference to a particular moiety of the compound of Formula I (e.g., an optionally substituted heteroaryl group) refers to a moiety having 0, 1, 2, or more substituents. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “at least one chemical entity” is interchangeable with the term “a compound”.

The prefix “Cᵤ₋ᵥ” indicates that the following group has from u to v carbon atoms. For example, “C₁₋₆ alkyl” indicates that the alkyl group has from 1 to 6 carbon atoms.

As used herein, the term “alkyl” or “alkylene” is intended to include both branched and straight-chain saturated aliphatic hydrocarbon groups having the specified number of carbon atoms. For example, “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁-C₆ alkyl” denotes alkyl having 1 to 6 carbon atoms. Alkyl groups can be unsubstituted or substituted so that one or more of its hydrogens are replaced by another chemical group. Example alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

“Alkenyl” or “alkenylene” is intended to include hydrocarbon chains of either straight or branched configuration and having one or more double carbon-carbon bonds that may occur in any stable point along the chain. For example, “C₂₋₆ alkenyl” (or alkenylene), is intended to include C₂, C₃, C₄, C₅, and C₆ alkenyl groups. Examples of alkenyl include, but are not limited to, ethenyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, and the like.

“Alkynyl” or “alkynylene” is intended to include hydrocarbon chains of either straight or branched configuration and having one or more triple carbon-carbon bonds that may occur in any stable point along the chain. For example, “C₂₋₆ alkynyl” (or alkynylene), is intended to include C₂, C₃, C₄, C₅, and C₆ alkynyl groups; such as ethynyl, propynyl, butynyl, pentynyl, hexynyl and the like.

One skilled in the field will understand that, when the designation “CO₂” is used herein, this is intended to refer to the group

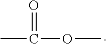

When the term “alkyl” is used together with another group, such as in “arylalkyl”, this conjunction defines with more specificity at least one of the substituents that the substituted alkyl will contain. For example, “arylalkyl” refers to a substituted alkyl group as defined above where at least one of the substituents is an aryl, such as benzyl. Thus, the term aryl(C₀₋₄)alkyl includes a substituted lower alkyl having at least one aryl substituent and also includes an aryl directly bonded to another group, i.e., aryl(Co)alkyl. The term “heteroarylalkyl” refers to a substituted alkyl group as defined above where at least one of the substituents is a heteroaryl.

When reference is made to a substituted alkenyl, alkynyl, alkylene, alkenylene, or alkynylene group, these groups are substituted with one to three substituents as defined above for substituted alkyl groups.

The term “alkoxy” refers to an oxygen atom substituted by alkyl or substituted alkyl, as defined herein. For example, the term “alkoxy” includes the group —O—C₁₋₆alkyl such as methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. “Lower alkoxy” refers to alkoxy groups having one to four carbons.

The term “cycloalkyl” refers to cyclized alkyl groups, including mono-, bi- or poly-cyclic ring systems. C₃₋₇ cycloalkyl is intended to include C₃, C₄, C₅, C₆, and C₇ cycloalkyl groups. Example cycloalkyl groups include, but are not limited to, cyclopropyl, cyclobutyl, cyclopentyl, cyclohexyl, norbornyl, and the like. As used herein, “carbocycle” or “carbocyclic residue” is intended to mean any stable 3-, 4-, 5-, 6-, or 7-membered monocyclic or bicyclic or 7-, 8-, 9-, 10-, 11-, 12-, or 13-membered bicyclic or tricyclic ring, any of which may be saturated, partially unsaturated, unsaturated or aromatic. Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocycle (e.g., [2.2.2]bicyclooctane). Preferred carbocycles, unless otherwise specified, are cyclopropyl, cyclobutyl, cyclopentyl, cyclohexyl, and phenyl. When the term “carbocycle” is used, it is intended to include “aryl”. A bridged ring occurs when one or more carbon atoms link two non-adjacent carbon atoms. Preferred bridges are one or two carbon atoms. It is noted that a bridge always converts a monocyclic ring into a bicyclic ring. When a ring is bridged, the substituents recited for the ring may also be present on the bridge.

The term “aryl” refers to monocyclic or bicyclic aromatic hydrocarbon groups having 6 to 12 carbon atoms in the ring portion, such as phenyl, and naphthyl groups, each of which may be substituted.

Accordingly, in compounds of formula I, the term “cycloalkyl” includes cyclopropyl, cyclobutyl, cyclopentyl, cyclohexyl, cycloheptyl, bicyclooctyl, etc.

The term “halo” or “halogen” refers to chloro, bromo, fluoro and iodo.

The term “haloalkyl” means a substituted alkyl having one or more halo substituents. For example, “haloalkyl” includes mono, bi, and trifluoromethyl.

The term “haloalkoxy” means an alkoxy group having one or more halo substituents. For example, “haloalkoxy” includes OCF₃.

The terms “heterocycle”, “heterocycloalkyl”, “heterocyclo”, “heterocyclic”, or “heterocyclyl” may be used interchangeably and refer to substituted and unsubstituted 3- to 7-membered monocyclic groups, 7- to 11-membered bicyclic groups, and 10- to 15-membered tricyclic groups, in which at least one of the rings has at least one heteroatom (O, S or N), said heteroatom containing ring preferably having 1, 2, or 3 heteroatoms selected from O, S, and N. Each ring of such a group containing a heteroatom can contain one or two oxygen or sulfur atoms and/or from one to four nitrogen atoms provided that the total number of heteroatoms in each ring is four or less, and further provided that the ring contains at least one carbon atom. The nitrogen and sulfur atoms may optionally be oxidized and the nitrogen atoms may optionally be quaternized. The fused rings completing the bicyclic and tricyclic groups may contain only carbon atoms and may be saturated, partially saturated, or fully unsaturated. The heterocyclo group may be attached at any available nitrogen or carbon atom. As used herein the terms “heterocycle”, “heterocycloalkyl”, “heterocyclo”, “heterocyclic”, and “heterocyclyl” include “heteroaryl” groups, as defined below.

In addition to the heteroaryl groups described below, exemplary monocyclic heterocyclyl groups include azetidinyl, pyrrolidinyl, oxetanyl, imidazolinyl, oxazolidinyl, isoxazolinyl, thiazolidinyl, isothiazolidinyl, tetrahydrofuranyl, piperidyl, piperazinyl, 2-oxopiperazinyl, 2-oxopiperidyl, 2-oxopyrrolodinyl, 2-oxoazepinyl, azepinyl, 1-pyridonyl, 4-piperidonyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl and the like. Exemplary bicyclic heterocyclo groups include quinuclidinyl.

The term “heteroaryl” refers to substituted and unsubstituted aromatic 5- or 6-membered monocyclic groups, 9- or 10-membered bicyclic groups, and 11- to 14-membered tricyclic groups which have at least one heteroatom (O, S or N) in at least one of the rings, said heteroatom-containing ring preferably having 1, 2, or 3 heteroatoms selected from O, S, and N. Each ring of the heteroaryl group containing a heteroatom can contain one or two oxygen or sulfur atoms and/or from one to four nitrogen atoms provided that the total number of heteroatoms in each ring is four or less and each ring has at least one carbon atom. The fused rings completing the bicyclic and tricyclic groups may contain only carbon atoms and may be saturated, partially saturated, or unsaturated. The nitrogen and sulfur atoms may optionally be oxidized and the nitrogen atoms may optionally be quaternized. Heteroaryl groups which are bicyclic or tricyclic must include at least one fully aromatic ring but the other fused ring or rings may be aromatic or non-aromatic. The heteroaryl group may be attached at any available nitrogen or carbon atom of any ring. As valence allows, if said further ring is cycloalkyl or heterocyclo it is additionally optionally substituted with ═O(oxo).

Exemplary monocyclic heteroaryl groups include pyrrolyl, pyrazolyl, pyrazolinyl, imidazolyl, oxazolyl, isoxazolyl, thiazolyl, thiadiazolyl, isothiazolyl, furanyl, thienyl, oxadiazolyl, pyridyl, pyrazinyl, pyrimidinyl, pyridazinyl, triazinyl and the like.

Exemplary bicyclic heteroaryl groups include indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridyl, furopyridyl, dihydroisoindolyl, tetrahydroquinolinyl and the like.

Exemplary tricyclic heteroaryl groups include carbazolyl, benzindolyl, phenanthrollinyl, acridinyl, phenanthridinyl, xanthenyl and the like.

Unless otherwise indicated, when reference is made to a specifically-named aryl (e.g., phenyl), cycloalkyl (e.g., cyclohexyl), heterocyclo (e.g., pyrrolidinyl, piperidinyl, and morpholinyl) or heteroaryl (e.g., tetrazolyl, imidazolyl, pyrazolyl, triazolyl, thiazolyl, and furyl) the reference is intended to include rings having 0 to 3, preferably 0 to 2, substituents selected from those recited above for the aryl, cycloalkyl, heterocyclo and/or heteroaryl groups, as appropriate.

The term “carbocyclyl” or “carbocyclic” refers to a saturated or unsaturated monocyclic or bicyclic ring in which all atoms of all rings are carbon. Thus, the term includes cycloalkyl and aryl rings. Monocyclic carbocycles have 3 to 6 ring atoms, still more typically 5 or 6 ring atoms. Bicyclic carbocycles have 7 to 12 ring atoms, e.g., arranged as a bicyclo[4,5], [5,5], [5,6] or [6,6]system, or 9 or 10 ring atoms arranged as a bicyclo[5,6] or [6,6]system. Examples of mono- and bicyclic carbocycles include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, phenyl and naphthyl. The carbocyclic ring may be substituted in which case the substituents are selected from those recited above for cycloalkyl and aryl groups.

The term “alkylthio” refers to the group “alkyl-S—”.

The term “acyl” refers to a group —C(O)R, wherein R is hydrogen, alkyl, cycloalkyl, heterocyclyl, aryl, heteroalkyl, or heteroaryl; each of which may be optionally substituted, as defined herein. Examples of acyl include formyl, acetyl, cylcohexylcarbonyl, cyclohexylmethyl-carbonyl, and benzoyl.

The term “amido” refers to both a “C-amido” group which refers to the group —C(O)NR^{g}R^{h} and an “N-amido” group which refers to the group —NR^{g}C(O)R^{h}, wherein R^{g} and R^{h} are independently selected from hydrogen, alkyl, aryl, haloalkyl, or heteroaryl; each of which may be optionally substituted.

The term “amino” refers to the group —NR^{g}R^{h} wherein R^{g} and R^{h} are independently selected from hydrogen, alkyl, haloalkyl, aryl, or heteroaryl; each of which may be optionally substituted.

The term “azido” refers to —N₃.

The term “carbamoyl” refers to both an “O-carbamoyl” group which refers to the group —O—C(O)NRⁱR^{j} and an “N-carbamoyl” group which refers to the group —NRⁱC(O)OR^{j}, wherein Rⁱ and R^{j} are independently selected from hydrogen, alkyl, aryl, haloalkyl, or heteroaryl; each of which may be optionally substituted.

The term “carboxyl” refers to —C(O)OH.

The term “carboxyl ester” refers to both —OC(O)R and —C(O)OR^{g}, wherein R^{g} is hydrogen, alkyl, cycloalkyl, heterocyclyl, aryl, heteroalkyl, or heteroaryl; each of which may be optionally substituted, as defined herein.

The term “cyano” or “carbonitrile” refers to the group —CN.

The term “cycloalkyl” refers to a saturated or partially unsaturated cyclic alkyl group having a single ring or multiple rings including fused, bridged, and spiro ring systems. The term “cycloalkyl” includes cycloalkenyl groups (i.e. the cyclic group having at least one double bond). As used herein, cycloalkyl has from 3 to 20 ring carbon atoms (i.e., C.sub.3-20 cycloalkyl), 3 to 12 ring carbon atoms (i.e., C.sub.3-12 cycloalkyl), 3 to 10 ring carbon atoms (i.e., C.sub.3-10 cycloalkyl), 3 to 8 ring carbon atoms (i.e., C.sub.3-8 cycloalkyl), or 3 to 6 ring carbon atoms (i.e., C.sub.3-6 cycloalkyl). Examples of cycloalkyl groups include cyclopropyl, cyclobutyl, cyclopentyl, and cyclohexyl.

The term “heteroatoms” shall include oxygen, sulfur and nitrogen.

When the term “unsaturated” is used herein to refer to a ring or group, the ring or group may be fully unsaturated or partially unsaturated.

Throughout the specification, groups and substituents thereof may be chosen by one skilled in the field to provide stable moieties and compounds and compounds useful as pharmaceutically-acceptable compounds and/or intermediate compounds useful in making pharmaceutically-acceptable compounds.

It should be understood that the selections for all groups, including for example, alkoxy, thioalkyl, and aminoalkyl, will be made by one skilled in the field to provide stable compounds.

The term “substituted”, as used herein, means that any one or more hydrogens on the designated atom or group is replaced with a selection from the indicated group, provided that the designated atom's normal valence is not exceeded. When a substituent is oxo, or keto, (i.e., ═O) then 2 hydrogens on the atom are replaced. Keto substituents are not present on aromatic moieties. Unless otherwise specified, substituents are named into the core structure. For example, it is to be understood that when (cycloalkyl)alkyl is listed as a possible substituent, the point of attachment of this substituent to the core structure is in the alkyl portion. Ring double bonds, as used herein, are double bonds that are formed between two adjacent ring atoms (e.g., C═C, C═N, or N═N).

Combinations of substituents and/or variables are permissible only if such combinations result in stable compounds or useful synthetic intermediates. A stable compound or stable structure is meant to imply a compound that is sufficiently robust to survive isolation from a reaction mixture to a useful degree of purity, and subsequent formulation into an efficacious therapeutic agent. It is preferred that the presently recited compounds do not contain a N-halo, S(O)₂H, or S(O)H group.

Unless otherwise indicated, reference to an inventive compound is understood to include reference to the free form and to salts thereof. The term “salt(s)” denotes acidic and/or basic salts formed with inorganic and/or organic acids and bases. In addition, the term “salt(s)” may include zwitterions (inner salts), e.g., when a compound of formula I, contains both a basic moiety, such as an amine or a pyridine or imidazole ring, and an acidic moiety, such as a carboxylic acid. Pharmaceutically acceptable (i.e., non-toxic, physiologically acceptable) salts are preferred, such as, for example, acceptable metal and amine salts in which the cation does not contribute significantly to the toxicity or biological activity of the salt. However, other salts may be useful, e.g., in isolation or purification steps which may be employed during preparation, and thus, are contemplated within the scope of the invention. Salts of the compounds of herein may be formed, for example, by reacting a compound of the Formula I, II or III with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts include ammonium salts, alkali metal salts such as sodium, lithium, and potassium salts; alkaline earth metal salts such as calcium and magnesium salts; barium, zinc, and aluminum salts; salts with organic bases (for example, organic amines) such as trialkylamines such as triethylamine, procaine, dibenzylamine, N-benzyl-β-phenethylamine, 1-ephenamine, N,N′-dibenzylethylene-diamine, dehydroabietylamine, N-ethylpiperidine, benzylamine, dicyclohexylamine or similar pharmaceutically acceptable amines and salts with amino acids such as arginine, lysine and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Preferred salts include monohydrochloride, hydrogensulfate, methanesulfonate, phosphate or nitrate salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically-acceptable salts include, but are not limited to, mineral or organic acid salts of basic groups such as amines; and alkali or organic salts of acidic groups such as carboxylic acids. The pharmaceutically-acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th Edition, Mack Publishing Company, Easton, PA (1990), the disclosure of which is hereby incorporated by reference.

“Stable compound” and “stable structure” are meant to indicate a compound that is sufficiently robust to survive isolation to a useful degree of purity from a reaction mixture, and formulation into an efficacious therapeutic agent. The present invention is intended to embody stable compounds.

“Therapeutically effective amount” is intended to include an amount of a compound of the present invention alone or an amount of the combination of compounds claimed or an amount of a compound of the present invention in combination with other active ingredients effective to act as an inhibitor of USP1, or effective to treat or prevent proliferative disorders, such as cancer.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting its development; and/or (c) relieving the disease-state, i.e., causing regression of the disease state.

All stereoisomers of the compounds of the instant invention are contemplated, either in admixture or in pure or substantially pure form. Stereoisomers may include compounds which are optical isomers through possession of one or more chiral atoms, as well as compounds which are optical isomers by virtue of limited rotation about one or more bonds (atropisomers). The definition of compounds according to the invention embraces all the possible stereoisomers and their mixtures. It very particularly embraces the racemic forms and the isolated optical isomers having the specified activity. The racemic forms can be resolved by physical methods, such as, for example, fractional crystallization, separation or crystallization of diastereomeric derivatives or separation by chiral column chromatography. The individual optical isomers can be obtained from the racemates from the conventional methods, such as, for example, salt formation with an optically active acid followed by crystallization.

The present invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed.

### Methods of Preparation

### EXAMPLES

The methods and conditions used in these examples, and the actual compounds prepared in these Examples, are not meant to be limiting, but are meant to demonstrate how the compounds can be prepared. Starting materials and reagents used in these examples, when not prepared by a procedure described herein, are generally either commercially available, or are reported in the chemical literature, or may be prepared by using procedures described in the chemical literature.

ABBREVIATIONS

| | |
|---|---|
| Ac | Acetyl |
| ACN | Acetonitrile |
| anhyd. | Anhydrous |
| aq. | Aqueous |
| Bn | Benzyl |
| Boc-anhydride | di-tert-butyl decarbonate |
| Bu | Butyl |
| Boc | tert-butoxycarbonyl |
| CV | Column Volumes |
| DCE | Dichloroethane |
| DCM | Dichloromethane |
| DMAP | Dimethylaminopyridine |
| DMF | Dimethylformamide |
| DMSO | Dimethylsulfoxide |
| EtOAc | ethyl acetate |
| Et | Ethyl |
| Et₃N | Triethylamine |
| H or H₂ | Hydrogen |
| h, hr or hrs | hour(s) |
| hex | Hexane |
| i | Iso |
| HCl | hydrochloric acid |
| HPLC | high pressure liquid chromatography |
| LC | liquid chromatography |
| LCMS | liquid chromatography-mass spectroscopy |
| LiAlH₄ | lithium aluminum hydride |
| M | Molar |
| mM | Millimolar |
| Me | Methyl |
| MeOH | Methanol |
| MHz | Megahertz |
| min. | minute(s) |
| mins | minute(s) |
| M⁺¹ | (M + H)⁺ |
| MS | mass spectrometry |
| n or N | Normal |
| NBS | n-bromosuccinimide |
| NCS | n-chlorosuccinimide |
| nm | Nanometer |
| nM | Nanomolar |
| NMP | N-methylpyrrolidinone |
| Pd/C | palladium on carbon |
| PdCl₂(dppf) | [1,1′- |
| | bis(diphenylphosphino)ferrocene]dichloropalladium(II) |
| Ph | Phenyl |
| Pr | Propyl |
| PSI | pounds per square inch |
| Ret Time | retention time |
| sat. | saturated |
| SFC | supercritical fluid chromatography |
| TEA | triethylamine |
| TFA | trifluoroacetic acid |
| THF | tetrahydrofuran |
| XPhos Precatalyst | chloro(2-dicyclohexylphosphino- |
| | 2′,4′,6′-triisopropyl-1,1′- |
| | biphenyl)[2-(2′-amino-1,1′- |
| | biphenyl)]palladium(II) |

### Methods of Preparation

### EXAMPLES

The methods and conditions used in these examples, and the actual compounds prepared in these Examples, are not meant to be limiting, but are meant to demonstrate how the compounds can be prepared. Starting materials and reagents used in these examples, when not prepared by a procedure described herein, are generally either commercially available, or are reported in the chemical literature, or may be prepared by using procedures described in the chemical literature.

### Example 1: Synthesis of Lipid-1

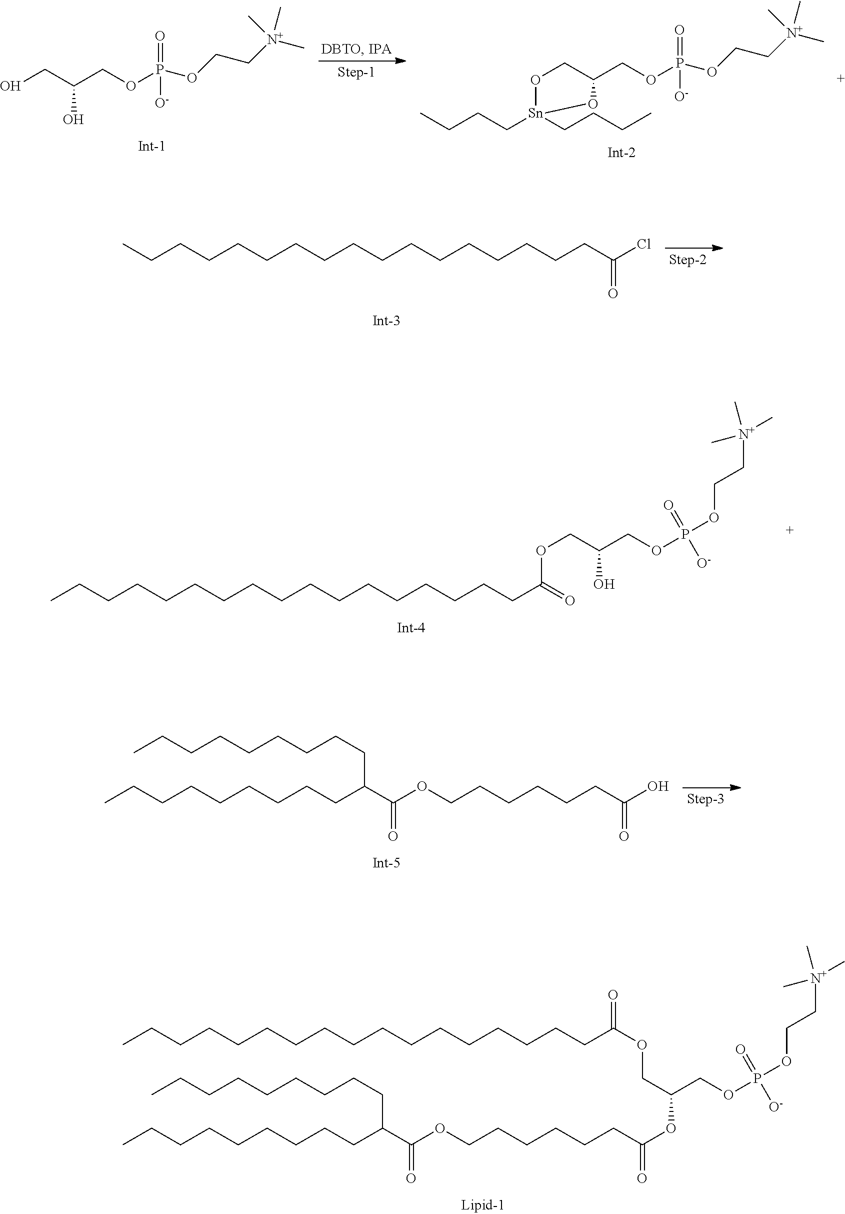

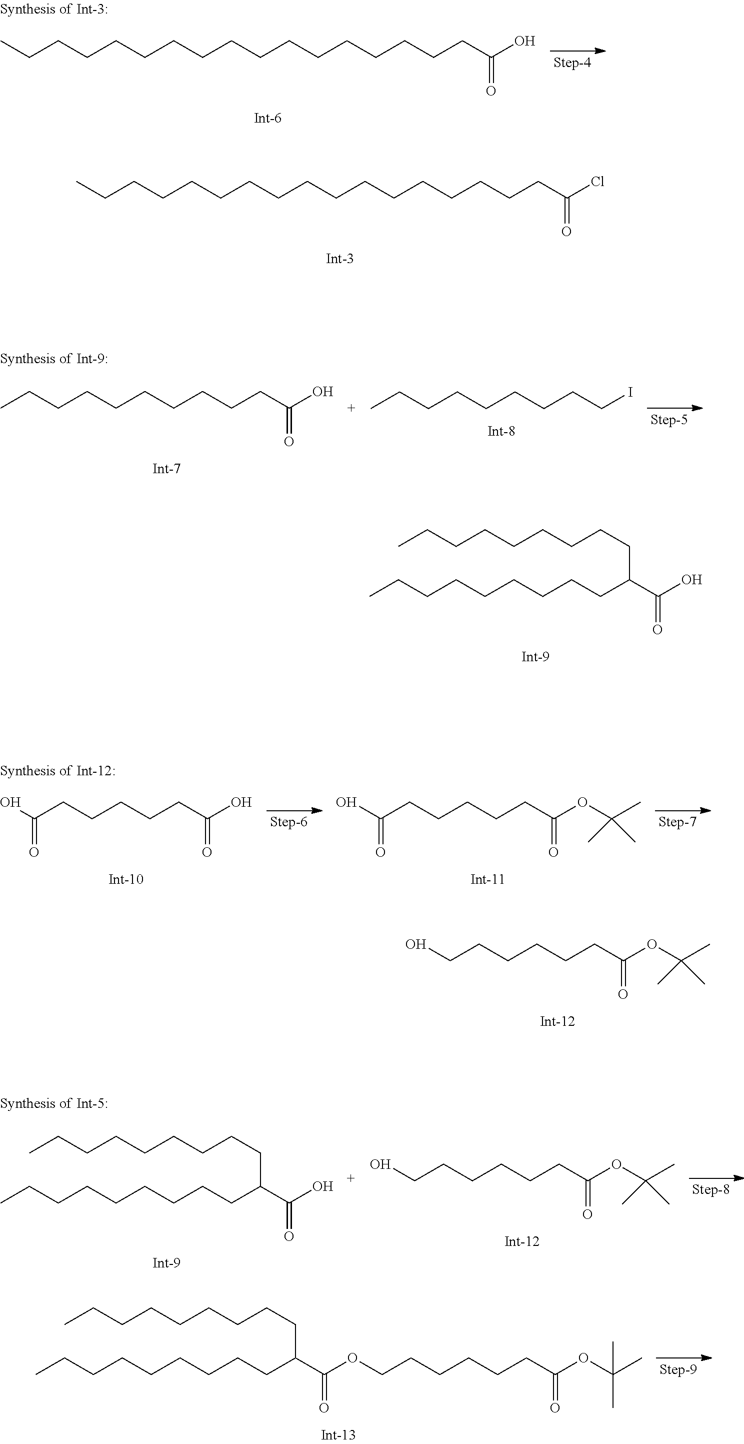

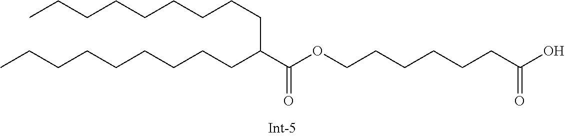 [SMILES: CCCCCCCCCC(CCCCCCCCC)C(=O)OCCCCCCC(=O)O]

### Synthetic Procedure

### Step-1&2: Synthesis of (2-{[(2R)-2-hydroxy-3-(octadecanoyloxy)propyl phosphonato]oxy}ethyl)trimethylazanium (Int-4)

Choline glycerophosphate (5 g, 0.0194 mol) and dibutyltin oxide (DBTO) (5.3 g, 0.0213 mol) were suspended in 2-propanol (250 mL) and refluxed for 18 h. The mixture was cooled to 0 to 5° C., TEA (3.3 mL, 0.0233 mol) and Stearoyl chloride (Int-3) (7.06 g, 0.0233 mol) were added. Resulting reaction mixture was stirred over a period of 18 h at 25-30° C. under argon atmosphere. Progress of the reaction was monitored by TLC. TLC shows new polar spot formation.

TLC eluent: (CH₂Cl₂/MeOH/H₂O: 65/25/4)-KMnO₄ active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Crude residue was dissolved in ethanol (100 mL) and precipitated with acetone (400 mL) at −10° C. to afford (2-{[(2R)-2-hydroxy-3-(octadecanoyloxy)propylphosphonato]oxy}ethyl)trimethylazanium (Int-4) as a white solid.

Yield: 6 g, 59.4%; Characterized by ¹H-NMR

### Step-3: Synthesis of Trimethyl(2-{[(2R)-2-({7-[(2-nonylundecanoyl)oxy]heptanoyl}oxy)-3-(octadecanoyloxy)propyl phosphonato]oxy}ethyl)azanium (Lipid-1)

To a solution of Int-4 (1.5 g, 0.0028 mol) in DCM (100 mL, 50 V) at 0 to 5° C., were added Int-5 (1.8 g, 0.0043 mol), 2-methyl-6-nitrobenzoic anhydride [MNBA](4.9 g, 0.0143 mol) and DMAP (3.4 g, 0.028 mol). Resulting reaction mixture was stirred for 48 h at 25-30° C. Progress of the reaction was monitored by TLC. TLC shows new nonpolar spot formation.

TLC eluent: (IPA/EtOAc/H₂O: 8/5/4) Molybdenum blue reagent

Work up: Reaction mixture was directly concentrated under reduced pressure at 40° C. Residue was dissolved in DCM and DOWEX H⁺ (20% w/w) resin was added, stirred gently for 1 h at 25-30° C. After 1 h, TLC shows absence of DMAP and then DCM layer was filtered and concentrated under reduced pressure at 40° C. Crude was purified manually using Neutral alumina column chromatography using CH₂Cl₂:MeOH:H₂O (65/25/4) to afford Lipid-1 as brown semi-solid.

Yield: 560 mg, 20.74%; Characterized by ¹H-NMR, ³¹P-NMR and LCMS

LCMS: (EI, m/z) calcd for C₅₃H₁₀₄NO₁₀P [M+H]: 947.0; HPLC (CAD) Purity: 95.08%

¹H NMR (400 MHz, MeOD): 5.27-5.22 (1H, m), 4.46-4.42 (1H, dd, J=12 Hz), 4.27 (2H, m), 4.17-4.14 (1H, m), 4.08 (2H, t, J=6.4 Hz), 4.0 (2H, t, J=5.6 Hz), 3.65-3.63 (2H, m), 3.22 (9H, s), 2.37-2.30 (5H, m), 1.66-1.58 (8H, m), 1.47-1.44 (6H, m), 1.44-1.29 (56H, m), 0.9 (9H, t, J=7.2 Hz);

### Step-4: Synthesis of Octadecanoyl Chloride (Int-3)

A stirred solution of Stearic acid Int-6 (15 g, 0.052 mol) in DCM (150 mL, 10 V) was cooled to 0-10° C., Oxalyl chloride (9 mL, 0.1054 mol) was added in a dropwise manner at 0-5° C. and catalytic DMF (0.1 mL) was added. Resulting reaction was stirred for 5 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 40° C. to dryness to afford Int-3 as yellow liquid. Crude was taken as such for next step immediately.

Yield: 12 g (crude)

### Step-5: Synthesis of 2-nonylundecanoic Acid (Int-9)

A solution of Undecanoic acid (Int-7) (10 g, 0.0536 mol) in THF (300 mL, 30 V) was cooled to −5 to 0° C., NaH (60% in oil) (2.1 g, 0.0644 mol) was added in a dropwise manner by maintaining same temperature, stirred for 30 min. To the above reaction mixture LDA-2M in THF (59 mL, 0.1179 mol) was added in a drop wise manner by maintaining temperature −5 to 0° C., stirred for 30 min. 1-Iodononane (Int-8) (16.3 g, 0.0644 mol) was added at 0-10° C. The resultant reaction mixture was warmed to room temperature and refluxed at 45° C., 18 h. Progress of the reaction was monitored by TLC. TLC shows non-polar spot formation along with SM.

TLC eluent: 20% in EtOAc in Hexane

Work up: Reaction mass was cooled to −5 to 0° C., quenched with the addition of 1.5 N HCl by maintaining −5 to 0° C. The resultant reaction mixture was warmed to room temperature and extracted with ethyl acetate (1000 mL) and washed with purified water (800 mL). Organic layer was dried over anhydrous sodium sulphate and concentrated under reduced pressure at 45° C. to afford yellow liquid. crude was purified through combiflash at 3-4% ethyl acetate-hexane to afford Int-9 as pale-yellow solid.

Yield: 8.2 g, 49.1%; Characterized by ¹H-NMR

### Step-6: Synthesis of 7-(Tert-butoxy)-7-oxoheptanoic Acid (Int-11)

To a stirred solution of Pimelic acid (Int-10) (20 g, 0.1248 mol) in THF (200 mL, 10 V) and tert-butanol (100 mL, 5 V) at 0° C., Boc anhydride (28.6 mL, 0.1248 mol) was added dropwise followed by DMAP (7.6 g, 0.0624 mol) in a dropwise manner and slowly brought to ambient temperature. Resulting reaction was stirred for 18 h at 25-30° C. Progress of the reaction was monitored by TLC. TLC shows new non-polar spot formation.

TLC eluent: 10% MeOH in DCM (KMnO4 active)

Work up: Reaction mixture was directly concentrated under reduced pressure at 45° C. Crude was purified through combiflash at 7-8% MeOH/DCM to afford Int-11 as oily liquid.

Yield: 9.5 g, 35.18%; Characterized by ¹H-NMR

### Step-7: Synthesis of Tert-butyl 7-hydroxyheptanoate (Int-12)

To a stirred solution of Int-11 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 70% EtOAc in Hexane (KMnO4 active)

Work up: Reaction mixture was cooled to 0° C., quenched with MeOH. Reaction mixture was directly concentrated under reduced pressure at 45° C. Crude was purified through combiflash at 15-20% Ethylacetae-Hexane to afford Int-12 as oily liquid.

Yield: 11 g, 58.8%; Characterized by ¹H-NMR

### Step-8: Synthesis of 7-(Tert-butoxy)-7-oxoheptyl 2-nonylundecanoate (Int-13)

To a stirred solution of 2-nonylundecanoic acid (Int-9) (8.0 g, 0.0255 mol) in DCM (160 mL, 20 Vol.) was added Int-12 (5.1 g, 0.0255 mol) followed by addition of EDC·HCl (9.7 g, 0.051 mol) and DMAP (1.5 g, 0.0127 mol) by maintaining temperature 5-10° C. DIPEA (17.7 mL, 0.102 mol) was added in a drop wise manner at same temperature. After complete addition, the reaction mixture was stirred at 25-30° C. for 48 h. Progress of the reaction was monitored by TLC. TLC shows new non-polar spot formation.

TLC eluent: 10% EtOAc in Hexane (KMnO₄ active)

Work up: Reaction mixture was quenched with 10% NaHCO₃ (200 mL×2) and extracted with DCM (200 mL×2). Organic layer was dried over anhydrous sodium sulphate and concentrated under reduced pressure at 45° C. Crude was purified through combiflash at 10-20% ethyl acetate-hexane to afford Int-13 as pale-yellow liquid.

Yield: 6.0 g, 50%; Characterized by ¹H-NMR

### Step-9: Synthesis of 7-[(2-nonylundecanoyl)oxy]heptanoic Acid (Int-5)

A stirred solution of Int-13 (6 g, 0.012 mol) in DCM (12 mL, 2 V) was cooled to at 0-10° C., Trifluoroaceticacid (48 mL, 8 V) was added in a dropwise manner at 0-5° C. Reaction mixture was stirred at 25-30° C. for 18 h. Progress of reaction monitored by TLC. TLC shows new polar spot formation.

TLC eluent: 10% MeOH in DCM

Work up: Reaction mixture was evaporated to dryness under reduced pressure at 45° C. Residue was dissolved in DCM (1×100 ml), neutralized with 10% sodium bicarbonate solution (100 ml×2). Organic layer was dried over anhydrous sodium sulphate and concentrated under reduced pressure at 45° C. to afford Int-5 as pale brown liquid.

Yield: 3.5 g, Crude; Characterized by ¹H-NMR

### Example 2: Synthesis of Lipid-2

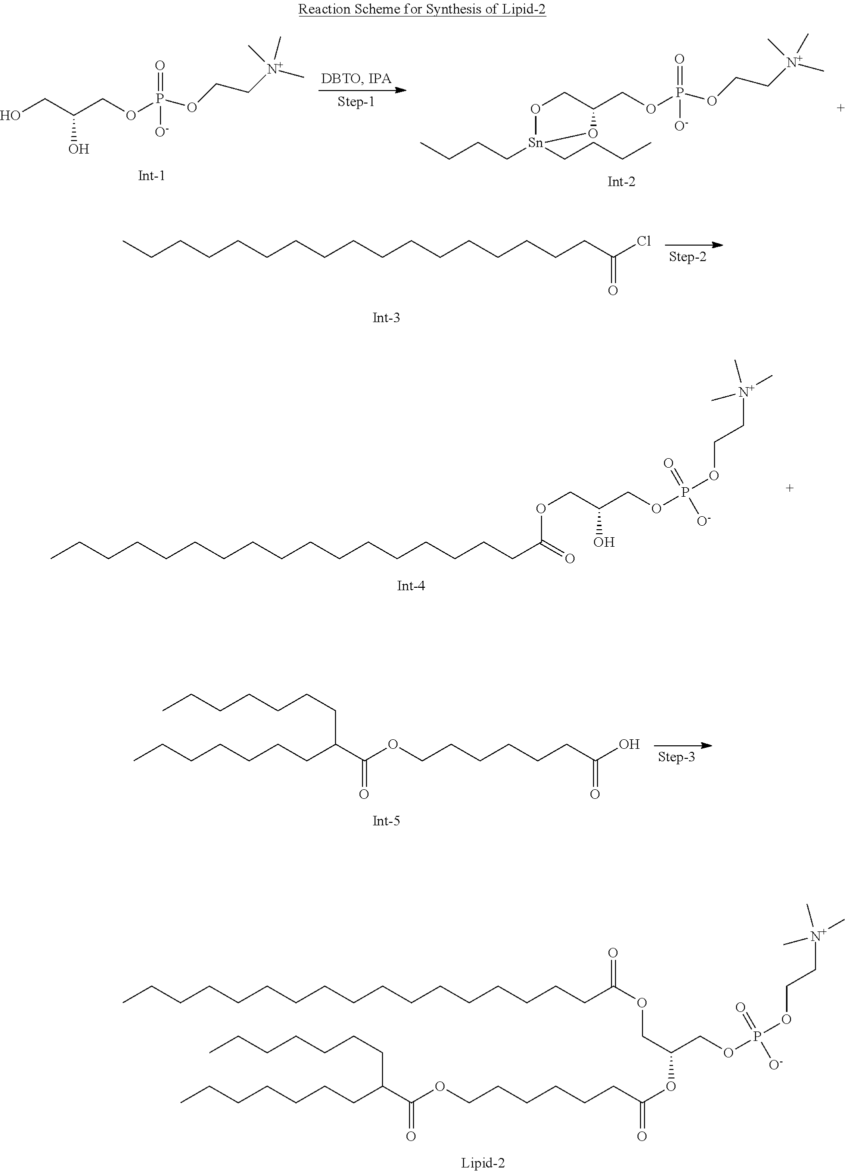

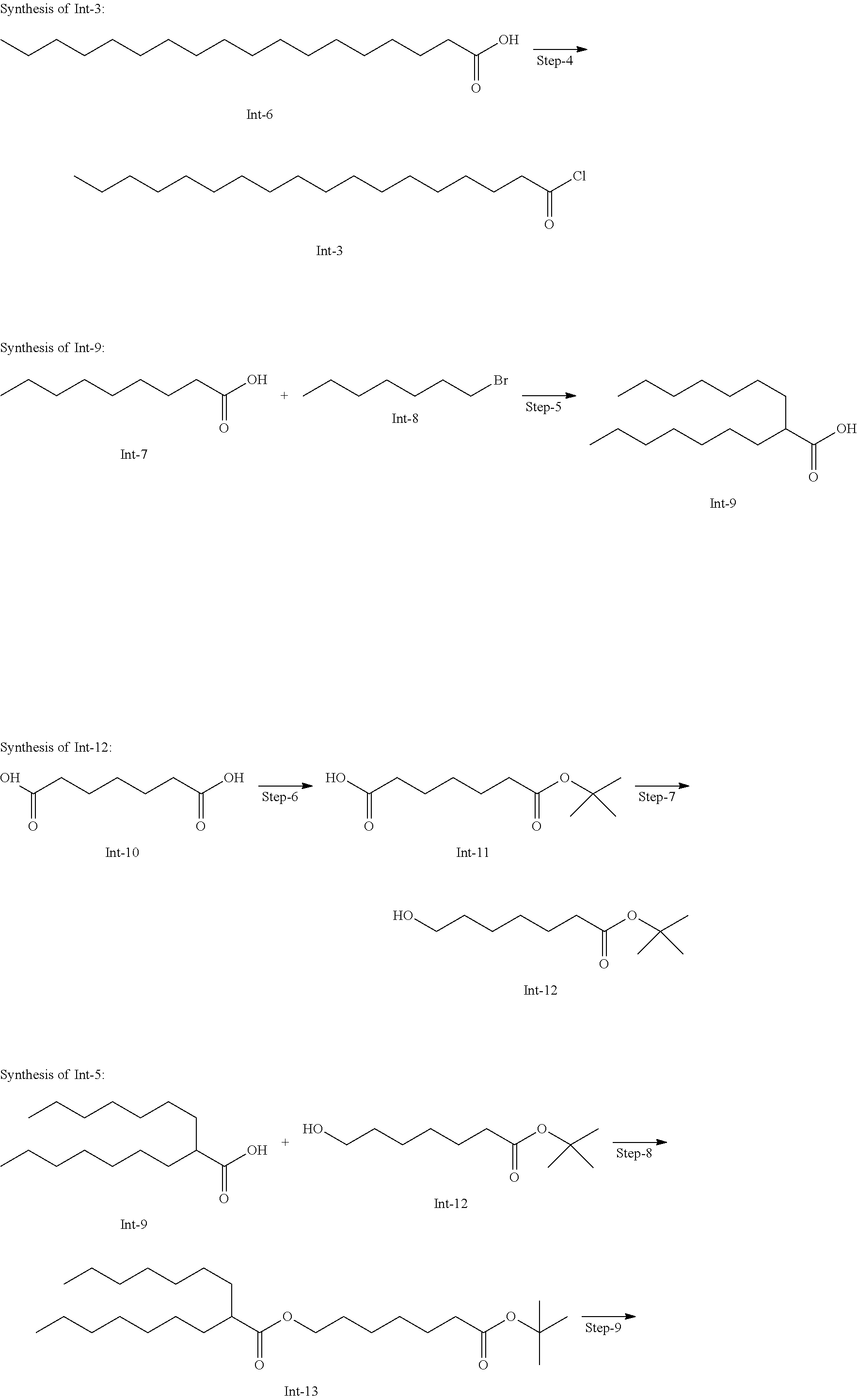

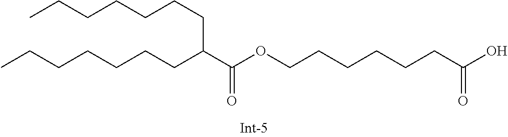 [SMILES: CCCCCCCC(CCCCCCC)C(=O)OCCCCCCC(=O)O]

### Synthetic Procedure

### Step-1&2: Synthesis of (2-{[(2R)-2-hydroxy-3-(octadecanoyloxy)propyl phosphonato]oxy}ethyl)trimethylazanium (Int-4)

Choline glycerophosphate (5 g, 0.0194 mol) and dibutyltin oxide (DBTO) (5.3 g, 0.0213 mol) were suspended in 2-propanol (250 mL) and refluxed for 18 h. The mixture was cooled to 0-5° C., TEA (3.3 mL, 0.0233 mol) and Stearoyl chloride (Int-3) (7.06 g, 0.0233 mol) were added. Resulting reaction mixture was stirred over a period of 18 h at 25-30° C. under argon atmosphere. Progress of the reaction was monitored by TLC. TLC shows new polar spot formation.

TLC eluent: (CH₂Cl₂/MeOH/H₂O: 65/25/4)-KMnO₄ active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Crude residue was dissolved in ethanol (100 mL) and precipitated with acetone (400 mL) at −10° C. to afford (2-{[(2R)-2-hydroxy-3-(octadecanoyloxy)propylphosphonato]oxy}ethyl)trimethylazanium (Int-4) as a white solid.

Yield: 6 g, 59.4%; Characterized by ¹H-NMR

### Step-3: Synthesis of (2-{[(2R)-2-({7-[(2-heptylnonanoyl)oxy]heptanoyl}oxy)-3-(octadecanoyloxy)propyl phosphonato]oxy}ethyl)trimethylazanium (Lipid-2)

To a solution of Int-4 (1.5 g, 0.0028 mol) in DCM (75 mL, 50 V) at 0 to 5° C., were added Int-5 (2.2 g, 0.0057 mol), 2-methyl-6-nitrobenzoic anhydride [MNBA](4.9 g, 0.0143 mol) and DMAP (3.4 g, 0.028 mol). Resulting reaction mixture was stirred for 48 h at 25-30° C. Progress of the reaction was monitored by TLC. TLC shows new non-polar spot formation.

TLC eluent: (IPA/EtOAc/H₂O: 8/5/4) Molybdenum blue reagent

Work up: Reaction mixture was directly concentrated under reduced pressure at 40° C. Residue was dissolved in DCM and DOWEX H⁺ (20% w/w) resin was added, stirred gently for 1 h at 25-30° C. After 1 h, TLC shows absence of DMAP and then DCM layer was filtered and concentrated under reduced pressure at 40° C. Crude was purified manually using Neutral alumina column chromatography using CH₂Cl₂:MeOH:H₂O (65/25/4) to afford Lipid-2 as brown semi-solid.

Yield: 600 mg, 24%; Characterized by ¹H-NMR, ³¹P-NMR and LCMS

LCMS: (EI, m/z) calcd for C₄₉H₉₆NO₁₀P [M+H]: 890.9; HPLC (CAD) Purity: 90.25%

¹H NMR (400 MHz, CDCl₃): 5.27-5.22 (1H, m), 4.46-4.42 (1H, dd, J=12 Hz), 4.27 (2H, m), 4.17-4.14 (1H, m), 4.08 (2H, t, J=6.4 Hz), 4.0 (2H, t, J=5.6 Hz), 3.65-3.63 (2H, m), 3.22 (9H, s), 2.37-2.30 (5H, m), 1.66-1.58 (8H, m), 1.47-1.44 (6H, m), 1.44-1.29 (48H, m), 0.9 (9H, t, J=7.2 Hz);

### Step-4: Synthesis of Octadecanoyl Chloride (Int-3)

A stirred solution of Stearic acid Int-6 (15 g, 0.052 mol) in DCM (150 mL, 10 V) was cooled to 0-10° C., Oxalyl chloride (9 mL, 0.1054 mol) was added in a drop wise manner at 0-5° C. and catalytic DMF (0.1 mL) was added. Resulting reaction was stirred for 5 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 40° C. to dryness to afford Int-3 as yellow liquid. Crude was taken as such for next step immediately.

Yield: 12 g (crude)

### Step-5: Synthesis of 2-Heptylnonanoic Acid (Int-9)

A solution of Int-7 (10 g, 0.0631 mol) in THF (300 mL, 30 V) was cooled to −5 to 0° C., NaH (60% in oil) (2.5 g, 0.0631 mol) was added in a dropwise manner by maintaining same temperature, stirred for 30 min. To the above reaction mixture LDA-2M in THF (63.19 mL, 0.1263 mol) was added in a drop wise manner by maintaining temperature −5 to 0° C., stirred for 30 min. 1-Iodononane (Int-8) (12.4 g, 0.0695 mol) was added at 0-10° C. The resultant reaction mixture was warmed to room temperature and refluxed at 45° C., 18 h. TLC monitored progress of the reaction. TLC shows non-polar spot formation along with SM.

TLC eluent: 20% in EtOAc in Hexane

Work up:—Reaction mass was cooled to −5-0° C., quenched with the addition of 1.5 N HCl by maintaining −5 to 0° C. The resultant reaction mixture was warmed to room temperature and extracted with ethyl acetate (1000 mL) and washed with purified water (800 mL).

Organic layer was dried over anhydrous sodium sulphate and concentrated under reduced pressure at 45° C. to afford yellow liquid. crude was purified through combiflash at 3-4% ethyl acetate-hexane to afford Int-9 as pale-yellow oil.

Yield: 10.0 g, 61.72%; Characterized by ¹H-NMR

### Step-6: Synthesis of 7-(Tert-butoxy)-7-oxoheptanoic Acid (Int-11)

To a stirred solution of Pimelic acid (Int-10) (20 g, 0.1248 mol) in THF (200 mL, 10 V) and tert-butanol (100 mL, 5 V) at 0° C., Boc anhydride (28.6 mL, 0.1248 mol) was added dropwise followed by DMAP (7.6 g, 0.0624 mol) in a dropwise manner and slowly brought to ambient temperature. Resulting reaction was stirred for 18 h at 25-30° C. Progress of the reaction was monitored by TLC. TLC shows new non-polar spot formation.

TLC eluent: 10% MeOH in DCM (KMnO4 active)

Work up: Reaction mixture was directly concentrated under reduced pressure at 45° C. Crude was purified through combiflash at 7-8% MeOH/DCM to afford Int-11 as oily liquid.

Yield: 9.5 g, 35.18%; Characterized by ¹H-NMR

### Step-7: Synthesis of Tert-butyl 7-hydroxyheptanoate (Int-12)

To a stirred solution of Int-11 (20 g, 0.092 mol) in THF (200 mL, 10 V) at 0° C., BH₃·DMS (17.5 mL, 0.1850 mol) was added dropwise and slowly brought to ambient temperature. Resulting reaction was stirred for 18 h at room temperature. Progress of the reaction was monitored by TLC. TLC shows new spot formation.

TLC eluent: 70% EtOAc in Hexane (KMnO4 active)

Work up: Reaction mixture was cooled to 0° C., quenched with MeOH. Reaction mixture was directly concentrated under reduced pressure at 45° C. Crude was purified through combiflash at 15-20% Ethylacetae-Hexane to afford Int-12 as oily liquid.

Yield: 11 g, 58.8%; Characterized by ¹H-NMR

### Step-8: Synthesis of 7-(tert-butoxy)-7-oxoheptyl 2-heptylnonanoate (Int-13)

To a stirred solution of Int-9 (10.0 g, 0.0390 mol) in DCM (200 mL, 20 Vol.) was added Int-12 (7.8 g, 0.0390 mol) followed by addition of EDC·HCl (14.9 g, 0.0780 mol) and DMAP (4.7 g, 0.0390 mol) by maintaining temperature 5-10° C. DIPEA (27.17 mL, 0.156 mol) was added in a drop wise manner at same temperature. After complete addition, the reaction mixture was stirred at 25-30° C. for 48 h. Progress of the reaction was monitored by TLC. TLC shows new non-polar spot formation.

TLC eluent: 10% EtOAc in Hexane (KMnO₄ active)

Work up: Reaction mixture was quenched with 10% NaHCO₃ (200 mL×2) and extracted with DCM (200 mL×2). Organic layer was dried over anhydrous sodium sulphate and concentrated under reduced pressure at 45° C. Crude was purified through combiflash at 4-5% ethyl acetate-hexane to afford Int-13 as colourless liquid.

Yield: 17.18 g, 52.38%; Characterized by ¹H-NMR

### Step-9: Synthesis of 7-[(2-Heptylnonanoyl)oxy]heptanoic Acid (Int-5)

A stirred solution of Int-13 (9 g, 0.0204 mol) in DCM (18 mL, 2 V) was cooled to at 0-10° C., Trifluoroacetic acid (72 mL, 8 V) was added in a dropwise manner at 0-5° C. Reaction mixture was stirred at 25-30° C. for 18 h. Progress of reaction monitored by TLC. TLC shows new polar spot formation.

TLC eluent: 10% MeOH in DCM

Work up: Reaction mixture was evaporated to dryness under reduced pressure at 45° C. Residue was dissolved in DCM (1×100 ml), neutralized with 10% sodium bicarbonate solution (100 ml×2). Organic layer was dried over anhydrous sodium sulphate and concentrated under reduced pressure at 45° C. to afford Int-5 as pale brown liquid. Yield: 7.8 g, Crude; Characterized by ¹H-NMR

### Example 3: Synthesis of Lipid-3

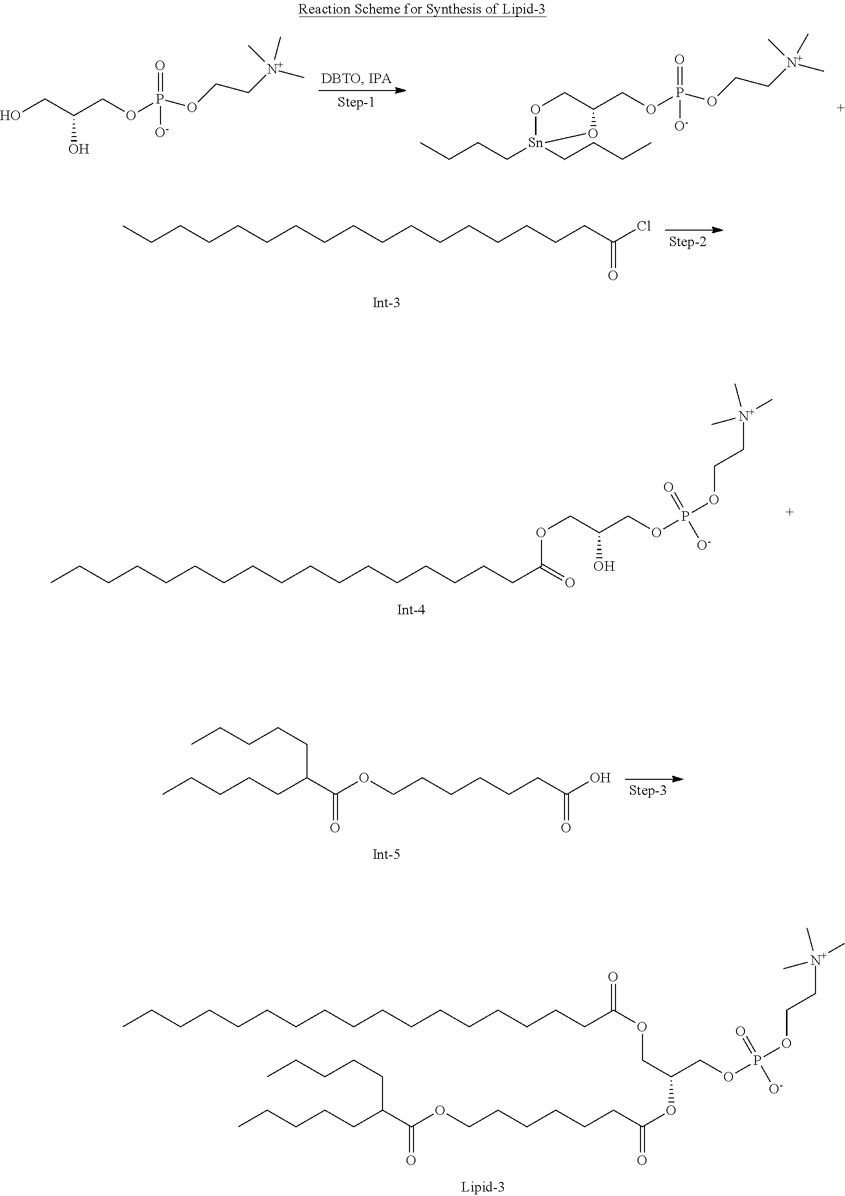

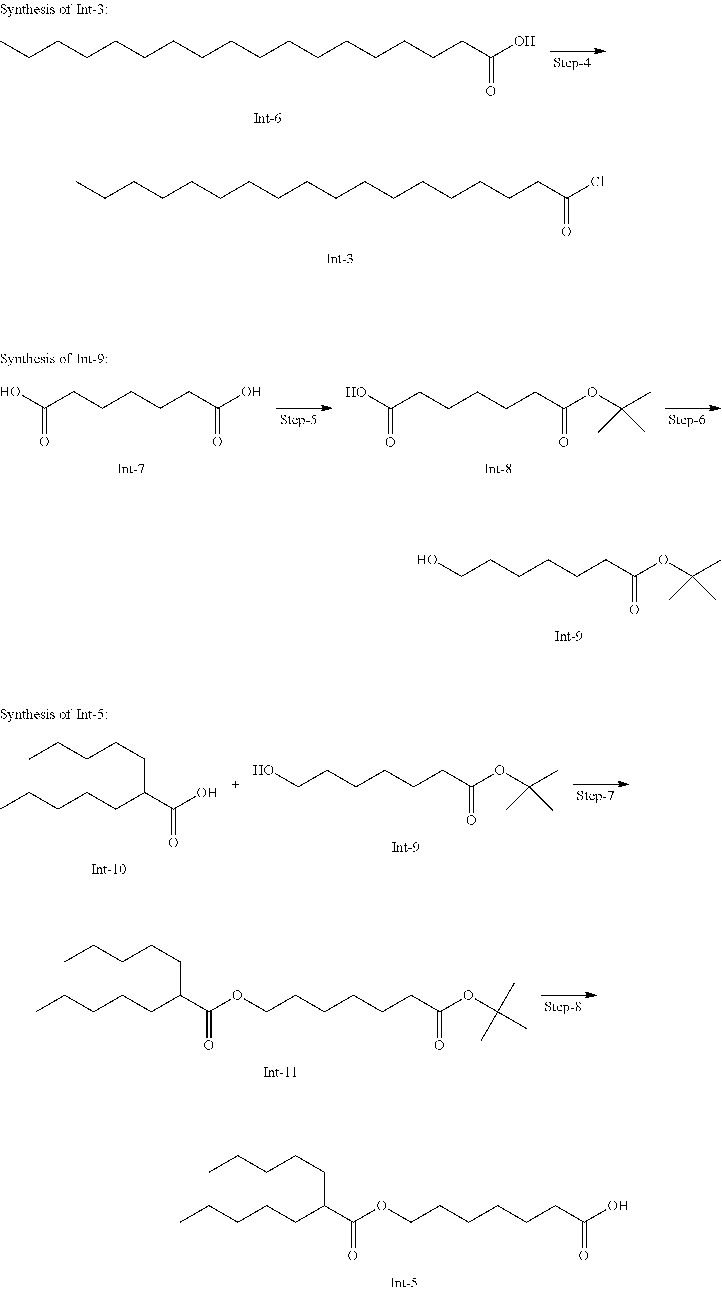

### Synthetic Procedure

### Step-1&2: Synthesis of (2-{[(2R)-2-hydroxy-3-(octadecanoyloxy)propyl phosphonato]oxy}ethyl)trimethylazanium (Int-4)

Choline glycerophosphate (5 g, 0.0194 mol) and dibutyltin oxide (DBTO) (5.3 g, 0.0213 mol) were suspended in 2-propanol (250 mL) and refluxed for 18 h. The mixture was cooled to 0-5° C., TEA (3.3 mL, 0.0233 mol) and Stearoyl chloride (Int-3) (7.06 g, 0.0233 mol) were added. Resulting reaction mixture was stirred over a period of 18 h at 25-30° C. under argon atmosphere. Progress of the reaction was monitored by TLC. TLC shows new polar spot formation.

TLC eluent: (CH₂Cl₂/MeOH/H₂O: 65/25/4)-KMnO₄ active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Crude residue was dissolved in ethanol (100 mL) and precipitated with acetone (400 mL) at −10° C. to afford (2-{[(2R)-2-hydroxy-3-(octadecanoyloxy)propylphosphonato]oxy}ethyl)trimethylazanium (Int-4) as a white solid.

Yield: 6 g, 59.4%; Characterized by ¹H-NMR

### Step-3: Synthesis of Trimethyl(2-{[(2R)-3-(octadecanoyloxy)-2-({7-[(2-pentylheptanoyl) oxy]heptanoyl}oxy)propyl phosphonato]oxy}ethyl)azanium (Lipid-3)

To a solution of Int-4 (1.5 g, 0.0028 mol) in DCM (60 mL, 40 V) at 0 to 5° C., were added Int-5 (1.13 g, 0.0034 mol), 2-methyl-6-nitrobenzoic anhydride [MNBA](4.9 g, 0.0143 mol) and DMAP (3.4 g, 0.0286 mol). Resulting reaction mixture was stirred for 48 h at 25-30° C. Progress of the reaction was monitored by TLC. TLC shows new nonpolar spot formation.

TLC eluent: TLC eluent: (IPA/EtOAc/H₂O: 8/5/4) Molybdenum blue reagent

Work up: Reaction mixture was directly concentrated under reduced pressure at 40° C. Residue was dissolved in DCM and DOWEX H⁺ (20% w/w) resin was added, stirred gently for 1 h at 25-30° C. After 1 h, TLC shows absence of DMAP and then DCM layer was filtered and concentrated under reduced pressure at 40° C. Crude was purified manually using Neutral alumina column chromatography using CH₂Cl₂:MeOH:H₂O (65/25/4) to afford Lipid-3 as brown semi-solid.

Yield: 600 mg, 25.1%; Characterized by ¹H-NMR, ³¹P-NMR and LCMS

LCMS: (EI, m/z) calcd for C₄₅H₈₈NO₁₀P [M+H]: 834.9; HPLC (CAD) Purity: 82.12%

¹H NMR (400 MHz, MeOD): 5.27-5.22 (1H, m), 4.46-4.42 (1H, dd, J=12 Hz), 4.27-4.25 (2H, m), 4.19-4.14 (1H, m), 4.08 (2H, t, J=6.4 Hz), 4.0 (2H, t, J=5.6 Hz), 3.65-3.63 (2H, m), 3.22 (9H, s), 2.37-2.30 (5H, m), 1.66-1.58 (8H, m), 1.47-1.44 (6H, m), 1.44-1.29 (40H, m), 0.9 (9H, t, J=7.2 Hz);

### Step-4: Synthesis of Octadecanoyl Chloride (Int-3)

A stirred solution of Stearic acid Int-6 (15 g, 0.052 mol) in DCM (150 mL, 10 V) was cooled to 0-10° C., Oxalyl chloride (9 mL, 0.1054 mol) was added in a drop wise manner at 0-5° C. and catalytic DMF (0.1 mL) was added. Resulting reaction was stirred for 5 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 40° C. to dryness to afford Int-3 as yellow liquid. Crude was taken as such for next step immediately.

Yield: 12 g (crude);

### Step-5: Synthesis of 7-(Tert-butoxy)-7-oxoheptanoic Acid (Int-8)

To a stirred solution of Pimelic acid (Int-7) (20 g, 0.1248 mol) in THF (200 mL, 10 V) and tert-butanol (100 mL, 5 V) at 0° C., Boc anhydride (28.6 mL, 0.1248 mol) was added dropwise followed by DMAP (7.6 g, 0.0624 mol) in a dropwise manner and slowly brought to ambient temperature. Resulting reaction was stirred for 18 h at room temperature.

Progress of the reaction was monitored by TLC. TLC shows new non-polar spot formation.

TLC eluent: 10% MeOH in DCM (KMnO₄ active)

Work up: Reaction mixture was directly concentrated under reduced pressure at 45° C. Crude was purified through combiflash at 7-8% MeOH/DCM to afford Int-8 as oily liquid.

Yield: 9.5 g, 35.18%; Characterized by ¹H-NMR

### Step-6: Synthesis of Tert-butyl 7-hydroxyheptanoate (Int-9)

To a stirred solution of Int-8 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 70% EtOAc in Hexane (KMnO4 active)

Work up: Reaction mixture was cooled to 0° C., quenched with MeOH. Reaction mixture was directly concentrated under reduced pressure at 45° C. Crude was purified through combiflash at 15-20% ethyl acetate-Hexane to afford Int-9 as oily liquid.

Yield: 11 g, 58.8%; Characterized by ¹H-NMR

### Step-7: Synthesis of 7-(Tert-butoxy)-7-oxoheptyl 2-pentylheptanoate (Int-11)

To a stirred solution of pentylheptanoic Acid (Int-10) (5.0 g, 0.249 mol) in DCM (100 mL, 20 Vol.) was added Int-9 (5.5 g, 0.074 mol) followed by addition of EDC·HCl (9.5 g, 0.0499 mol) and DMAP (1.5 g, 0.0124 mol) by maintaining temperature 5-10° C. DIPEA (17.8 mL, 0.0996 mol) was added in a drop wise manner at same temperature. After complete addition, the reaction mixture was stirred at 25-30° C. for 48 h. Progress of the reaction was monitored by TLC. TLC shows new non-polar spot formation.

TLC eluent: 10% EtOAc in Hexane (KMnO4 active)

Work up: Reaction mixture was quenched with 10% NaHCO₃ (200 mL×2) and extracted with DCM (200 mL×2). Organic layer was dried over anhydrous sodium sulphate and concentrated under reduced pressure at 45° C. Crude was purified through combiflash at 5-10% ethyl acetate-hexane to afford Int-11 as colourless liquid.

Yield: 6.0 g, 63.15%, Characterized by ¹H-NMR

### Step-8: Synthesis of 7-[(2-Pentylheptanoyl)oxy]heptanoic Acid (Int-5)

A stirred solution of Int-11 (6 g, 0.0156 mol) in DCM (12 mL, 2 V) was cooled to at 0-10° C., Trifluoroacetic acid (48 mL, 8 V) was added in a dropwise manner at 0-5° C. Reaction mixture was stirred at 25-30° C. for 18 h. Progress of reaction monitored by TLC. TLC shows new polar spot formation.

TLC eluent: 10% MeOH in DCM

Work up: Reaction mixture was evaporated to dryness under reduced pressure at 45° C. Residue was dissolved in DCM (1×100 ml), neutralized with 10% sodium bicarbonate solution (100 ml×2). Organic layer was dried over anhydrous sodium sulphate and concentrated under reduced pressure at 45° C. to afford Int-5 as pale brown liquid. Yield: 4.0 g, Crude; Characterized by ¹H-NMR

### Example 4: Synthesis of Lipid-4

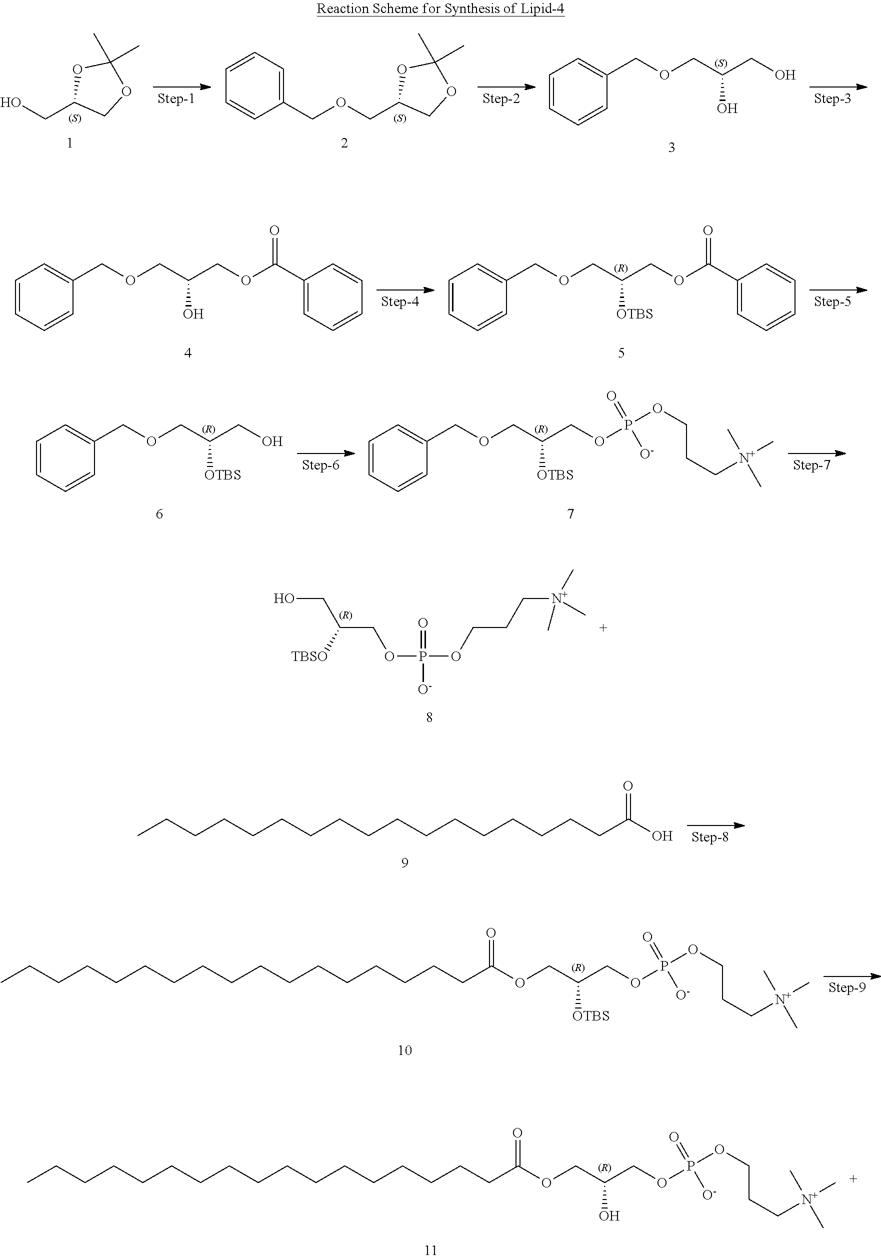

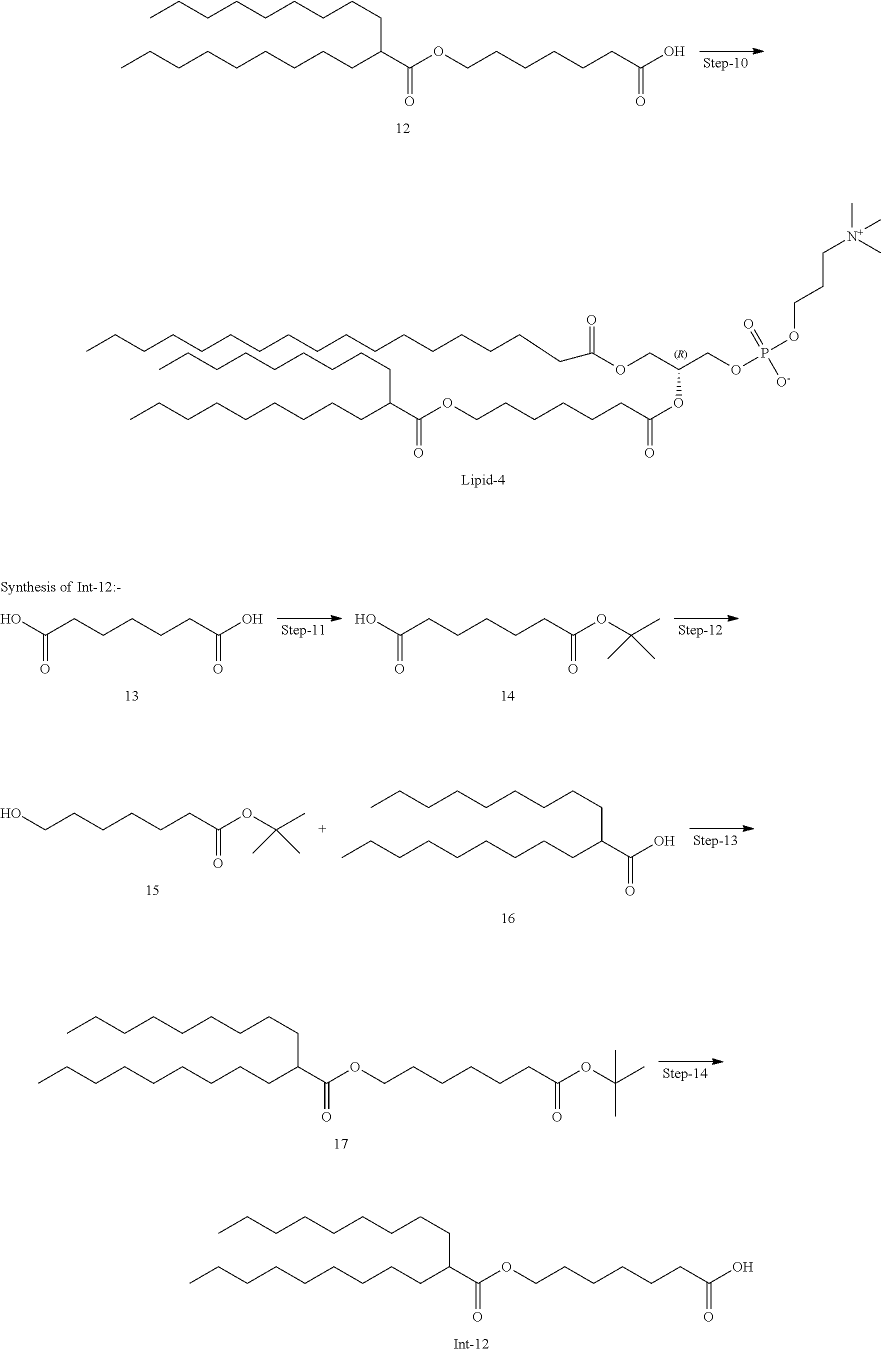

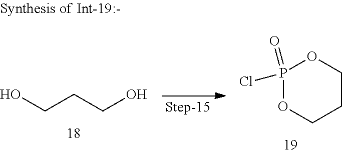 [SMILES: OCCCO ; O=P1(Cl)OCCCO1]

### Synthetic Procedure

### Step-1:—Synthesis of 4-Benzyloxymethyl-2,2-dimethyl-[1,3]dioxolane (Int-2)

In a 3 neck RB (2,2-Dimethyl-[1,3]dioxolan-4-yl)-methanol (20 g, 0.0151 mol) was taken in THF (500 mL, 25V) and DMF (500 mL, 25V) and cooled for 0° C. Added NaH (60% in oil) (6.65 g, 0.1664 mol) dropwise (1.66 g×4 lots) over interval of 5 minutes, followed by Benzyl chloride (19.1 mL, 0.1664 mol). RM was brought to ambient temperature and stirred over a period of 18 h at 25-30° C. under argon atmosphere. Progress of the reaction was monitored by TLC. TLC shows new nonpolar spot formation.

TLC eluent: 40% Ethyl acetate in hexane-KMnO₄ active

Work up: Reaction mass was cooled to 0° C. diluted with methanol (1000 mL) and stirred for 30 minutes at same temperature. RM was concentrated under reduced pressure.

Obtained residue was diluted with water (800 mL) and extracted with DCM (750 mL×2).

Combined organic layers were dried over anhydrous sodium sulphate and concentrated under reduced pressure at 45° C. to afford crude. Crude was purified through combiflash at 8-10% ethyl acetate-hexane to afford Int-2 as pale-yellow oily liquid.

Yield: 30.01 g (89.2%); Characterized by ¹H NMR

### Step-2: Synthesis of 3-Benzyloxy-propane-1,2-diol (Int-3)

To a solution of Int-2 (10 g, 0.045 mol) in methanol (50 mL, 5 V) and water (50 mL, 5 V) at 0-5° C., added DOWEX H⁺ resin (4 g, 40% w/w). Resulting reaction mixture was stirred for 18 h at 25-30° C. Progress of the reaction was monitored by TLC. TLC shows new polar spot formation.

TLC eluent: 50% Ethyl acetate in hexane-KMnO₄ active

Work up: Reaction mixture was filtered and obtained filtrate was concentrated under reduced pressure at 40° C. Residue was diluted with water (300 mL) and extracted with DCM (450 mL×2). Combined organic layers were dried over anhydrous sodium sulphate and concentrated under reduced pressure at 45° C. to afford Int-3.

Yield: 8.2 g (crude); Characterized by ¹H NMR

### Step-3: Synthesis of Benzoic acid 3-benzyloxy-2-hydroxy-propyl ester (Int-4)

To a stirred solution of Int-3 (8 g, 0.0439 mol) in ACN (200 mL, 25 V) added 2 diphenyl boronyloxyethanamine (0.988 g, 0.00439 mol), Diisopropylethylamine (11.46 mL, 0.0658 mol) followed by Benzoyl chloride (7.64 mL, 0.0658 mol) dropwise at ambient temperature. Resulting reaction mixture was stirred for 2 h at room temperature. Progress of the reaction was monitored by TLC. TLC shows new non-polar spot formation.

TLC eluent: 50% Ethyl acetate in hexane-KMnO₄ active

Work up: Reaction mixture was quenched with water (400 mL) and extracted with Ethyl acetate (250 mL×3), combined organic layers were dried over anhydrous sodium sulphate and concentrated under reduced pressure at 45° C. to afford Int-4.

Yield: 16.7 g (Crude); Characterized by ¹H NMR

### Step-4: Synthesis of Benzoic acid 3-benzyloxy-2-(tert-butyl-dimethyl-silanyloxy)-propyl ester (Int-5)

To a stirred solution of Int-4 (16.5 g, 0.0576 mol) in DMF (247.5 mL, 15 V) at 0° C. added Imidazole (11.76 g, 0.0864 mol) followed by Tert-butyldimethylsilylchloride in a dropwise manner. Reaction mixture was brought to ambient temperature and stirred for 18 h. Progress of the reaction was monitored by TLC. TLC shows new nonpolar spot formation.

TLC eluent: 10% EtOAc in Hexane (KMnO4 active)

Work up: Reaction mixture was quenched with ice-cold water (500 mL) and extracted with Ethyl acetate (300 mL×3), combined organic layers were dried over anhydrous sodium sulphate and concentrated under reduced pressure at 45° C. to afford crude. Crude was purified through combiflash at 5-6% EtOAC/Hexane to afford Int-5 as pale-yellow oily liquid.

Yield: 13.31 g (57.65%); Characterized by ¹H NMR

### Step-5: Synthesis of 3-Benzyloxy-2-(tert-butyl-dimethyl-silanyloxy)-propan-1-ol (Int-6)

To a stirred solution of Int-5 (12 g, 0.0299 mol) in THF (150 mL, 5 mL/mol) at room temperature added Ethyl magnesium bromide (105 mL, 0.104 mol) in a dropwise manner. Reaction mixture stirred for 2 h at ambient temperature. Progress of the reaction was monitored by TLC. TLC shows new polar spot formation.

TLC eluent: 40% in Ethyl acetate in Hexane-KMnO₄ active

Work up:—Reaction mass was cooled to −5 to 0° C., quenched with the addition of cold saturated NH₄Cl solution (1000 mL) by maintaining −5 to 0° C. The resultant reaction mixture was warmed to room temperature and extracted with ethyl acetate (500 mL×3). Organic layer was dried over anhydrous sodium sulphate and concentrated under reduced pressure at 45° C. to afford crude. Crude was purified through combiflash at 8-9% ethyl acetate-hexane to afford Int-6 as pale-yellow oily liquid.

Yield: 8 g (90%); Characterized by ¹H NMR

### Step-6: Synthesis of Int-7

To a stirred solution of Int-6 (2 g, 0.00674 mol) in DCM (16 mL, 8 V) at 0° C. added DMAP (82.4 mg, 0.000674 mol) followed by Triethylamine (9.44 mL, 0.0674 mol) in a dropwise manner, added Int-19 (5.27 g, 0.0337 mol) dissolved in DCM (16 mL, 8 V) in a dropwise manner, after completion of addition reaction mixture was stirred at 0° C. for 60 minutes and later slowly brought to ambient temperature and stirred for 20 h. Progress of the reaction was monitored by TLC. TLC shows new spot formation Reaction mixture was concentrated under reduced pressure at 40° C. and obtained residue was diluted with THF (40 mL) and filtered through celite bed, obtained filtrate was concentrated under reduced pressure at 40° C. and dried over high vacuum for 60 minutes, obtained crude was diluted with ACN (20 mL, 15V) and transferred to autoclave vessel and cooled to −60 C°. In another RB Trimethylamine gas (9.96 g, 25 eq.) was purged to 30 mL of ACN at −60° C. and added to previously cooled autoclave vessel containing crude at −60° C. Reaction mixture slowly brought to ambient temperature and heated to 65° C. for 18 h.

Progress of the reaction was monitored by TLC. TLC shows new spot formation.

TLC eluent: 40% Methanol in DCM, 1 mL Aq NH₃ (KMnO4 and molybdenum blue active)

Work up: Reaction mixture was concentrated under reduced pressure at 40° C. Crude was purified by column manually at 10% Methanol in DCM, 1 mL aqueous NH₃ in silica to afford Int-7 as pale brown sticky solid.

Yield: 0.966 g (34.4%); Characterized by ¹H-NMR and LCMS

### Step-7: Synthesis of Int-8

A stirred solution of Int-7 (2.8 g, 0.00588 mol) in Methanol (112 mL, 40 V) was degassed for 10 minutes with argon, added 10% Pd/C (1.31 g, 47% w/w). Reaction mixture was heated to 35° C. with 1 kg/cm² pressure for 24 h. Progress of reaction monitored by TLC.

TLC shows new polar spot formation.

TLC eluent: 60% Methanol in DCM, 2 mL Aq NH₃ (KMnO4 active)

Work up: Reaction mixture was filtered through celite bed and filtrate was evaporated to dryness under reduced pressure at 45° C. Crude was purified by column manually at 7-10% Methanol in DCM, 1 mL water in neutral alumina to afford Int-8 as off-white sticky solid.

Yield: 0.97 g (42.9%); Characterized by ¹H-NMR and LCMS

### Step-8: Synthesis of Int-10

To a stirred solution of Int-8 (0.95 g, 0.00246 mol) in DMF (23.75 mL, 25 V) was added Int-9 (2.29 g, 0.00808 mol), DMAP (0.96 g, 0.00787 mol) and DIC (1.7 mL, 0.0108 mol). Reaction mixture was stirred at 35° C. for 24 h. Progress of reaction monitored by TLC.

TLC shows new nonpolar spot formation.

TLC eluent: 30% Methanol in DCM, 1 mL Aq NH₃ (KMnO4 and molybdenum blue active)

Work up: Reaction mixture was concentrated under reduced pressure at 40° C. Crude was purified by column manually at 15% Methanol in DCM, 1 mL Aq NH₃ in silica to afford Int-10 as off-white gummy liquid.

Yield: 0.9 g (56.2%); Characterized by ¹H-NMR and LCMS

### Step-9: Synthesis of Int-11

Int-10 (0.9 g, 0.00138 mol) was taken 2 neck RB, was added a solution of 0.1% TFA [(0.157 g, 0.00138 mol) in water (157 mL)]. Reaction mixture was stirred at 25-30° C. for 16 h. Progress of reaction monitored by TLC. TLC shows new polar spot formation.

TLC eluent: 30% Methanol in DCM, 1 mL Aq NH₃ (KMnO4 and molybdenum blue active)

Work up: Reaction mixture was lyophilized to get Int-11.

Yield: 0.81 g (Crude); Characterized by ¹H-NMR

### Step-10: Synthesis of Lipid-4

To a stirred solution of Int-11 (0.8 g, 0.00122 mol) in DCM (32 mL, 40 V) was added Int-12 (2.43 g, 0.00552 mol), DMAP (0.6 gg, 0.00491 mol) and DIC (1.57 mL, 0.00997 mol). Reaction mixture was stirred at 25-30° C. for 24 h. Progress of reaction monitored by TLC. TLC shows new nonpolar spot formation.

TLC eluent: 30% Methanol in DCM, 1 mL Aq NH₃ (KMnO4 and molybdenum blue active)

Work up: Reaction mixture was concentrated under reduced pressure at 40° C. Crude was purified by column manually at 15% Methanol in DCM, 1 mL Aq NH₃ in silica to afford Lipid-4 as off-white semisolid.

Yield: 0.54 g (45.8%); Characterized by ¹H-NMR and LCMS; HPLC Purity: 68.88%

LCMS: (EI, m/z) calcd for C₅₄H₁₀₆NO₁₀P [M+H]⁺: 960.9; HPLC (CAD) Purity: 68.88%

¹H NMR (400 MHz, CD₃OD): 5.26 (1H, s), 4.47-4.4 (1H, m), 4.18-4.14 (2H, dd, J=4.8 Hz,), 4.09-4.0 (2H, m), 3.98-3.93 (3H, m), 3.51-3.41 (2H, m), 3.34 (2H, s), 3.3-3.27 (4H, m), 3.15-3.07 (9H, m), 2.37-2.28 (4H, m), 2.11-2.01 (2H, m), 1.71-1.55 (8H, m), 1.47-1.37 (10H, m), 1.39-1.25 (47H, m), 0.966-0.85 (9H, m);

### Step-11: Synthesis of Heptanedioic acid mono-tert-butyl ester (Int-14)

To a stirred solution of Pimelic acid Int-13 (30 g, 0.187 mol) in THF (300 mL, 10 V) and tert-butanol (150 mL, 5 V) at 0° C., Boc anhydride (40.8 g, 0.187 mol) was added dropwise followed by DMAP (11.49 g, 0.0936 mol) in a dropwise manner and slowly brought to ambient temperature. Resulting reaction was stirred for 18 h at ambient temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 45° C. Crude was purified through combiflash at 30% EtOAc/Hexane to afford Int-14 as oily liquid.

Yield: 16.87 g (41.65%); Characterized by ¹H-NMR

### Step-12: Synthesis of 7-Hydroxy-heptanoic acid tert-butyl ester (Int-15)

To a stirred solution of Int-14 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at ambient temperature. Progress of the reaction was monitored by TLC. TLC shows new spot formation.

TLC eluent: 50% EtOAc in Hexane (KMnO4 active)

Work up: Reaction mixture was cooled to 0° C., quenched with MeOH (500 mL). Reaction mixture was directly concentrated under reduced pressure at 45° C. Crude was purified through combiflash at 24-25% EtOAc/Hexane to afford Int-15 as oily liquid.

Yield: 8.63 g (46.18%); Characterized by ¹H-NMR

### Step-13: Synthesis of 2-Nonyl-undecanoic acid 6-tert-butoxycarbonyl-hexyl ester (Int-17)

To a stirred solution of Int-15 (1.94 g, 0.0096 mol) in DCM (38.8 mL, 20 Vol.) was added Int-16 (3 g, 0.0096 mol) followed by addition of EDC·HCl (2.98 g, 0.0192 mol) and DMAP (0.58 g, 0.0048 mol) by maintaining temperature 5° C.-10° C. DIPEA (6.6 mL, 0.038 mol) was added in a drop wise manner at same temperature. After complete addition, the reaction mixture was stirred at 25-30° C. for 24 h. Progress of the reaction was monitored by TLC. TLC shows new nonpolar spot formation.

TLC eluent: 30% EtOAc in Hexane (KMnO4 active)

Work up: Reaction mixture was quenched with 10% NaHCO₃ (300 mL) and extracted with DCM (300 mL×3). Combined organic layers were dried over anhydrous sodium sulphate and concentrated under reduced pressure at 45° C. Crude was purified through combiflash at 1-3% EtOAc/Hexane to afford Int-17 as pale-yellow liquid.

Yield: 3.6 g (75.6%); Characterized by ¹H-NMR

### Step-14: Synthesis of 2-Nonyl-undecanoic acid 6-carboxy-hexyl ester (Int-12)

A stirred solution of Int-17 (3.5 g, 0.00704 mol) in DCM (7 mL, 2 V) was cooled to at 0-10° C., Trifluoroaceticacid (28 mL, 8 V) was added in a dropwise manner at 0-5° C. Reaction mixture was stirred at 25-30° C. for 18 h. Progress of reaction monitored by TLC. TLC shows new polar spot formation.

TLC eluent: 70% EtOAc in Hexane (KMnO4 active)

Work up: Reaction mixture was evaporated to dryness under reduced pressure at 45° C. Residue was dissolved in DCM (2×100 ml), neutralized with 10% sodium bicarbonate solution (300 mL). Organic layer was dried over anhydrous sodium sulphate and concentrated under reduced pressure at 45° C. Crude was purified by combiflash at 18-20% EtOAc/Hexane to afford Int-12 as pale brown liquid.

Yield: 3 g (96.7%); Characterized by ¹H-NMR

### Step-15: Synthesis of 2-Chloro-[1,3,2]dioxaphosphinane 2-oxide (Int-19)

A solution of propane-1,3-diol (15 g, 0.197 mol) and triethylamine (54.9 ml, 0.394 mol) in dichloromethane (90 ml) and a solution of phosphorus oxychloride (18.43 mL, 0.197 mol) in dichloromethane (105 ml) were added slowly and simultaneously with stirring to dichloromethane (105 ml) at 0° C. The reaction mixture was stirred at the same temperature for 20 min and a further 30 min at rt. The solvents were removed and the obtained solid was extracted with diethyl ether (300 mL) and filtered, and the filtrate was evaporated to dryness to get Int-19 as white crystalline solid.

Yield: 18 g (58.3%); Characterized by ¹H-NMR

### Example 5: Synthesis of Lipid-5

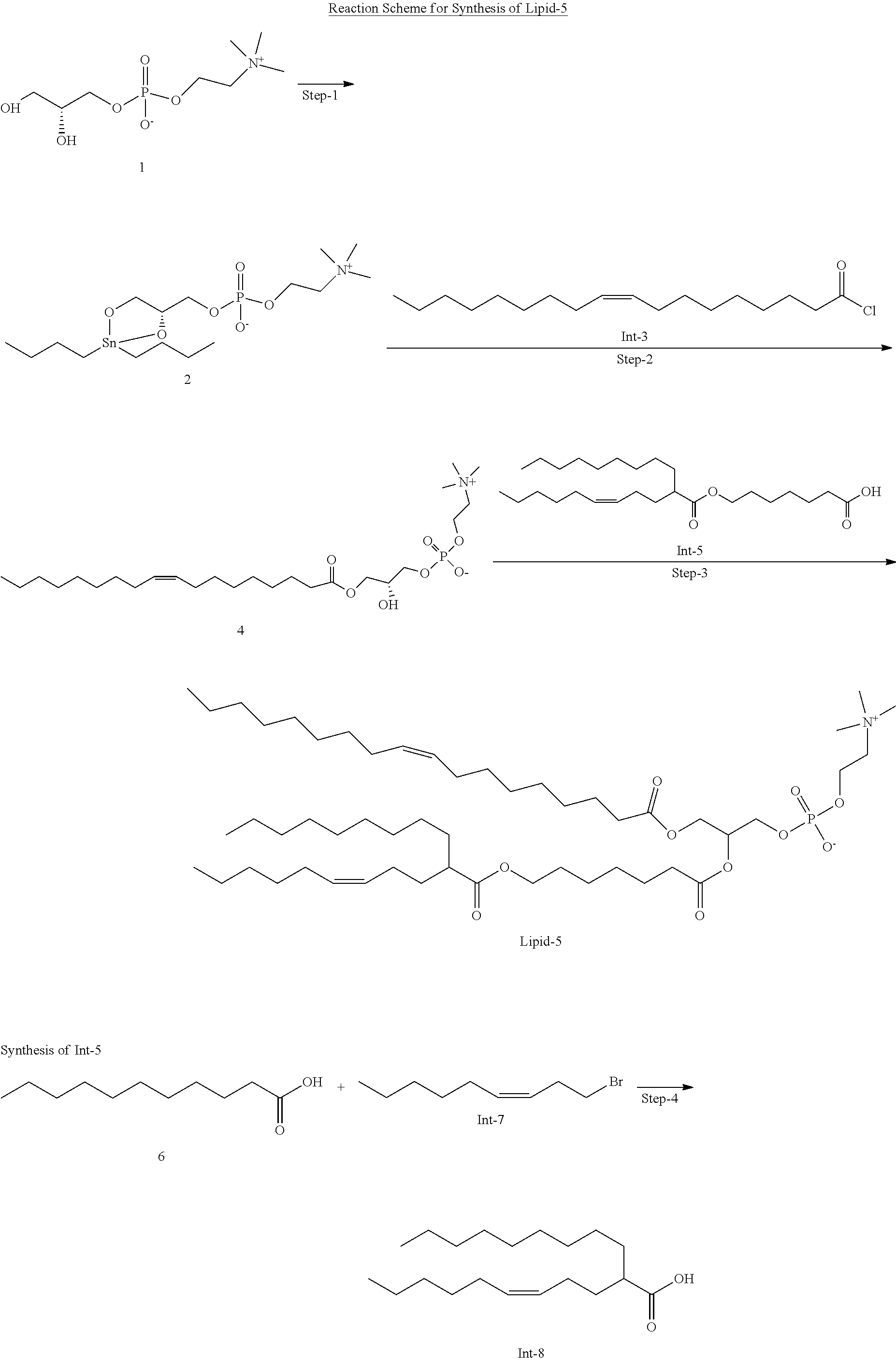

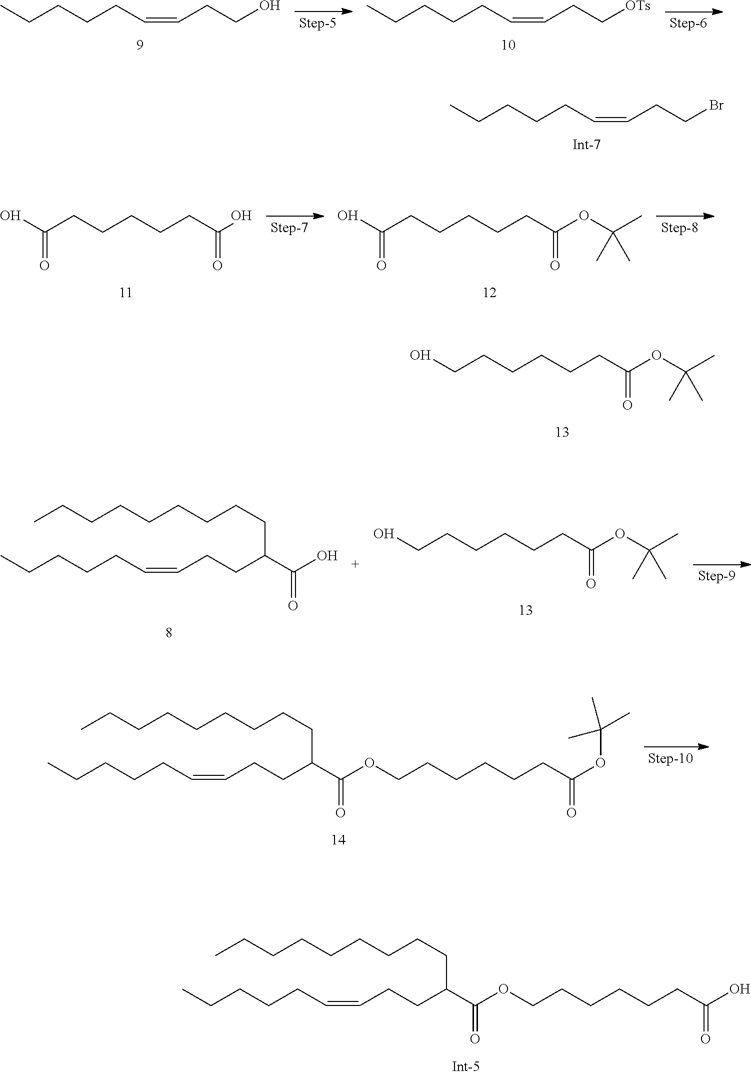

### Synthetic Procedure

### Step-1&2: Synthesis of Int-4

Choline glycerophosphate (2 g, 0.0077 mol) and dibutyltin oxide (DBTO) (2.12 g, 0.00854 mol) were suspended in 2-propanol (100 mL) and refluxed for 18 h at 90° C. The mixture was cooled to 0 to 5° C., TEA (1.3 mL, 0.00932 mol) and Int-3 (7.01 g, 0.0233 mol) were added. Resulting reaction mixture was stirred over a period of 36 h at 25-30° C. under argon atmosphere. Progress of the reaction was monitored by TLC. TLC shows new polar spot formation.

TLC eluent: (CH₂Cl₂/MeOH/Aq NH₃: 60/40/15)-KMnO₄ and Molybdenum blue active

Work up: Reaction mixture was diluted with purified water (250 mL) and extracted with heptane (250 mL). The water-alcohol solution was extracted three times with heptane (3×250 mL) and resulting aqueous layer evaporated under reduced pressure at 45° C. to dryness to get crude. Obtained crude was purified by column using 20% MeOH/DCM & 1% H₂O to get Int-4 as a white sticky solid.

Yield: 2.1 g (51.8%); Characterized by ¹H-NMR

### Step-3: Synthesis of Lipid-5

To a solution of Int-4 (1.3 g, 0.00249 mol) in DCM (65 mL, 50 V) were added Int-5 (0.95 g, 0.00448 mol), 2-methyl-6-nitrobenzoic anhydride (4.37 g, 0.0127 mol) and DMAP (3.04 g, 0.0249 mol) at 0-5° C. Resulting reaction mixture was stirred for 48 h at 25-30° C. Progress of the reaction was monitored by TLC. TLC shows new non polar spot formation.

TLC eluent: (20% MeOH/DCM & 0.2 mL Aq NH₃) Molybdenum blue & KMnO₄ active

Work up: Reaction mixture was directly concentrated under reduced pressure at 40° C. Residue was dissolved in DCM and was purified manually using neutral alumina column chromatography using 5% MeOH/DCM & 1% H₂O to obtained partially purified product, which was washed with water to remove DMAP and dried to afford Lipid-5 as pale green semi-solid.

Yield: 0.7 g (29.9%); Characterized by ¹H-NMR and Q-TOF QTOF: (EI, m/z) calcd for C₅₃H₁₀₀O₁₀P [M+H]⁺: 942.72; HPLC (CAD) Purity: 59.2%

¹H NMR (400 MHz, CD₃OD): 5.38-5.31 (4H, m), 5.26 (1H, m), 4.46-4.31 (1H, m), 4.27 (2H, m), 4.19-0.14 (1H, m), 4.1-4.07 (2H, m), 4.01-4 (2H, m), 3.64 (2H, m), 3.22 (9H, s), 2.37-2.29 (5H, m), 2.02-2.01 (8H, m), 1.63 (8H, m), 1.53-1.29 (48H, m), 0.913-0.88 (9H, m);

### Step-4: Synthesis of 2-Nonyl-undec-5-enoic Acid (Int-8)

In a 3 neck RB, THF (45 mL, 5 V) was cooled to −5 to 0° C. and was added LDA (2M in THF) (43.47 mL, 0.0869 mol) in a drop wise manner by maintaining temperature −5-0° C. After completion of addition, a solution of Int-6 (9 g, 0.0483 mol) in THF (45 mL, 5V) was added dropwise, followed by HMPA (9.49 mL, 0.0545 mol) dropwise. RM was immediately brought to ambient temperature and stirred for 30 minutes. RM was once again cooled to 0 to −5° C. and added Int-7 (10.89 g, 0.0531 mol) in a single addition. RM was immediately brought to ambient temperature and stirred for 4 h at RT. RM was monitored through TLC. TLC shows non-polar spot formation along with SM.

TLC eluent: 20% in EtOAc in Hexane

Work up:—Reaction mass was cooled to −5-0° C., quenched with the addition of cold 1.5 N HCl (300 mL) by maintaining −5 to 0° C. The resultant reaction mixture was warmed to room temperature and extracted with ethyl acetate (500 mL×2). Organic layer was dried over anhydrous sodium sulphate and concentrated under reduced pressure at 45° C. to afford crude. Crude was purified through combiflash at 2-3% ethyl acetate-hexane to afford Int-8 as pale-yellow oily liquid.

Yield: 5.81 g (38%); Characterized by ¹H-NMR

### Step-5: Synthesis of Toluene-4-sulfonic acid non-3-enyl ester (Int-10)

To a stirred solution of Int-9 (18 g, 0.126 mol) in DCM (180 mL, 10 V) at 0° C. added TEA (61.4 mL, 0.441 mol) dropwise followed by Tosyl chloride (48.04 g, 0.252 mol) in a dropwise manner and slowly brought to ambient temperature. Resulting reaction was stirred for 18 h at room temperature. Progress of the reaction was monitored by TLC. TLC shows new nonpolar spot formation along with unreacted Tosyl chloride.

TLC eluent: 20% EtOAc in hexane (KMnO₄ active)

Work up: Reaction mixture was quenched with cold water (500 mL) and extracted with EtOAc (300 mL×3). Combined organic layers dried over Na₂SO₄, concentrated under reduced pressure at 45° C. Crude was purified through combiflash at 2-3% EtOAc/Hexane to afford Int-10 as oily liquid.

Yield: 20 g, (Impure); Characterized by ¹H-NMR

### Step-6: Synthesis of 1-Bromo-non-3-ene (Int-7)

To a stirred solution of Int-10 (15 g, 0.0523 mol) in THF (150 mL, 10 V) at RT, added Magnesium bromide ethyl etherate (14.87 g, 0.0576 mol) dropwise, RM was heated to 40° C. for 16 h. Progress of the reaction was monitored by TLC. TLC shows new nonpolar spot formation.

TLC eluent: 10% EtOAc in hexane (KMnO₄ active)

Work up: Reaction mixture was quenched with cold water (500 mL) and extracted with EtOAc (300 mL×3). Combined organic layers dried over Na₂SO₄, concentrated under reduced pressure at 45° C. Crude was purified through combiflash at 2-3% EtOAc/Hexane to afford Int-7 as oily liquid.

Yield: 10.72 g, (98.3%); Characterized by ¹H-NMR

### Step-7: Synthesis of Heptanedioic acid mono-tert-butyl ester (Int-12)

To a stirred solution of Pimelic acid (Int-11) (30 g, 0.187 mol) in THF (300 mL, 10 V) and tert-butanol (150 mL, 5 V) at 0° C., Boc anhydride (40.8 g, 0.187 mol) was added dropwise followed by DMAP (11.49 g, 0.0936 mol) in a dropwise manner and slowly brought to ambient temperature. Resulting reaction was stirred for 18 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 45° C. Crude was purified through combiflash product eluted at 30% EtOAc/Hexane to afford Int-12 as oily liquid.

Yield: 16.87 g, (41.65%); Characterized by ¹H-NMR

### Step-8: Synthesis of 7-Hydroxy-heptanoic acid tert-butyl ester (Int-13)

To a stirred solution of Int-12 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 50% EtOAc in Hexane (KMnO4 active)

Work up: Reaction mixture was cooled to 0° C., quenched with MeOH (500 mL). Reaction mixture was concentrated under reduced pressure at 45° C. Crude was purified through combiflash, product eluted at 24-25% EtOAc/Hexane to afford Int-13 as oily liquid.

Yield: 8.63 g, (46.18%); Characterized by ¹H-NMR

### Step-9: Synthesis of 2-Nonyl-undec-5-enoic acid 6-tert-butoxycarbonyl-hexyl ester (Int-14)

To a stirred solution of Int-8 (5.8 g, 0.0186 mol) in DCM (38.8 mL, 20 Vol.) was added Int-13 (3.77 g, 0.0186 mol) followed by addition of EDC·HCl (5.79 g, 0.0373 mol) and DMAP (1.14 g, 0.0093 mol) by maintaining temperature 5° C.-10° C. DIPEA (13 mL, 0.0384 mol) was added in a drop wise manner at same temperature. After complete addition, the reaction mixture was stirred at 25-30° C. for 42 h. Progress of the reaction was monitored by TLC. TLC shows new non polar spot formation.

TLC eluent: 40% EtOAc in Hexane (KMnO4 active)

Work up: Reaction mixture was quenched with 10% NaHCO₃ (300 mL) and extracted with DCM (300 mL×3). Combined organic layers were dried over anhydrous sodium sulphate and concentrated under reduced pressure at 45° C. Crude was purified through combiflash at 1-3% EtOAc/Hexane to afford Int-14 as pale-yellow liquid.

Yield: 5.5 g, (59.5%); Characterized by ¹H-NMR

### Step-10: Synthesis of 2-Nonyl-undec-5-enoic acid 6-carboxy-hexyl ester (Int-5)

A stirred solution of Int-14 (5.6 g, 0.0113 mol) in DCM (11.2 mL, 2 V) was cooled to at 0-10° C., Trifluoroaceticacid (44.8 mL, 8 V) was added in a dropwise manner at 0-5° C. Reaction mixture was stirred at 25-30° C. for 18 h. Progress of reaction monitored by TLC. TLC shows new polar spot formation.

TLC eluent: 70% EtOAc in Hexane (KMnO4 active)

Work up: Reaction mixture was evaporated to dryness under reduced pressure at 45° C. Residue was dissolved in DCM (1×100 ml), neutralized with 10% sodium bicarbonate solution (500 ml×2). Organic layer was dried over anhydrous sodium sulphate and concentrated under reduced pressure at 45° C. to afford Int-5 as pale brown liquid.

Yield: 4.5 g, (Crude); Characterized by ¹H-NMR

### Example 6: Synthesis of Lipid-6

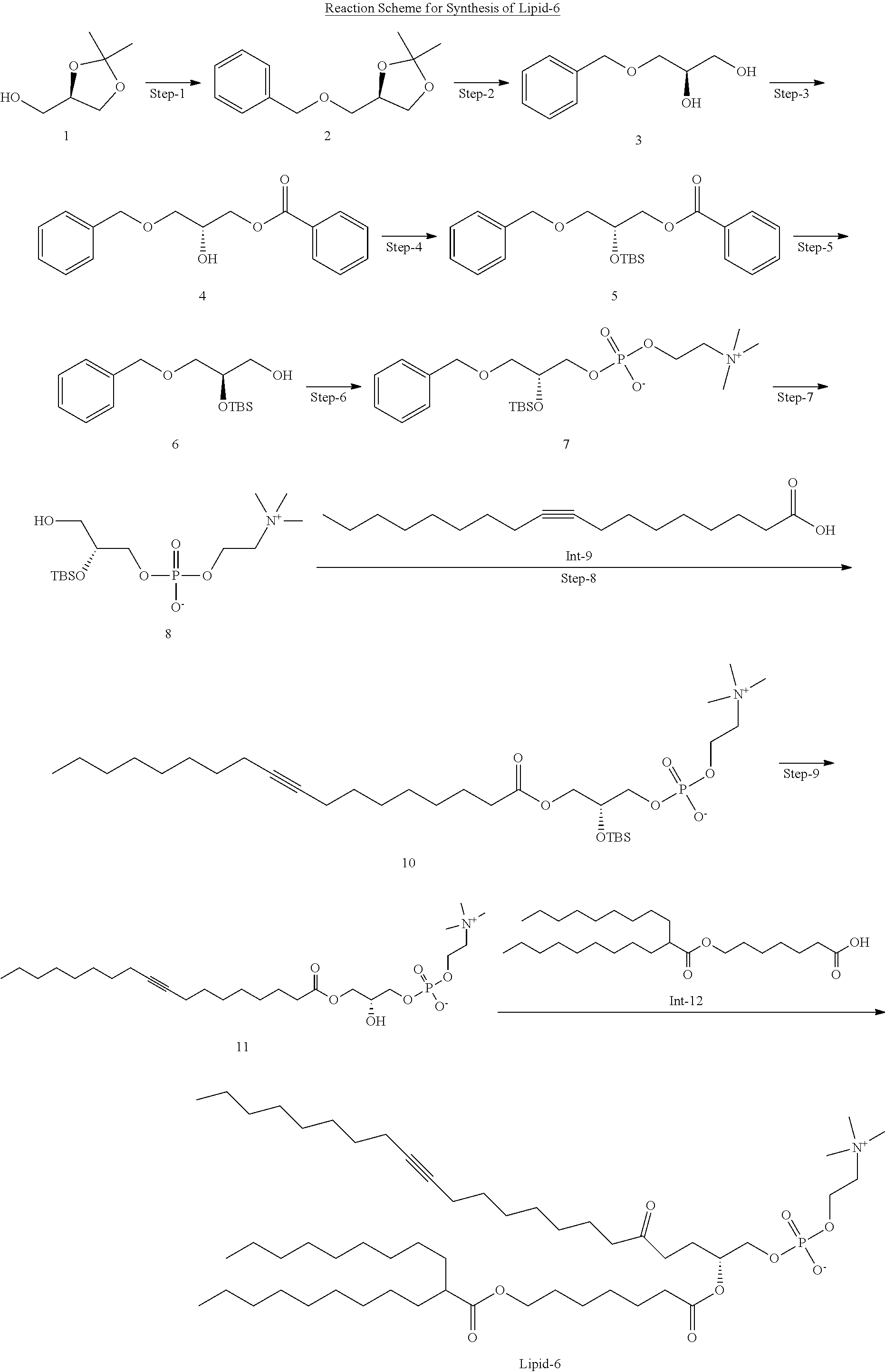

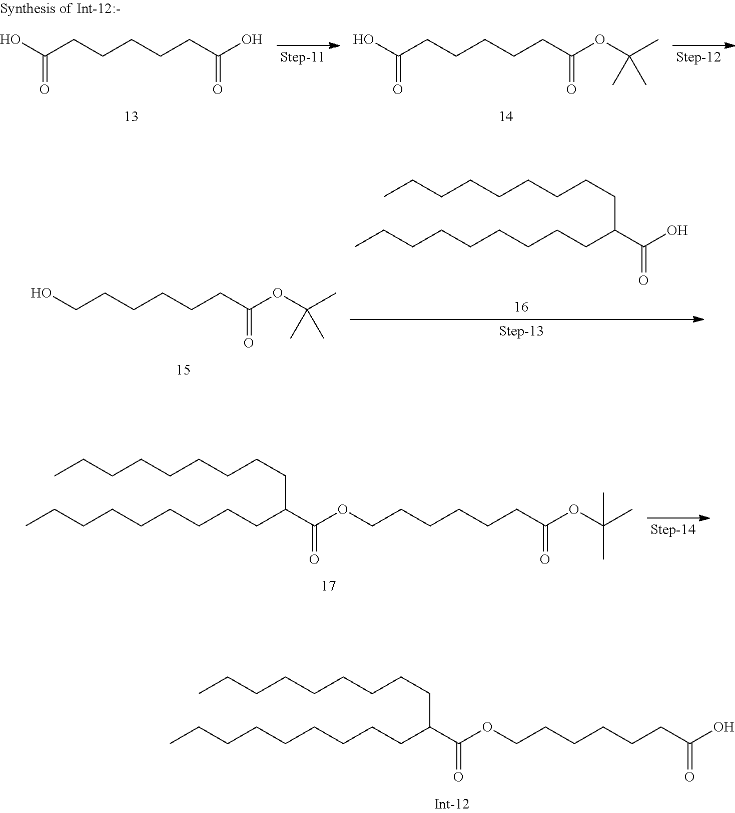

### Synthetic Procedure

### Step-1: Synthesis of 4-Benzyloxymethyl-2,2-dimethyl-[1,3]dioxolane (Int-2)

In a 3 neck RB (2,2-Dimethyl-[1,3]dioxolan-4-yl)-methanol (20 g, 0.0151 mol) was taken in THF (500 mL, 25V) and DMF (500 mL, 25V) and cooled for 0° C. Added NaH (60% in oil) (6.65 g, 0.1664 mol) dropwise (1.66 g×4 lots) over interval of 5 minutes, followed by Benzyl chloride (19.1 mL, 0.1664 mol). RM was brought to ambient temperature and stirred over a period of 18 h at 25-30° C. under argon atmosphere. Progress of the reaction was monitored by TLC. TLC shows new non-polar spot formation.

TLC eluent: 40% Ethyl acetate in hexane-KMnO₄ active

Work up: Reaction mass was cooled to 0° C. diluted with methanol (1000 mL) and stirred for 30 minutes at same temperature. RM was concentrated under reduced pressure. Obtained residue was diluted with water (800 mL) and extracted with DCM (750 mL×2). Combined organic layers were dried over anhydrous sodium sulphate and concentrated under reduced pressure at 45° C. to afford crude. Crude was purified through combiflash at 8-10% ethyl acetate-hexane to afford Int-2 as pale-yellow oily liquid.

Yield: 30.01 g (89.2%); Characterized by ¹H NMR

### Step-2: Synthesis of 3-Benzyloxy-propane-1,2-diol (Int-3)

To a solution of Int-2 (10 g, 0.045 mol) in methanol (50 mL, 5 V) and water (50 mL, 5 V) at 0-5° C., added DOWEX H⁺ resin (4 g, 40% w/w). Resulting reaction mixture was stirred for 18 h at 25-30° C. Progress of the reaction was monitored by TLC. TLC shows new polar spot formation.

TLC eluent: 50% Ethyl acetate in hexane-KMnO₄ active

Work up: Reaction mixture was filtered and obtained filtrate was concentrated under reduced pressure at 40° C. Residue was diluted with water (300 mL) and extracted with DCM (450 mL×2). Combined organic layers were dried over anhydrous sodium sulphate and concentrated under reduced pressure at 45° C. to afford Int-3 as pale brown oil.

Yield: 8.2 g (crude); Characterized by ¹H NMR

### Step-3: Synthesis of Benzoic acid 3-benzyloxy-2-hydroxy-propyl ester (Int-4)

To a stirred solution of Int-3 (8 g, 0.0439 mol) in ACN (200 mL, 25 V) added 2-diphenyl boronyloxyethanamine (0.988 g, 0.00439 mol), Diisopropylethylamine (11.46 mL, 0.0658 mol) followed by Benzoyl chloride (7.64 mL, 0.0658 mol) dropwise at ambient temperature. Resulting reaction mixture was stirred for 2 h at room temperature. Progress of the reaction was monitored by TLC. TLC shows new non-polar spot formation.

TLC eluent: 50% Ethyl acetate in hexane-KMnO₄ active

Work up: Reaction mixture was quenched with water (400 mL) and extracted with Ethyl acetate (250 mL×3), combined organic layers were dried over anhydrous sodium sulphate and concentrated under reduced pressure at 45° C. to afford Int-4.

Yield: 16.7 g (Crude); Characterized by ¹H NMR

### Step-4: Synthesis of Benzoic acid 3-benzyloxy-2-(tert-butyl-dimethyl-silanyloxy)-propyl ester (Int-5)

To a stirred solution of Int-4 (16.5 g, 0.0576 mol) in DMF (247.5 mL, 15 V) at 0° C. added Imidazole (11.76 g, 0.0864 mol) followed by Tert-butyldimethylsilylchloride in a dropwise manner. Reaction mixture was brought to ambient temperature and stirred for 18 h. Progress of the reaction was monitored by TLC. TLC shows new nonpolar spot formation.

TLC eluent: 10% EtOAc in Hexane (KMnO4 active)

Work up: Reaction mixture was quenched with ice-cold water (500 mL) and extracted with Ethyl acetate (300 mL×3), combined organic layers were dried over anhydrous sodium sulphate and concentrated under reduced pressure at 45° C. to afford crude. Crude was purified through combiflash at 5-6% EtOAC/Hexane to afford Int-5 as pale-yellow oily liquid.

Yield: 13.31 g (57.65%); Characterized by ¹H NMR

### Step-5: Synthesis of 3-Benzyloxy-2-(tert-butyl-dimethyl-silanyloxy)-propan-1-ol (Int-6)

To a stirred solution of Int-5 (12 g, 0.0299 mol) in THF (150 mL, 5 mL/mol) at room temperature added Ethyl magnesium bromide (105 mL, 0.104 mol) in a dropwise manner. Reaction mixture stirred for 2 h at ambient temperature. Progress of the reaction was monitored by TLC. TLC shows new polar spot formation.

TLC eluent: 40% in Ethyl acetate in Hexane-KMnO₄ active

Work up:—Reaction mass was cooled to −5 to 0° C., quenched with the addition of cold saturated NH₄Cl solution (1000 mL) by maintaining −5 to 0° C. The resultant reaction mixture was warmed to room temperature and extracted with ethyl acetate (500 mL×3). Organic layer was dried over anhydrous sodium sulphate and concentrated under reduced pressure at 45° C. to afford crude. Crude was purified through combiflash at 8-9% ethyl acetate-hexane to afford Int-6 as pale-yellow oily liquid.

Yield: 8 g (90%); Characterized by ¹H NMR

### Step-6: Synthesis of Int-7

To a stirred solution of Int-6 (2 g, 0.00674 mol) in DCM (16 mL, 8 V) at 0° C. added DMAP (82.4 mg, 0.000674 mol) followed by Triethylamine (9.44 mL, 0.0674 mol) in a dropwise manner, added 2-Chloro-1,3,2-dioxaphospholane (2.88, 0.0202 mol) dissolved in DCM (16 mL, 8 V) in a dropwise manner, after completion of addition reaction mixture was stirred at 0° C. for 60 minutes and later slowly brought to ambient temperature and stirred for 20 h. Progress of the reaction was monitored by TLC. TLC shows new spot formation Reaction mixture was concentrated under reduced pressure at 40° C. and obtained residue was diluted with THF (40 mL) and filtered through celite bed, obtained filtrate was concentrated under reduced pressure at 40° C. and dried over high vacuum for 60 minutes, obtained crude was diluted with ACN (20 mL, 15V) and transferred to autoclave vessel and cooled to −60 C°. In another RB Trimethylamine gas (9.96 g, 25 eq) was purged to 30 mL of ACN at −60° C. and added to previously cooled autoclave vessel containing crude at −60° C. Reaction mixture slowly brought to ambient temperature and heated to 65° C. for 18 h. Progress of the reaction was monitored by TLC. TLC shows new spot formation.

TLC eluent: 40% Methanol in DCM, 1 mL Aq NH₃ (KMnO4 and molybdenum blue active)

Work up: Reaction mixture was concentrated under reduced pressure at 40° C. Crude was purified by column manually at 10% Methanol in DCM, 1 mL aqueous NH₃ in silica to afford Int-7 as pale brown sticky solid.

Yield: 0.966 g (34.4%); Characterized by ¹H-NMR and LCMS

### Step-7: Synthesis of Int-8

A stirred solution of Int-7 (2.8 g, 0.00588 mol) in Methanol (112 mL, 40 V) was degassed for 10 minutes with argon, added 10% Pd/C (1.31 g, 47% w/w). Reaction mixture was heated to 35° C. with 1 kg/cm² pressure for 24 h. Progress of reaction monitored by TLC. TLC shows new polar spot formation.

TLC eluent: 60% Methanol in DCM, 2 mL Aq NH₃ (KMnO4 active)

Work up: Reaction mixture was filtered through celite bed and filtrate was evaporated to dryness under reduced pressure at 45° C. Crude was purified by column manually at 7-10% Methanol in DCM, 1 mL water in neutral alumina to afford Int-8 as off-white sticky solid.

Yield: 0.97 g (42.9%); Characterized by ¹H-NMR and LCMS

### Step-8: Synthesis of Int-10

To a stirred solution of Int-8 (0.8 g, 0.0021 mol) in DMF (20 mL, 25 V) was added DMAP (0.85 g, 0.0070 mol), DIC (1.49 mL, 0.0094 mol) and Int-9 (1.98 g, 0.0070 mol) at 25° C.-30° C. Resulting reaction mixture was stirred for 18 h at 35° C. Progress of the reaction was monitored by TLC. After completion of the reaction. Reaction mixture was directly concentrated under reduced pressure at 45° C. to get crude. Crude was purified through neutral alumina column using 0-15% MeOH/DCM and 1% H₂O to afford Int-10 as brown waxy solid.

Yield: 1.05 g (80%); Characterized by ¹H-NMR

### Step-09: Synthesis of Int-11

Int-10 (1.0 g, 0.0015 mol) was taken 2 neck RB, was added a solution of 0.1% TFA [(0.12 g, 0.0015 mol) in water (157 mL)]. Reaction mixture was stirred at 25-30° C. for 16 h. Progress of reaction monitored by TLC. TLC shows new polar spot formation

TLC eluent: 20% MeOH in DCM (KMnO₄ active & Molybdenum active) and 0.2 mL aq NH₃

Work up: Reaction mixture was lyophilized to afford Int-11 as off-white sticky solid.

Yield: 0.93 g, Crude; Characterized by ¹H-NMR and LCMS

LCMS: (EI, m/z) calcd for C₂₆H₅₀NO₇P [M+H]⁺: 521.5

### Step-10: Synthesis of Lipid-6

To a stirred solution of Int-11 (0.5 g, 0.0007 mol) in DCM (20 mL, 40 V) was added Int-12 (1.78 g, 0.0040 mol), DMAP (0.38 g, 0.0031 mol) and DIC (0.80 mL, 0.0064 mol) at 25° C.-30° C. Resulting reaction was stirred for 18 h at 25° C.-30° C. Progress of the reaction was monitored by TLC. After completion of reaction, reaction mixture was directly concentrated under reduced pressure at 45° C. to get crude. Crude was purified through neutral alumina column using CH₂Cl₂:MeOH:H2O (65/25/4) to afford BMS-L198 along with traces of DMAP. Impure BMS-L198 was dissolved in DCM (10 mL) added Dowex resin and stirred at 25-30° C. to remove DMAP. After 18 h, RM was filtered off and filtrate was evaporated at 45° C. to get pure Lipid-6 as pale brown waxy solid.

Yield: 0.45 g (60.08%); Characterized by ¹H-NMR and Q-TOF LCMS (Q-TOF): (EI, m/z) calcd for C₅₃H₁₁₀NO₁₀P: 942.7; HPLC (CAD) Purity: 61.6%

¹H NMR (400 MHz, MeOD): 5.26-5.23 (1H, m), 4.46-4.42 (1H, dd, J=12 Hz), 4.29 (2H, m), 4.17-4.14 (1H, m), 4.1 (2H, t, J=6.4 Hz), 4.0 (2H, t, J=5.6 Hz), 3.66-3.64 (2H, m), 3.23 (9H, s), 2.38-2.30 (5H, m), 2.12 (4H, m), 1.65-1.55 (8H, m), 1.42-1.40 (14H, m), 1.39-1.29 (40H, m), 0.9 (9H, m);

### Step-11: Synthesis of Heptanedioic acid mono-tert-butyl ester (Int-14)

To a stirred solution of Pimelic acid (Int-13) (25 g, 0.1560 mol) in THF (250 mL, 10 V) and tert-butanol (125 mL, 5 V) was added Boc anhydride (35.85 mL, 0.1560 mol) at 0-5° C. followed by DMAP (9.53 g, 0.0780 mol) in two equal lots. Resulting reaction was allowed to attain 25-30° C. and stirred for 18 h at same temperature. Progress of the reaction was monitored by TLC. After completion of reaction, reaction mixture was concentrated under reduced pressure at 45° C. to get crude. Crude was purified through combiflash®, compound was eluted at 0-1% MeOH/DCM to afford Heptanedioic acid mono-tert-butyl ester (Int-14) as colourless liquid.

Yield: 9.1 g (27%); Characterized by ¹H-NMR

### Step-12: Synthesis of 7-Hydroxy-heptanoic acid tert-butyl ester (Int-15)

To a stirred solution of Heptanedioic acid mono-tert-butyl ester (Int-14) (13 g, 0.060 mol) in THF (130 mL, 10 V) was added BH₃·DMS (10.9 mL, 0.1202 mol) in dropwise manner at 0-5° C. and slowly brought to ambient temperature. Resulting reaction was stirred for 18 h at room temperature. Progress of the reaction was monitored by TLC. After completion of reaction, reaction mixture was quenched by adding MeOH and concentrated under reduced pressure at 45° C. to get crude. Crude was purified through combiflash® to afford 7-Hydroxy-heptanoic acid tert-butyl ester (Int-15) as thick liquid.

Yield: 6 g (49.3%); Characterized by ¹H-NMR

### Step-13: Synthesis of 2-Nonyl-undecanoic acid 6-tert-butoxycarbonyl-hexyl ester (Int-17)

To a stirred solution of 2-nonylundecanoic Acid (Int-16) (1.1 g, 0.0054 mol) in DCM (22 mL, 20 V) was added Int-15 (1.70 g, 0.0054 mol) followed by EDC·HCl (2.08 g, 0.0108 mol) and DMAP (0.33 g, 0.0027 mol) DIPEA (3.71 mL, 0.0217 mol) were added at 0-5° C. After complete addition, the reaction mixture was stirred at 25-30° C. for 48 h. Progress of the reaction was monitored by TLC. After completion of reaction, reaction mixture was quenched by adding 10% aqueous NaHCO₃ solution (100 mL) and extracted with DCM (200 mL×2). Organic layer was dried over anhydrous sodium sulphate and concentrated under reduced pressure to get crude. Crude was purified through combiflash® to afford 2-Nonyl-undecanoic acid 6-tert-butoxycarbonyl-hexyl ester (Int-17) as colour less liquid.

Yield: 1.28 g (47.4%); Characterized by ¹H-NMR

### Step-14: Synthesis of 2-Nonyl-undecanoic acid 6-carboxy-hexyl ester (Int-12)

A stirred solution of 2-Nonyl-undecanoic acid 6-tert-butoxycarbonyl-hexyl ester (Int-17) (1.2 g, 0.0024 mol) in DCM (2.4 mL, 2 V) was added Trifluoroaceticacid (9.6 mL, 8 V) at 0-5° C. Reaction mixture was stirred at 25-30° C. for 18 h. Progress of reaction monitored by TLC. After completion of the reaction, reaction mixture was evaporated to remove excess of TFA. Residue was dissolved in DCM (30 ml) and neutralized with 10% sodium bicarbonate solution (40 ml). Organic layer separated, dried over anhydrous sodium sulphate and concentrated under reduced pressure at 45° C. to afford 2-Nonyl-undecanoic acid 6-carboxy-hexyl ester (Int-12) as pale brown liquid.

Yield: 1.02 g (Crude); Characterized by ¹H-NMR

### Example 7: Synthesis of Lipid-7

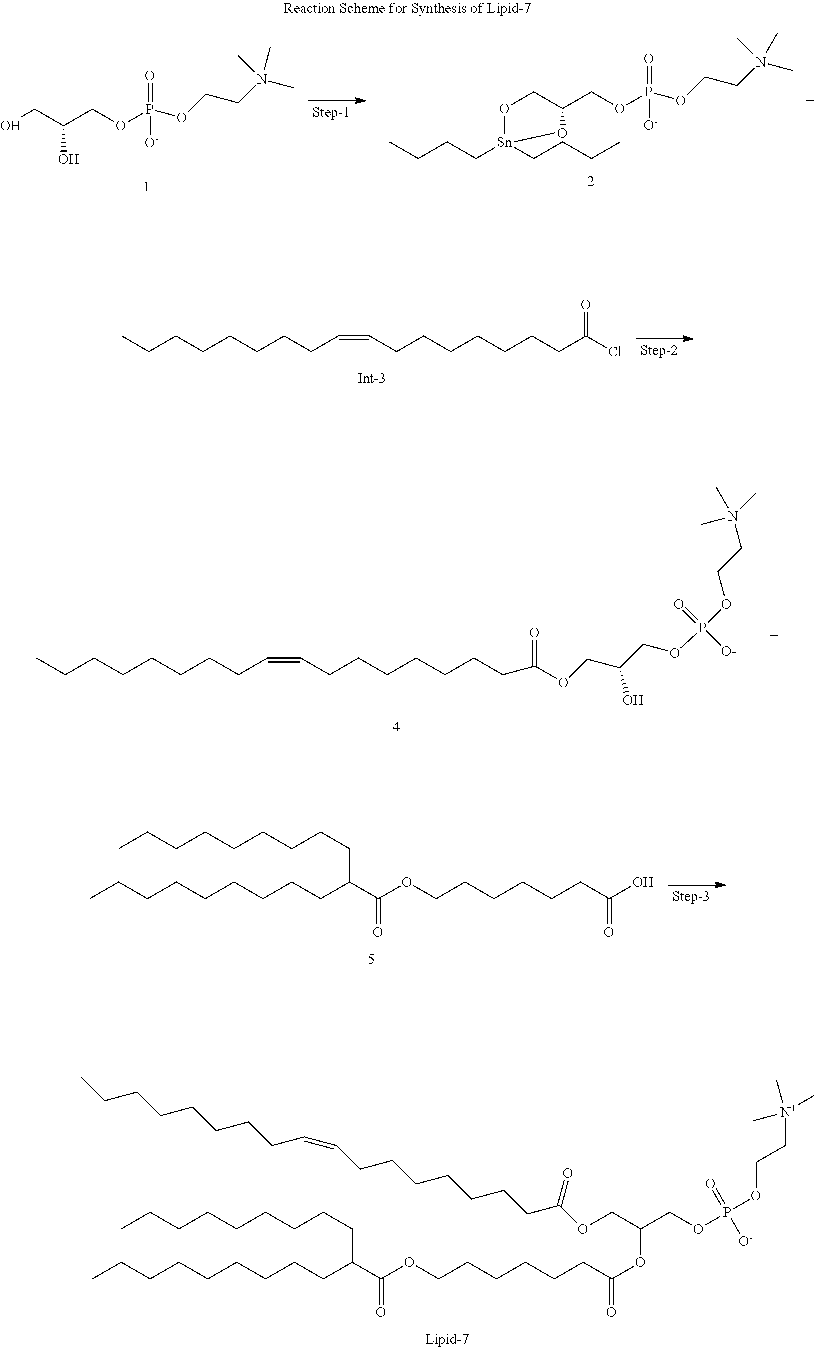

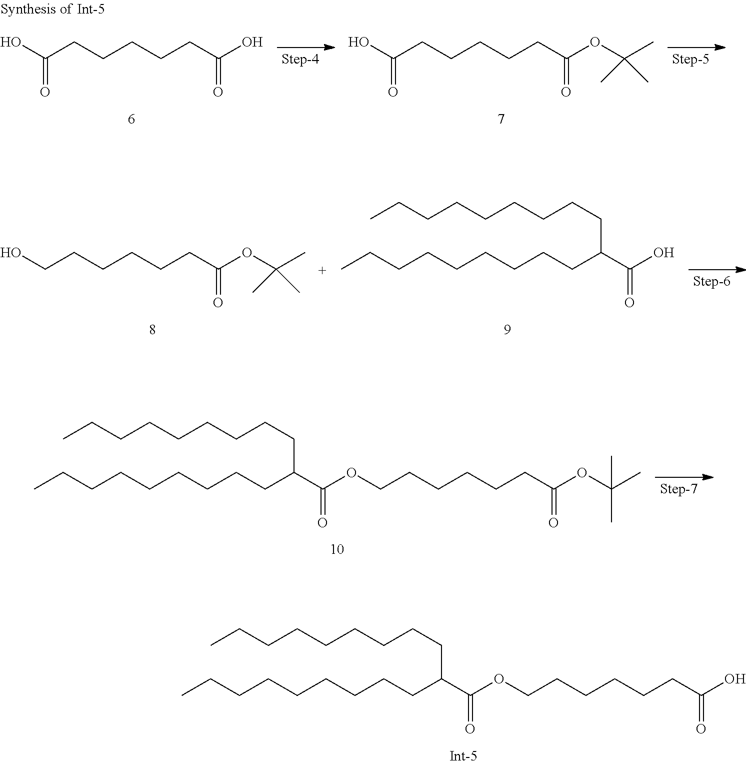

### Synthetic Procedure

### Step-1&2: Synthesis of Int-4

Choline glycerophosphate (2 g, 0.0077 mol) and dibutyltin oxide (DBTO) (2.12 g, 0.00854 mol) were suspended in 2-propanol (100 mL) and refluxed for 18 h at 90° C. The mixture was cooled to 0 to 5° C., TEA (1.3 mL, 0.00932 mol) and Int-3 (7.01 g, 0.0233 mol) were added. Resulting reaction mixture was stirred over a period of 36 h at 25-30° C. under argon atmosphere. Progress of the reaction was monitored by TLC. TLC shows new polar spot formation.

TLC eluent: (CH₂Cl₂/MeOH/Aq NH₃: 60/40/15)-KMnO₄ and Molybdenum blue active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Obtained crude was purified by column manually (20% MeOH/DCM & 1% H₂O) to get Int-4 as a white sticky solid.

Yield: 2.1 g (51.8%); Characterized by ¹H NMR and LCMS

### Step-3: Synthesis of Lipid-7

To a solution of Int-4 (0.75 g, 0.00137 mol) in DCM (37.5 mL, 50 V) at 0 to 5° C., were added Int-5 (0.95 g, 0.0021 mol), 2-methyl-6-nitrobenzoic anhydride [MNBA](2.55 g, 0.0732 mol) and DMAP (1.75 g, 0.0143 mol). Resulting reaction mixture was stirred for 48 h at 25-30° C. Progress of the reaction was monitored by TLC. TLC shows new non-polar spot formation.

TLC eluent: (IPA/EtOAc/H2O: 8/5/4) Molybdenum blue & KMnO₄ active

Work up: Reaction mixture was directly concentrated under reduced pressure at 40° C. Residue was dissolved in DCM and was purified manually using neutral alumina column chromatography with eluents (5% MeOH/DCM & 1% H₂O) to afford Lipid-7 as brown semi-solid.

Yield: 450 mg (33.3%); Characterized by ¹H NMR, ³¹P NMR, LCMS

LCMS: (EI, m/z) calcd for C₅₃H₁₀₂O₁₀P [M+H]⁺: 944.7; HPLC (CAD) purity: 82.44%

¹H NMR (400 MHz, CD₃OD): 5.35 (2H, t, J=8 Hz), 5.25-5.23 (1H, m), 4.46-4.42 (1H, dd, J=12 Hz), 4.27 (2H, m), 4.19-4.14 (1H, m), 4.084 (2H, t, J=6.4 Hz), 4.0 (2H, t, J=6 Hz), 3.6 (2H, t, J=4.4 Hz), 3.22 (9H, s) 2.37-2.3 (4H, m), 2.03 (4H, m), 1.65-1.55 (8H, m), 1.46-1.29 (56H, m), 0.91-0.85 (9H, m);

### Step-4: Synthesis of Heptanedioic acid mono-tert-butyl ester (Int-7)

To a stirred solution of Pimelic acid Int-6 (30 g, 0.187 mol) in THF (300 mL, 10 V) and tert-butanol (150 mL, 5 V) at 0° C., Boc anhydride (40.8 g, 0.187 mol) was added dropwise followed by DMAP (11.49 g, 0.0936 mol) in a dropwise manner and slowly brought to ambient temperature. Resulting reaction was stirred for 18 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 45° C. Crude was purified through combiflash at 30% EtOAc/Hexane to afford Int-7 as oily liquid.

Yield: 16.87 g, (41.65%); Characterized by ¹H NMR

### Step-5: Synthesis of 7-Hydroxy-heptanoic acid tert-butyl ester (Int-8)

To a stirred solution of Int-7 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 50% EtOAc in Hexane (KMnO4 active)

Work up: Reaction mixture was cooled to 0° C., quenched with MeOH (500 mL). Reaction mixture was directly concentrated under reduced pressure at 45° C. Crude was purified through combiflash at 24-25% EtOAc/Hexane to afford Int-8 as oily liquid.

Yield: 8.63 g (46.18%); Characterized by ¹H NMR

### Step-6: Synthesis of 2-Nonyl-undecanoic acid 6-tert-butoxycarbonyl-hexyl ester (Int-10)

To a stirred solution of Int-8 (1.94 g, 0.0096 mol) in DCM (38.8 mL, 20 Vol.) was added Int-9 (3 g, 0.0096 mol) followed by addition of EDC·HCl (2.98 g, 0.0192 mol) and DMAP (0.586 g, 0.0048 mol) by maintaining temperature 5° C.-10° C. DIPEA (6.6 mL, 0.0384 mol) was added in a drop wise manner at same temperature. After complete addition, the reaction mixture was stirred at 25-30° C. for 24 h. Progress of the reaction was monitored by TLC. TLC shows new non-polar spot formation.

TLC eluent: 30% EtOAc in Hexane (KMnO4 active)

Work up: Reaction mixture was quenched with 10% NaHCO₃ (300 mL) and extracted with DCM (200 mL×3). Combined organic layers were dried over anhydrous sodium sulphate and concentrated under reduced pressure at 45° C. Crude was purified through combiflash at 6-7% EtOAc/Hexane to afford Int-10 as pale-yellow liquid.

Yield: 3.6 g (75.6%); Characterized by ¹H NMR

### Step-7: Synthesis of 2-Nonyl-undecanoic acid 6-carboxy-hexyl ester (Int-5)

A stirred solution of Int-10 (3.5 g, 0.00704 mol) in DCM (7 mL, 2 V) was cooled to at 0-10° C., Trifluoroaceticacid (28 mL, 8 V) was added in a dropwise manner at 0-5° C. Reaction mixture was stirred at 25-30° C. for 18 h. Progress of reaction monitored by TLC. TLC shows new polar spot formation.

TLC eluent: 70% EtOAc in Hexane (KMnO4 active)

Work up: Reaction mixture was evaporated to dryness under reduced pressure at 45° C. Residue was dissolved in DCM (1×100 ml), neutralized with 10% sodium bicarbonate solution (100 ml×2). Organic layer was dried over anhydrous sodium sulphate and concentrated under reduced pressure at 45° C. to afford Int-5 as pale brown liquid.

Yield: 3 g (Crude); Characterized by ¹H NMR

### Example 8: Synthesis of Lipid-8

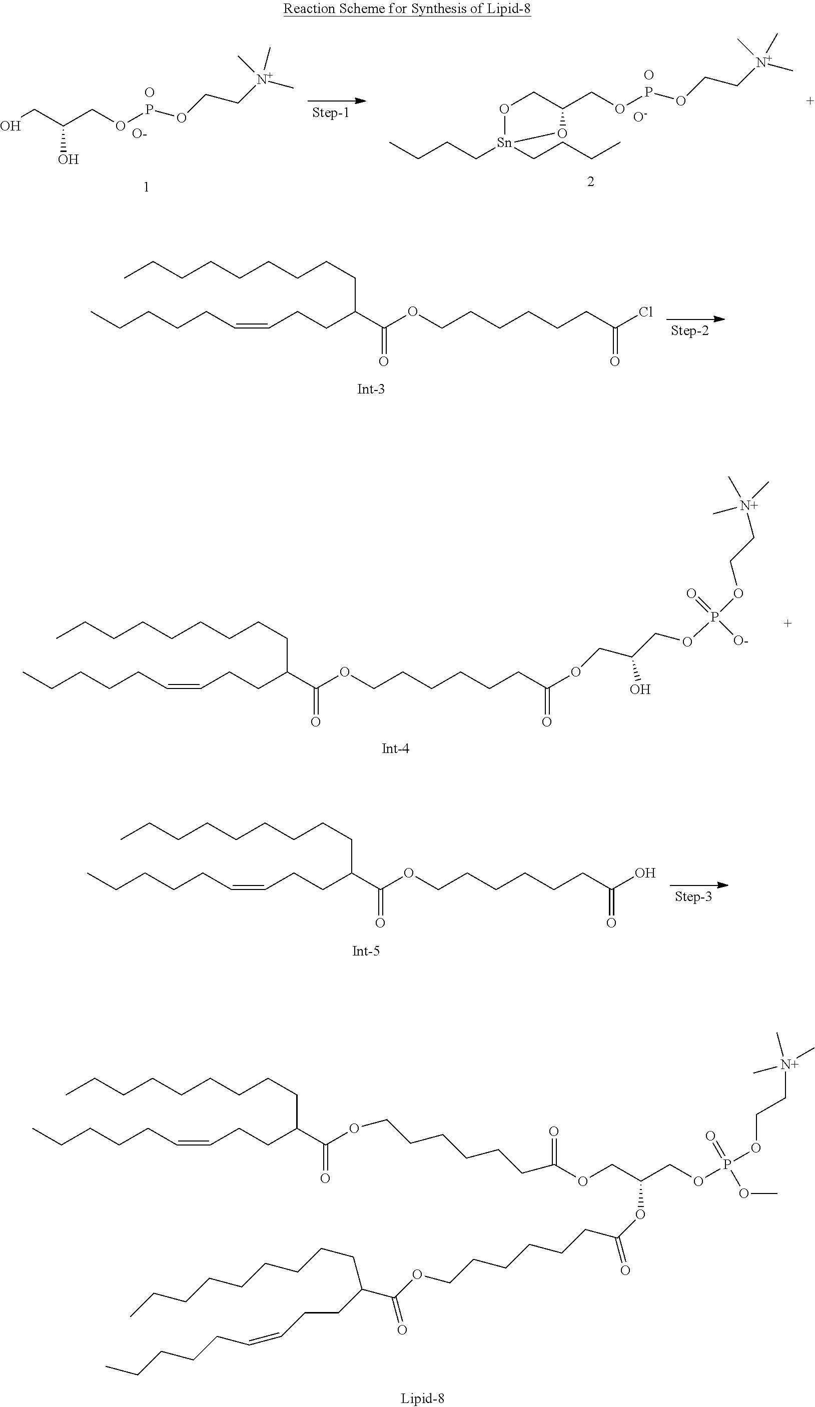

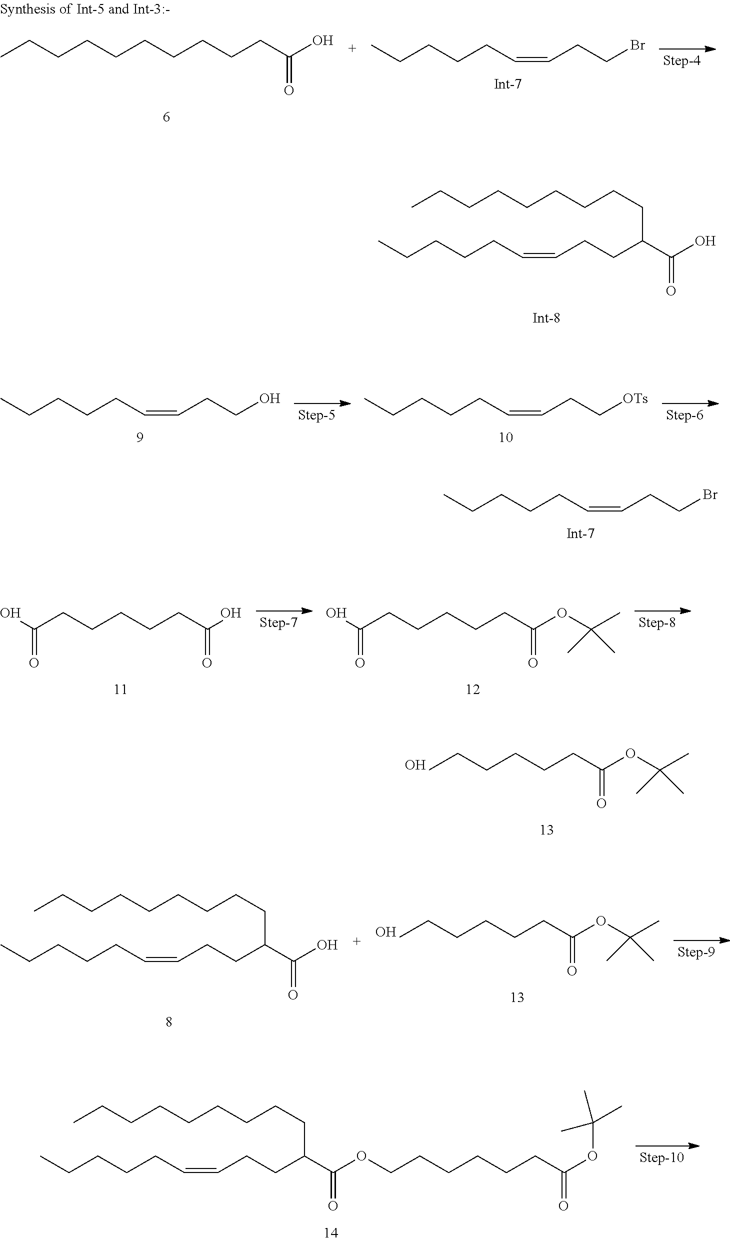

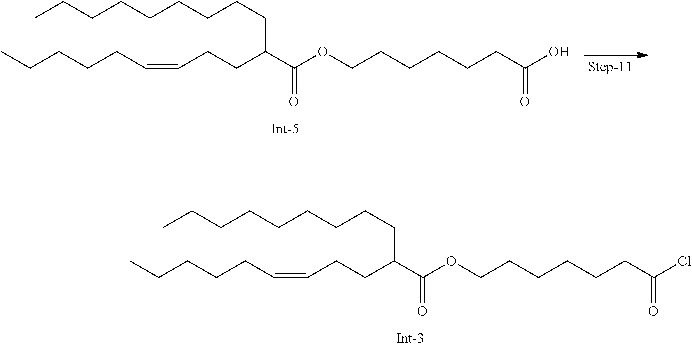 [SMILES: CCCCC/C=C\CCC(CCCCCCCCC)C(=O)OCCCCCCC(=O)O ; CCCCC/C=C\CCC(CCCCCCCCC)C(=O)OCCCCCCC(=O)Cl]

### Synthetic Procedure

### Step-1&2: Synthesis of Int-4

Choline glycerophosphate (2 g, 0.0077 mol) and dibutyltin oxide (DBTO) (2.12 g, 0.00854 mol) were suspended in 2-propanol (100 mL) and refluxed for 18 h at 90° C. The mixture was cooled to 0 to 5° C., TEA (1.3 mL, 0.00932 mol) and Int-3 (g, 0.00932 mol) dissolved in DCM (5 mL) were added. Resulting reaction mixture was stirred over a period of 36 h at 25-30° C. under argon atmosphere. Progress of the reaction was monitored by TLC. TLC shows new polar spot formation.

TLC eluent: (CH₂Cl₂/MeOH/Aq NH₃: 60/40/15)-KMnO₄ and Molybdenum blue active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Obtained crude was purified by column manually (10% MeOH/DCM & 1% H₂O) to get (Int-4) as a pale brown sticky solid.

Yield: 1.9 g, (36.12%); Characterized by ¹H-NMR

### Step-3: Synthesis of Lipid-8

To a solution of Int-4 (1.5 g, 0.00221 mol) in DCM (75 mL, 50 V) at 0 to 5° C., were added Int-5 (1.16 g, 0.00265 mol), 2-methyl-6-nitrobenzoic anhydride [MNBA](3.88 g, 0.0112 mol) and DMAP (2.7 g, 0.0221 mol). Resulting reaction mixture was stirred for 48 h at 25-30° C. Progress of the reaction was monitored by TLC. TLC shows new non-polar spot formation.

TLC eluent: TLC eluent: (IPA/EtOAc/H2O: 8/5/4) Molybdenum blue & KMnO₄ active

Work up: Reaction mixture was directly concentrated under reduced pressure at 40° C. Residue was dissolved in DCM and was purified by flash column using neutral alumina with eluents (5% MeOH/DCM & 1% H₂O) to afford Lipid-8 as pale-yellow waxy solid.

Yield: 850 mg, 34.97%; Characterized by ¹H-NMR and Q-TOF; HPLC (CAD) Purity: 73.53%

Q-TOF: (EI, m/z) calcd for C₆₂H₁₁₆O₁₂P [M+H]⁺: 1098.89

¹H NMR (400 MHz, CD₃OD): 5.38-5.25 (5H, m), 4.44 (1H, d, J=12 Hz), 4.27 (2H, s), 4.17 (1H, t, J=6.8 Hz), 4.09 (4H, m), 4.0 (2H, d, J=3.6 Hz), 3.64 (2H, s), 3.23 (9H, s) 2.35 (6H, m), 2.02 (8H, m), 1.64-1.54 (12H, m), 1.48-1.29 (52H, m), 0.90 (12H, m);

### Step-4: Synthesis of 2-Nonyl-undec-5-enoic Acid (Int-8)

In a 3 neck RB, THF (45 mL, 5 V) was cooled to −5 to 0° C. and was added LDA (2M in THF) (43.47 mL, 0.0869 mol) in a drop wise manner by maintaining temperature −5 to 0 30° C. After completion of addition, was added a solution of Int-6 (9 g, 0.0483 mol) in THF (45 mL, 5V) dropwise, followed by HMPA (9.49 mL, 0.0545 mol) dropwise. RM was immediately brought to ambient temperature and stirred for 30 minutes. RM was once again cooled to 0 to −5° C. and added Int-7 (10.89 g, 0.0531 mol) in a single addition. RM was immediately brought to ambient temperature and stirred for 4 h at ambient temperature. RM was monitored through TLC. TLC shows nonpolar spot formation along with SM.

TLC eluent: 20% in EtOAc in Hexane

Work up: Reaction mass was cooled to −5-0° C., quenched with the addition of cold 1.5 N HCl (300 mL) by maintaining −5 to 0° C. The resultant reaction mixture was warmed to room temperature and extracted with ethyl acetate (500 mL×2). Organic layer was dried over anhydrous sodium sulphate and concentrated under reduced pressure at 45° C. to afford crude. Crude was purified through combiflash at 2-3% ethyl acetate-hexane to afford Int-8 as pale-yellow oily liquid.

Yield: 5.81 g, 38%; Characterized by ¹H-NMR

### Step-5: Synthesis of Toluene-4-sulfonic acid non-3-enyl ester (Int-10)

To a stirred solution of Int-9 (18 g, 0.126 mol) in DCM (180 mL, 10 V) at 0° C. added TEA (61.4 mL, 0.441 mol) dropwise followed by Tosyl chloride (48.04 g, 0.252 mol) in a dropwise manner and slowly brought to ambient temperature. Resulting reaction was stirred for 18 h at room temperature. Progress of the reaction was monitored by TLC. TLC shows new nonpolar spot formation along with unreacted Tosyl chloride.

TLC eluent: 20% EtOAc in hexane (KMnO₄ active)

Work up: Reaction mixture was quenched with cold water (500 mL) and extracted with EtOAc (300 mL×3). Combined organic layers dried over Na₂SO₄, concentrated under reduced pressure at 45° C. Crude was purified through combiflash at 2-3% EtOAc/Hexane to afford Int-10 as oily liquid.

Yield: 20 g, (Impure); Characterized by ¹H-NMR

### Step-6: Synthesis of 1-Bromo-non-3-ene (Int-7)

To a stirred solution of Int-10 (15 g, 0.0523 mol) in THF (150 mL, 10 V) at ambient temperature, added magnesium bromide ethyl etherate (14.87 g, 0.0576 mol) dropwise, RM was heated to 40° C. for 16 h. Progress of the reaction was monitored by TLC. TLC shows new nonpolar spot formation.

TLC eluent: 10% EtOAc in hexane (KMnO₄ active)

Work up: Reaction mixture was quenched with cold water (500 mL) and extracted with EtOAc (300 mL×3). Combined organic layers dried over Na₂SO₄, concentrated under reduced pressure at 45° C. Crude was purified through combiflash at 2-3% EtOAc/Hexane to afford Int-7 as oily liquid.

Yield: 10.72 g, (98.3%); Characterized by ¹H-NMR

### Step-7: Synthesis of Heptanedioic acid mono-tert-butyl ester (Int-12)

To a stirred solution of Pimelic acid Int-11 (30 g, 0.187 mol) in THF (300 mL, 10 V) and to ambient temperature-butanol (150 mL, 5 V) at 0° C., Boc anhydride (40.8 g, 0.187 mol) was added dropwise followed by DMAP (11.49 g, 0.0936 mol) in a dropwise manner and slowly brought to ambient temperature. Resulting reaction was stirred for 18 h at room temperature. Progress of the reaction was monitored by TLC. TLC shows new non-polar spot formation.

TLC eluent: 10% MeOH in DCM (KMnO₄ active)

Work up: Reaction mixture was directly concentrated under reduced pressure at 45° C. Crude was purified through combiflash at 30% EtOAc/Hexane to afford Int-12 as oily liquid.

Yield: 16.87 g (41.65%); Characterized by ¹H-NMR

### Step-8: Synthesis of 7-Hydroxy-heptanoic acid tert-butyl ester (Int-13)

To a stirred solution of Int-12 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 50% EtOAc in Hexane (KMnO4 active)

Work up: Reaction mixture was cooled to 0° C., quenched with MeOH (500 mL). Reaction mixture was directly concentrated under reduced pressure at 45° C. Crude was purified through combiflash at 24-25% EtOAc/Hexane to afford Int-13 as oily liquid.

Yield: 8.63 g, (46.18%); Characterized by ¹H-NMR

### Step-9: Synthesis of 2-Nonyl-undec-5-enoic acid 6-tert-butoxycarbonyl-hexyl ester (Int-14)

To a stirred solution of Int-8 (5.8 g, 0.0186 mol) in DCM (38.8 mL, 20 Vol.) was added Int-13 (3.77 g, 0.0186 mol) followed by addition of EDC·HCl (5.79 g, 0.0373 mol) and DMAP (1.14 g, 0.0093 mol) by maintaining temperature 5° C.-10° C. DIPEA (13 mL, 0.0384 mol) was added in a drop wise manner at same temperature. After complete addition, the reaction mixture was stirred at 25-30° C. for 42 h. Progress of the reaction was monitored by TLC. TLC shows new non-polar spot formation.

TLC eluent: 40% EtOAc in Hexane (KMnO4 active)

Work up: Reaction mixture was quenched with 10% NaHCO₃ (300 mL) and extracted with DCM (300 mL×3). Combined organic layers were dried over anhydrous sodium sulphate and concentrated under reduced pressure at 45° C. Crude was purified through combiflash at 1-3% EtOAc/Hexane to afford Int-14 as pale-yellow liquid.

Yield: 5.5 g (59.5%); Characterized by ¹H-NMR

### Step-10: Synthesis of 2-Nonyl-undec-5-enoic acid 6-carboxy-hexyl ester (Int-5)

A stirred solution of Int-14 (5.6 g, 0.0113 mol) in DCM (11.2 mL, 2 V) was cooled to at 0-10° C., Trifluoroaceticacid (44.8 mL, 8 V) was added in a dropwise manner at 0-5° C. Reaction mixture was stirred at 25-30° C. for 18 h. Progress of reaction monitored by TLC. TLC shows new polar spot formation.

TLC eluent: 70% EtOAc in Hexane (KMnO4 active)

Work up: Reaction mixture was evaporated to dryness under reduced pressure at 45° C. Residue was dissolved in DCM (1×100 ml), neutralized with 10% sodium bicarbonate solution (500 ml×2). Organic layer was dried over anhydrous sodium sulphate and concentrated under reduced pressure at 45° C. to afford Int-5 as pale brown liquid.

Yield: 4.5 g (Crude); Characterized by ¹H-NMR

### Step-11: Synthesis of 2-Nonyl-undec-5-enoic acid 6-chlorocarbonyl-hexyl ester (Int-3)

A stirred solution of Int-5 (4.2 g, 0.0109 mol) in DCM (42 mL, 10 V) was cooled to at 0-10° C., Oxalyl chloride (1.87 mL, 0.0218 mol) was added in a dropwise manner at 0-5° C. followed by DMF (0.168 mL, 0.04 V). Reaction mixture was stirred at 25-30° C. for 5 h. Progress of reaction monitored by TLC. TLC shows new non-polar spot formation.

TLC eluent: 70% EtOAc in Hexane (KMnO4 active)

Work up: Reaction mixture was evaporated to dryness under reduced pressure and as such taken for next step.

Yield: 4.33 g (Crude)

### Example 9: Synthesis of Lipid-9

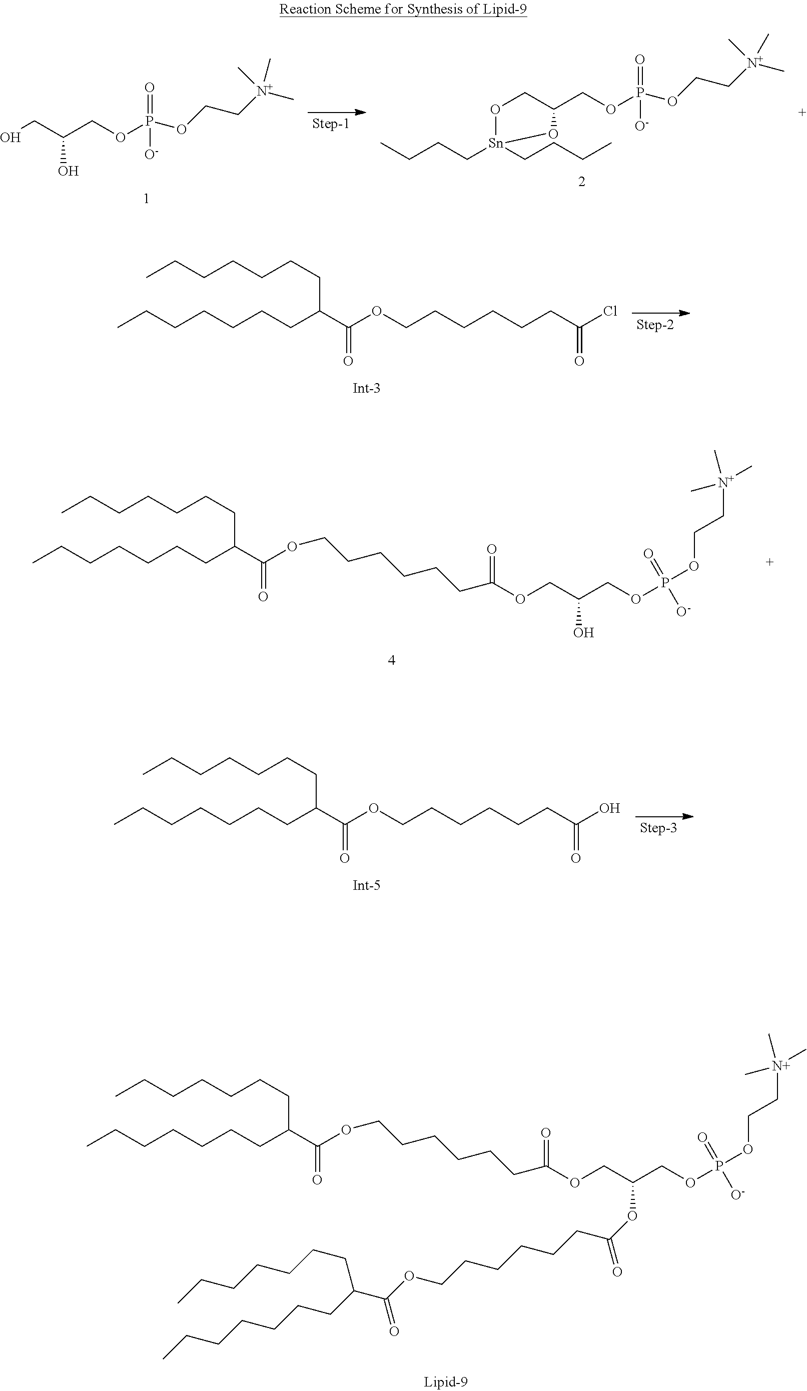

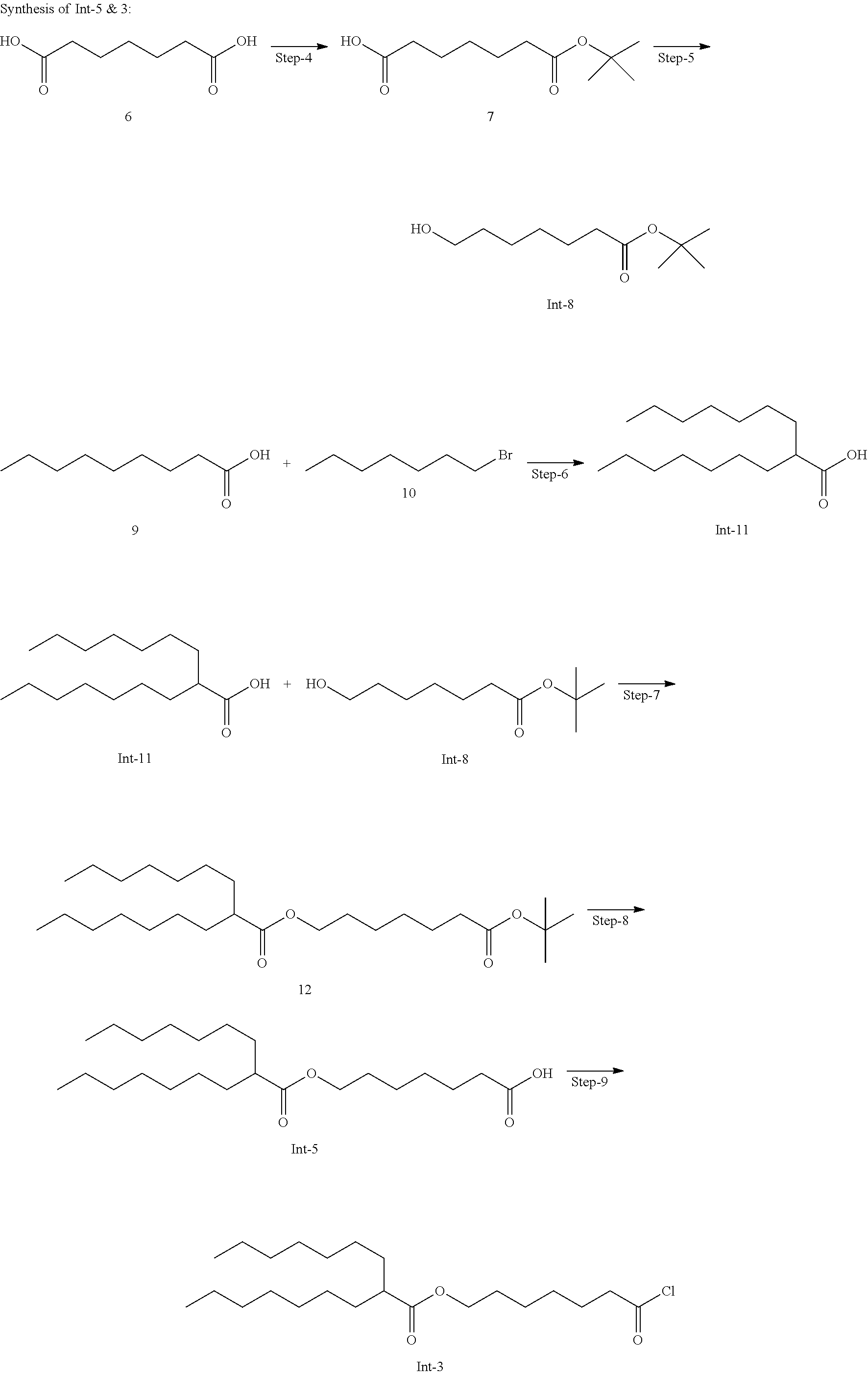

### Synthetic Procedure

### Step-1&2: Synthesis of Int-4

Choline glycerophosphate (2 g, 0.0077 mol) and dibutyltin oxide (DBTO) (2.12 g, 0.00854 mol) were suspended in 2-propanol (100 mL) and refluxed for 18 h at 90° C. The mixture was cooled to 0 to 5° C., TEA (1.3 mL, 0.00932 mol) and Int-3 (3.75 g, 0.0233 mol) were added. Resulting reaction mixture was stirred over a period of 36 h at 25-30° C. under argon atmosphere. Progress of the reaction was monitored by TLC. TLC shows new polar spot formation.

TLC eluent: (CH₂Cl₂/MeOH/Aq NH₃: 60/40/15)-KMnO₄ and Molybdenum blue active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Obtained crude was purified by column manually (20% MeOH/DCM & 1% H₂O) to get (Int-4) as off-white sticky solid.

Yield: 1.8 g, (37.19%); Characterized by ¹H-NMR

### Step-3: Synthesis of Lipid-9

To a solution of Int-4 (1.5 g, 0.0024 mol) in DCM (75 mL, 50 V) at 0 to 5° C., were added Int-5 (0.95 g, 0.0021 mol), 2-methyl-6-nitrobenzoic anhydride [MNBA](2.55 g, 0.0732 mol) and DMAP (1.75 g, 0.0143 mol). Resulting reaction mixture was stirred for 48 h at 25-30° C. Progress of the reaction was monitored by TLC. TLC shows new non-polar spot formation.

TLC eluent: TLC eluent: (IPA/EtOAc/H₂O: 8/5/4) Molybdenum blue & KMnO₄ active

Work up: Reaction mixture was directly concentrated under reduced pressure at 40° C. Residue was dissolved in DCM and was purified manually using neutral alumina column chromatography with eluents (5% MeOH/DCM & 1% H₂O) to afford Lipid-9 as pale green semi-solid.

Yield: 800 mg (33.61%); Characterized by ¹H-NMR and QTOF

Q-TOF: (EI, m/z) calcd for C₅₄H₁₀₄O₁₂P [M+H]⁺: 990.74; HPLC (CAD) Purity: 89.52%

¹H NMR (400 MHz, CD₃OD): 5.26-5.23 (1H, m), 4.46-4.42 (1H, dd, J=12 Hz), 4.27-4.25 (2H, m), 4.19-4.14 (1H, m), 4.08 (4H, t, J=6.4 Hz), 4.0 (2H, t, J=6 Hz), 3.65-3.63 (2H, m), 3.23 (9H, s), 2.38-2.3 (6H, m), 1.66-1.55 (12H, m), 1.49-1.38 (12H, m), 1.37-1.26 (40H, m), 0.90 (12H, t, J=7.2 Hz);

### Step-4: Synthesis of Heptanedioic acid mono-tert-butyl ester (Int-7)

To a stirred solution of Pimelic acid (Int-6) (30 g, 0.187 mol) in THF (300 mL, 10 V) and tert-butanol (150 mL, 5 V) at 0° C., Boc anhydride (40.8 g, 0.187 mol) was added dropwise followed by DMAP (11.49 g, 0.0936 mol) in a dropwise manner and slowly brought to ambient temperature. Resulting reaction was stirred for 18 h at room temperature. Progress of the reaction was monitored by TLC. TLC shows new nonpolar spot formation.

TLC eluent: 10% MeOH in DCM (KMnO₄ active)

Work up: Reaction mixture was directly concentrated under reduced pressure at 45° C. Crude was purified through combiflash at 30% EtOAc/Hexane to afford Int-7 as oily liquid.

Yield: 16.87 g (41.65%); Characterized by ¹H-NMR

### Step-5: Synthesis of 7-Hydroxy-heptanoic acid tert-butyl ester (Int-8)

To a stirred solution of Int-7 (20 g, 0.092 mol) in THF (200 mL, 10 V) at 0° C., BH₃·DMS (17.5 mL, 0.1850 mol) was added dropwise in a dropwise manner and slowly brought to ambient temperature. Resulting reaction was stirred for 18 h at room temperature. Progress of the reaction was monitored by TLC. TLC shows new spot formation.

TLC eluent: 50% EtOAc in Hexane (KMnO4 active)

Work up: Reaction mixture was cooled to 0° C., quenched with MeOH (500 mL). Reaction mixture was directly concentrated under reduced pressure at 45° C. Crude was purified through combiflash at 24-25% EtOAc/Hexane to afford Int-8 as oily liquid.

Yield: 8.63 g (46.18%); Characterized by ¹H-NMR

### Step-6: Synthesis of 2-Heptyl-nonanoic Acid (Int-11)

In a 3 neck RB, THF (50 mL, 10 V) was cooled to −5 to 0° C. and was added LDA (2M in THF) (56.9 mL, 0.113 mol) in a drop wise manner by maintaining temperature −5-0° C.

After completion of addition, a solution of Int-9 (10 g, 0.0632 mol) in THF (50 mL, 5V) was added dropwise, followed by HMPA (12.43 mL, 0.0714 mol) dropwise. RM was immediately brought to ambient temperature and stirred for 30 minutes. RM was once again cooled to 0 to −5° C. and added Int-10 (12.48 g, 0.069 mol) in a single addition. RM was immediately brought to ambient temperature and stirred for 4 h at RT. RM was monitored through TLC. TLC shows non-polar spot formation along with SM.

TLC eluent: 20% in EtOAc in Hexane

Work up: Reaction mass was cooled to −5-0° C., quenched with the addition of cold 1.5 N HCl (300 mL) by maintaining −5 to 0° C. The resultant reaction mixture was warmed to room temperature and extracted with ethyl acetate (500 mL×2). Organic layer was dried over anhydrous sodium sulphate and concentrated under reduced pressure at 45° C. to afford crude. Crude was purified through combiflash at 2-3% ethyl acetate-hexane to afford Int-11 as pale-yellow oily liquid.

Yield: 10 g (61.69%); Characterized by ¹H-NMR

### Step-7: Synthesis of 2-Heptyl-nonanoic acid 6-tert-butoxycarbonyl-hexyl ester (Int-12)

To a stirred solution of Int-11 (9.5 g, 0.037 mol) in DCM (190 mL, 20 V) was added Int-8 (7.49 g, 0.037 mol) followed by addition of EDC·HCl (11.5 g, 0.0741 mol) and DMAP (2.26 g, 0.0185 mol) by maintaining temperature 5° C.-10° C. DIPEA (25.8 mL, 0.148 mol) was added in a drop wise manner at same temperature. After complete addition, the reaction mixture was stirred at 25-30° C. for 24 h. Progress of the reaction was monitored by TLC. TLC shows new nonpolar spot formation.

TLC eluent: 20% EtOAc in Hexane (KMnO4 active)

Work up: Reaction mixture was quenched with 10% NaHCO₃ (500 mL) and extracted with DCM (300 mL×3). Combined organic layers were dried over anhydrous sodium sulphate and concentrated under reduced pressure at 45° C. Crude was purified through combiflash at 6-8% EtOAc/Hexane to afford Int-12 as pale brown liquid.

Yield: 8 g (49.01%); Characterized by ¹H-NMR

### Step-8: Synthesis of 2-Heptyl-nonanoic acid 6-carboxy-hexyl ester (Int-5)

A stirred solution of Int-12 (7.6 g, 0.0172 mol) in DCM (15.2 mL, 2 V) was cooled to at 0-10° C., Trifluoroaceticacid (60.8 mL, 8 V) was added in a dropwise manner at 0-5° C. Reaction mixture was stirred at 25-30° C. for 18 h. Progress of reaction monitored by TLC. TLC shows new polar spot formation.

TLC eluent: 20% EtOAc in Hexane (KMnO4 active)

Work up: Reaction mixture was evaporated to dryness under reduced pressure at 45° C. Residue was dissolved in DCM (1×100 ml), neutralized with 10% sodium bicarbonate solution (500 ml×2). Organic layer was dried over anhydrous sodium sulphate and concentrated under reduced pressure at 45° C. to afford crude which was purified through combiflash at 8-10% EtOAc/Hexane to afford Int-5 as pale brown liquid.

Yield: 7.01 g (Crude); Characterized by ¹H-NMR

### Step-9: Synthesis of 2-Heptyl-nonanoic acid 6-chlorocarbonyl-hexyl ester (Int-3)

A stirred solution of Int-5 (4 g, 0.0109 mol) in DCM (40 mL, 10 V) was cooled to at 0-10° C., Oxalyl chloride (1.78 mL, 0.0208 mol) was added in a dropwise manner at 0-5° C. followed by DMF (0.168 mL, 0.04 V). Reaction mixture was stirred at 25-30° C. for 5 h. Progress of reaction monitored by TLC. TLC shows new non-polar spot formation.

TLC eluent: 20% EtOAc in Hexane (KMnO4 active)

Work up: Reaction mixture was evaporated to dryness under reduced pressure and as such taken for next step.

Yield: 4.33 g (Crude) Example 10: Synthesis of Lipid-10

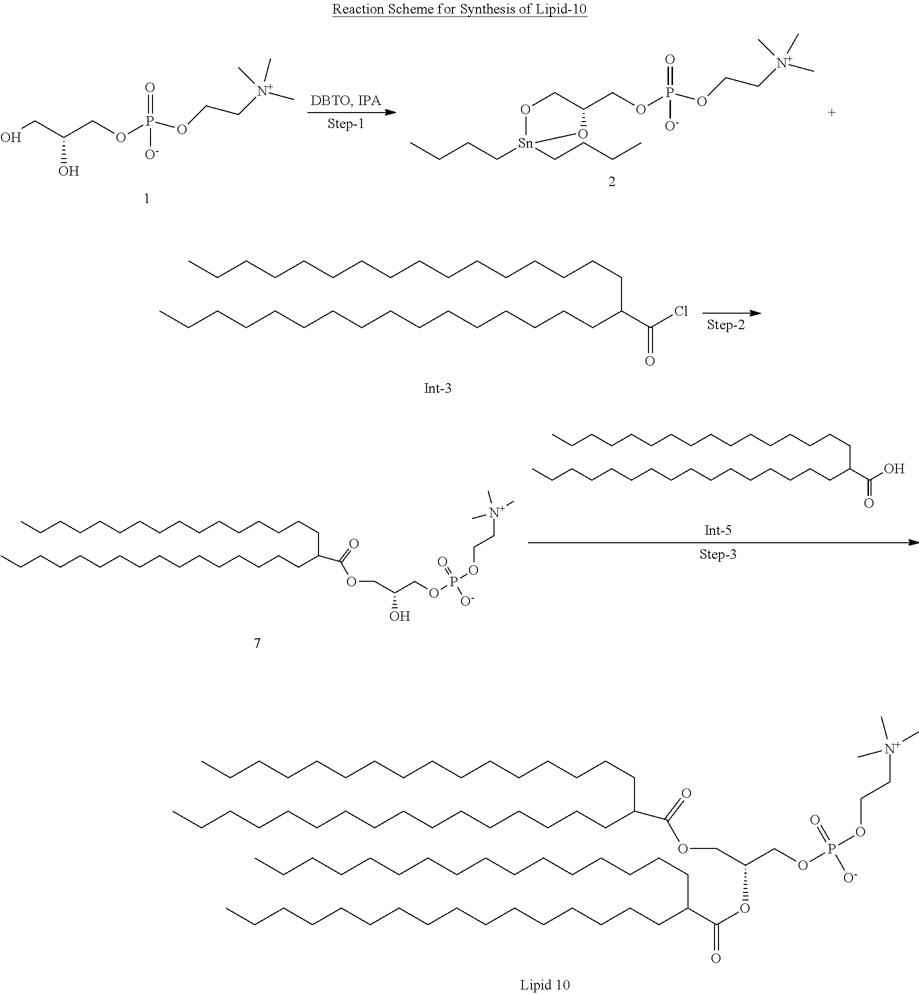

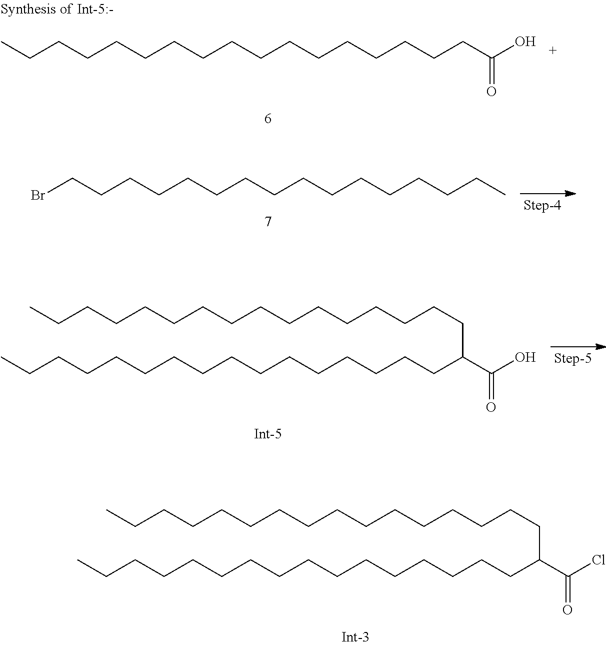

### Synthetic Procedure

### Step-1&2: Synthesis of Int-4

Choline glycerophosphate (Int-1) (2 g, 0.007 mol) and dibutyltin oxide (DBTO) (2.12 g, 0.0085 mol) were suspended in 2-propanol (100 mL) and refluxed at 90° C. for 18 h. The mixture was cooled to 0° C., TEA (1.3 mL, 0.0093 mol) and Int-3 (4.92 g, 0.0093 mol) were added at 0-5° C. Resulting reaction mixture was stirred over a period of 18 h at 25-30° C. under argon atmosphere. Progress of the reaction was monitored by TLC. After completion of the reaction, reaction mass was filtered through celite bed and bed was washed with 2-propanol (100 mL). Filtrate was collected and diluted with purified water (250 mL). The water-alcohol solution was extracted three times with heptane (3×250 mL) and resulting organic layer evaporated under reduced pressure at 45° C. to dryness to get crude. Crude was purified through neutral alumina column using CH₂Cl₂:MeOH:H₂O (65/25/4) to afford Int-4 as pale green waxy solid.

Yield: 2.3 g (39.6%); Characterized by ¹H-NMR

### Step-3: Synthesis of Lipid-10

To a stirred solution of Int-4 (2.3 g, 0.0030 mol) in DCM (115 mL, 50 V) was added Int-5 (2.34 g, 0.0043 mol) and 2-methyl-6-nitrobenzoic anhydride [MNBA](5.49 g, 0.0156 mol) and DMAP (3.75 g, 0.0307 mol) at 0-5° C. Resulting reaction mixture was stirred for 48 h at 25-30° C. Progress of the reaction was monitored by TLC. After completion of the reaction, reaction mixture was directly concentrated under reduced pressure at 40° C. Residue was treated with hexane (100 mL) and filtered off. Filtrate was evaporated at 45° C. to get crude and it was purified through neutral alumina column using CH₂Cl₂:MeOH:H₂O (65/25/4) to obtain partially pure material. Which was dissolved in DCM and added water (solid precipitates) then DCM was evaporated, suspension was filtered and dried under reduced pressure at 40° C. to afford Lipid-10 as pale brown waxy solid.

Yield: (0.35 g, 9.2%); Characterized by ¹H-NMR and Q-TOF; HPLC (CAD) purity: 80.1% LCMS (Q-Tof): (EI, m/z) calcd for C₇₆H₁₅₂NO₈P [M+H]: 1240.09

¹H NMR (400 MHz, CDCl₃): 5.20 (1H, m), 4.46-4.45 (1H, dd, J=12 Hz), 4.37-4.30 (2H, m), 4.26 (1H, m), 4.08-4.3.98 (1H, m), 3.91-3.90 (1H, m), 3.84 (2H, m), 3.38 (9H, s), 2.41-2.29 (2H, m), 1.66-1.53 (4H, m), 1.43-1.36 (4H, m), 1.32-1.02 (112H, m), 0.87 (12H, t, J=6.4 Hz)

### Step-4: Synthesis of Int-5

To A RBF containing THF (200 mL, 10 V) was added LDA 2M in THF (84.3 mL, 0.1689 mol), pre-dissolved solution of Int-6 (24 g, 0.0584 mol) in THF (40 mL) followed by HMPA (5.5 mL, 0.031 mol) slowly at 0° C. Reaction mixture was allowed to attain 25-20° C. and stirred at same temperature for 30 min. After 30 min, RM was cooled to 0° C. and added Int-7 (28.3 g, 0.0643 mol) rapidly. Reaction mixture was allowed to attain 25-30° C. and stirred for 2 h at same temperature. Progress of the reaction was monitored by TLC. After 2.5 h TLC shows formation of new spot along with Int-6. Reaction mixture was quenched by adding 1.5 M HCl (50 ml) at 0-5° C. and extracted with Ethyl acetate (2×300 ml).

Combined organic layer was dried over anhydrous Na₂SO₄ and concentrated to get the crude product which was purified by Combiflash using 0-2% of Ethyl acetate in Hexanes to afford Int-5 as off-white solid.

Yield: 12.9 g (30.1%); Characterized by ¹H-NMR

### Step-5: Synthesis of Int-3

A stirred solution of Int-5 (4.75 g, 0.0093 mol) in DCM (47.5 mL, 10 V) was added Oxalyl chloride (2.36 mL, 0.0186 mol) in a drop wise manner at 0-5° C. and catalytic DMF (0.1 mL) at same temperature. Resulting reaction was stirred for 3 h at room temperature. Progress of the reaction was monitored by TLC. After the completion of reaction, reaction mixture was concentrated under reduced pressure at 40° C. to dryness to afford Int-3 as yellow liquid. Crude was taken for next step immediately.

Yield: 5 g, Crude

### Example 11: Synthesis of Lipid-11

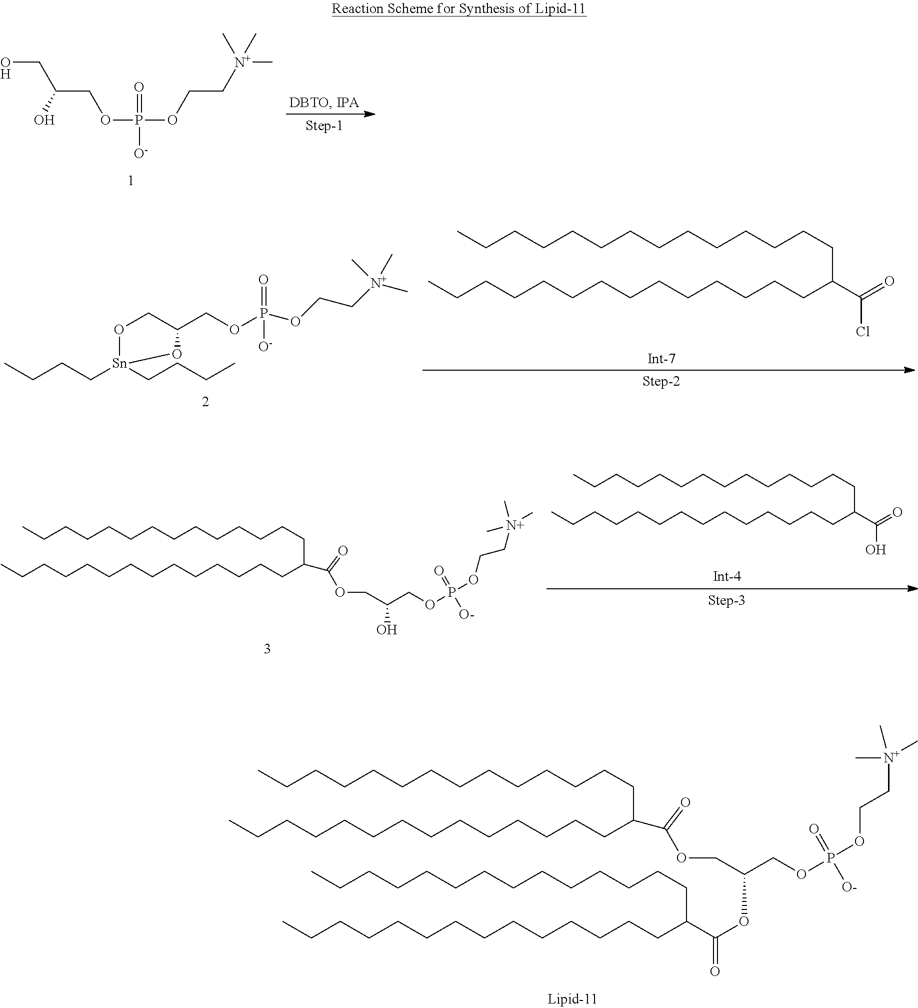

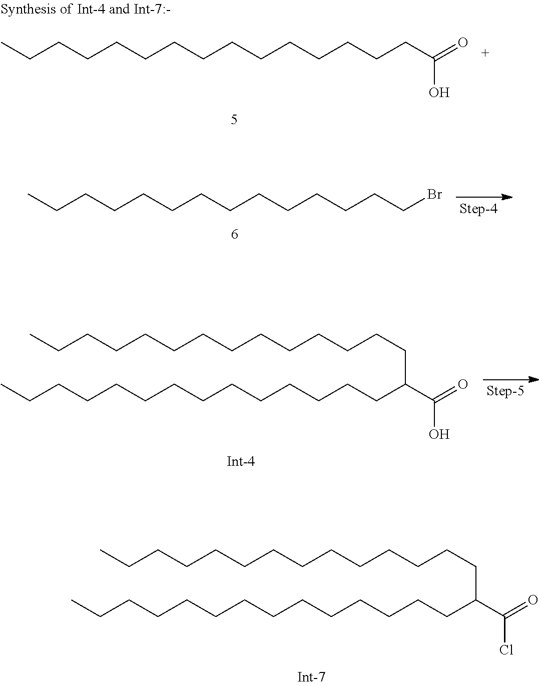 [SMILES: CCCCCCCCCCCCCCCC(=O)O ; CCCCCCCCCCCCCCBr ; CCCCCCCCCCCCCCC(CCCCCCCCCCCCCC)C(=O)O ; CCCCCCCCCCCCCCC(CCCCCCCCCCCCCC)C(=O)Cl]

### Synthetic Procedure

### Step-1&2: Synthesis of Int-3

Choline glycerophosphate (2 g, 0.077 mol) and dibutyltin oxide (DBTO) (2.12 g, 0.0085 mol) were suspended in 2-propanol (100 mL) and refluxed at 90° C. for 18 h. The mixture was cooled to 0° C. to 5° C., TEA (1.3 mL, 0.0093 mol) and Int-7 (4.39 g, 0.0093 mol) were added. Resulting reaction mixture was stirred over a period of 18 h at 25-30° C. under argon atmosphere. Progress of the reaction was monitored by TLC. After completion of the reaction, reaction mass was filtered through celite bed and bed was washed with 2-propanol (100 mL). Filtrate was collected and diluted with purified water (250 mL). The water-alcohol solution was extracted three times with heptane (3×250 mL) and resulting organic layer evaporated under reduced pressure at 45° C. to dryness to get crude. Crude was purified through neutral alumina column using CH₂Cl₂:MeOH:H₂O (65/25/4) to afford Int-3 as pale brown waxy solid.

Yield: 2.2 g, (42%); Characterized by ¹H-NMR

### Step-3: Synthesis of Lipid-11

To a stirred solution of Int-3 (2.0 g, 0.0028 mol) in DCM (100 mL, 50 V) was added Int-4 (1.96 g, 0.0043 mol), 2-methyl-6-nitrobenzoic anhydride [MNBA](5.07 g, 0.0147 mol) and DMAP (3.53 g, 0.0289 mol) at 0-5° C. Resulting reaction mixture was stirred for 48 h at 25-30° C. Progress of the reaction was monitored by TLC. After completion of the reaction, reaction mixture was directly concentrated under reduced pressure at 40° C. Residue was treated with hexane (100 mL) and filtered off. Filtrate was evaporated at 45° C. to get crude and it was purified through neutral alumina column using CH₂Cl₂:MeOH:H₂O (65/25/4) to obtain partially pure material, which was dissolved in DCM and added water (solid precipitated) then DCM was evaporated, the suspension was filtered. Solid was collected and dried under reduced pressure at 40° C. to afford Lipid-11 as grey waxy solid.

Yield: (0.42 g, 13.12%); Characterized by ¹H-NMR and Q-TOF; HPLC (CAD) purity: 94.94% LCMS (Q-TOF): (EI, m/z) calcd for C₆₈H₁₃₆NO₈P [M+H]: 1127.06 ¹H NMR (400 MHz, MeOD): 5.25-5.24 (1H, m), 4.61 (1H, dd, J=12 Hz), 4.29 (2H, s), 4.11-4.06 (1H, m), 4.01-3.97 (2H, m), 3.66-3.63 (2H, m), 2.37 (9H, s), 2.35 (2H, t, J=5.2 Hz), 1.61-1.47 (4H, m), 1.45-1.37 (4H, m), 1.29-1.24 (96H, m), 0.88 (12H, t, J=6.8 Hz);

### Step-4: Synthesis of Int-4

To A RBF containing THF (100 mL, 10 V) was added LDA 2M in THF (58.4 mL, 0.1169 mol), pre-dissolved solution of Int-5 (15.0 g, 0.0584 mol) in THF (50 mL) followed by HMPA (5 mL, 0.031 mol) were added slowly at 0° C. Reaction mixture was allowed to attain 25-30° C. and stirred at same temperature for 30 min. After 30 min, RM was cooled to 0° C. and added Int-6 (17.8 g, 0.0643 mol) rapidly. RM was allowed to attain 25-30° C. and stirred for 2 h at same temperature. Progress of the reaction was monitored by TLC.

After the completion of reaction, reaction mixture was quenched by adding 1.5 M HCl (50 ml) at 0-5° C. and extracted with Ethyl acetate (2×300 ml). Combined organic layer was dried over anhydrous Na₂SO₄ and concentrated to get the crude product, which was purified by Combiflash using 0-2% of Ethyl acetate in Hexanes to afford Int-4 as off-white solid.

Yield: 7.73 g (29.2%); Characterized by ¹H-NMR

### Step-5: Synthesis of Int-7

A stirred solution of Int-4 (4.3 g, 0.0094 mol) in DCM (43 mL, 10 V) was added oxalyl chloride (2.4 mL, 0.0189 mol) at 0-10° C. in a drop wise manner at 0-5° C. and catalytic DMF (0.1 mL) at same temperature. Resulting reaction was stirred for 3 h at room temperature. Progress of the reaction was monitored by TLC. After the completion of reaction, reaction mixture was concentrated under reduced pressure at 40° C. to dryness to afford Int-7 as yellow liquid. Crude was taken for next step immediately.

Yield: 5 g, (Crude)

### Example 12: Synthesis of Lipid-12

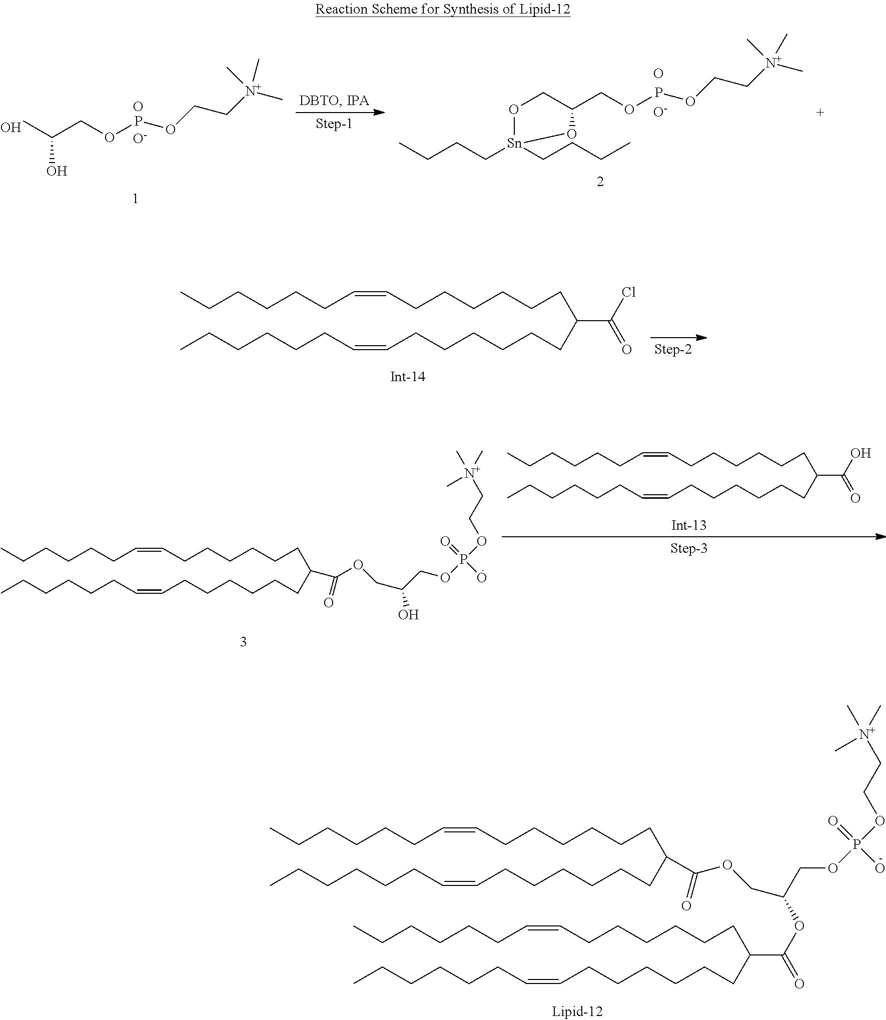

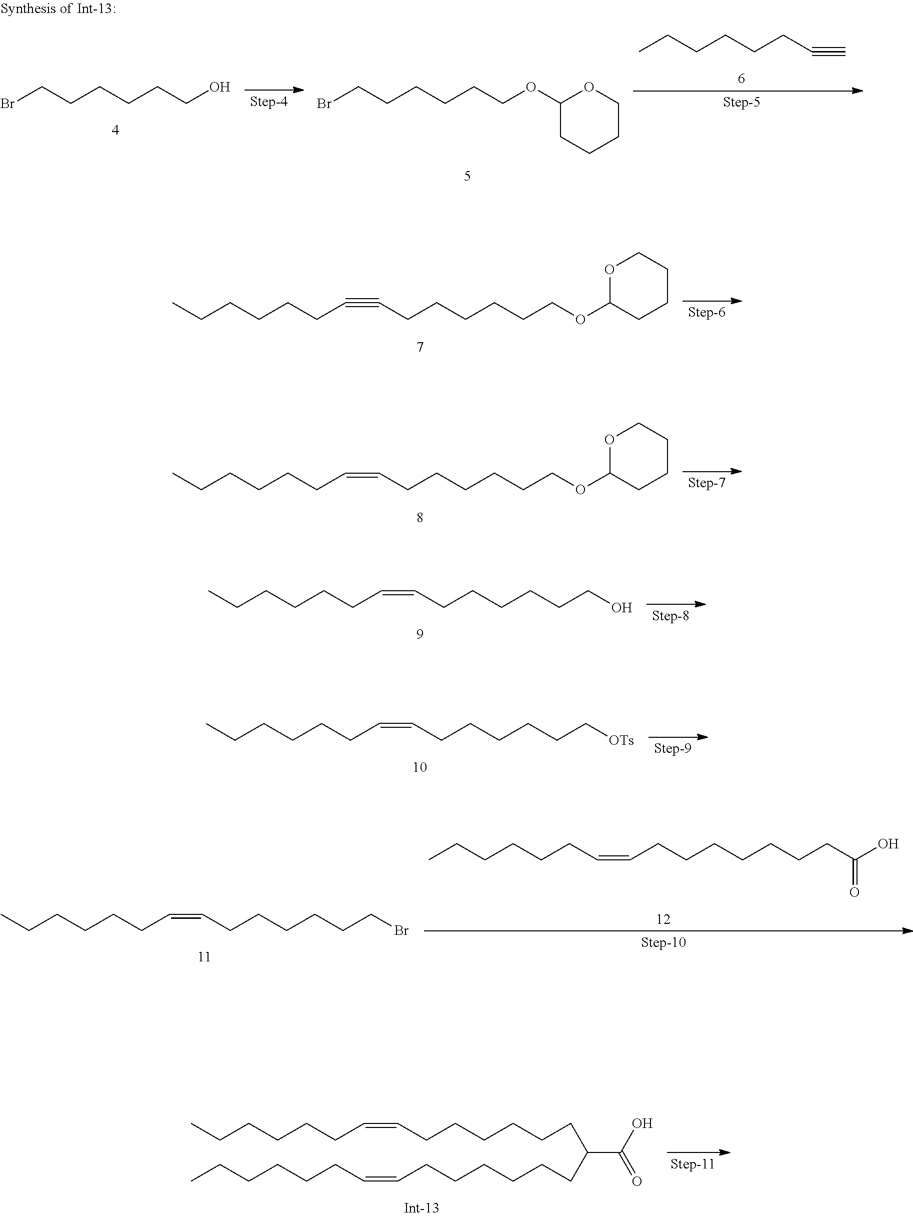

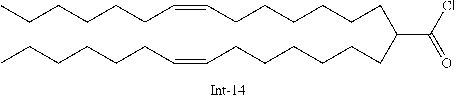 [SMILES: CCCCCC/C=C\CCCCCCC(CCCCCC/C=C\CCCCCC)C(=O)Cl]

### Synthetic Procedure

### Step-1&2: Synthesis of Int-3

Choline glycerophosphate (2 g, 0.077 mol) and dibutyltin oxide (DBTO) (2.12 g, 0.0085 mol) were suspended in 2-propanol (100 mL) and refluxed at 90° C. for 18 h. The mixture was cooled to 0° C. to 5° C., TEA (1.3 mL, 0.0093 mol) and Int-14 (4.39 g, 0.0093 mol) were added. Resulting reaction mixture was stirred over a period of 18 h at 25-30° C. under argon atmosphere. Progress of the reaction was monitored by TLC. After completion of the reaction, reaction mass was filtered through celite bed and bed was washed with 2-propanol (100 mL). Filtrate was collected and diluted with purified water (250 mL). The water-alcohol solution was extracted three times with heptane (3×250 mL) and resulting organic layer evaporated under reduced pressure at 45° C. to dryness to get crude. Crude was purified through neutral alumina column using CH₂Cl₂:MeOH:H₂O (65/25/4) to afford Int-3 as pale brown waxy solid.

Yield: 2.1 g (39.6%); Characterized by ¹H-NMR

### Step-3: Synthesis of Lipid-12

To a stirred solution of Int-3 (2.1 g, 0.00305 mol) and Int-13 (2.05 g, 0.00457 mol) in DCM (105 mL, 50 V) were added 2-methyl-6-nitrobenzoic anhydride (MNBA) (5.35 g, 0.015 mol) and DMAP (3.72 g, 0.030 mol) at 0-5° C. RM was allowed to attain 25-30° C. and stirred for 48 h. Progress of the reaction was monitored by TLC. After the completion of reaction, reaction mixture was evaporated completely to get crude. Crude was purified using flash column using neutral alumina to obtain partially purified BMS-L207 which was further purified in Combiflash using 8:2:1 (DCM:MeOH:water) to afford Lipid-12 as off-white semi solid.

Yield: 1 g (29.4%); Characterized by ¹H-NMR and Q-TOF LCMS (Q-TOF): (EI, m/z) calcd for C₆₈H₁₂₈NO₈P [M+H]: 1119; HPLC (CAD) Purity: 78.85% ¹H NMR (400 MHz, MeOD): 5.39-5.33 (8H, m), 5.30-5.24 (1H, m), 4.63-4.59 (1H, m), 4.28 (2H, m), 4.11-4.06 (1H, m), 4.06-3.96 (2H, m), 3.66-3.63 (2H, m), 3.23 (9H, s), 2.38-2.34 (2H, m), 2.04-1.99 (16H, m), 1.61-1.60 (4H, m), 1.47-1.45 (4H, m), 1.32-1.31 (64H, m), 0.91 (12H, t, J=6.4 Hz);

### Step-4: Synthesis of Int-5

To a stirred solution of Int-4 (35 g, 0.193 mol) in DCM (350 ml, 10 V) was added 3,4 dihydro-2H pyran (51.14 ml, 0.560 mol) at 0-5° C. followed by PTSA monohydrate (367 mg, 0.0199 mol) in two equal lots. Resulting reaction mixture was stirred at 25-30° C. for 5 h. Progress of reaction was monitored by TLC. After the completion of reaction, reaction mixture was quenched by adding 10% NaHCO₃ solution (200 ml) at 0-5° C. and extracted with DCM (2×200 ml). Combined organic layer was dried over anhydrous Na2SO4 and concentrated to get the crude product which was purified by Combi flash using 0-2.5% of Ethyl acetate in Hexanes to afford Int-5 as colour less thick liquid.

Yield: 21.3 g (48.8%); Characterized by ¹H-NMR

### Step-5: Synthesis of Int-7

To a stirred solution of Int-6 (24.3 g, 0.1351 mol) THF (239 ml, 10 V), was added HMPA (23.5 ml, 0.1351 mol) and n-BuLi 2.5M in THF (72 ml, 0.1802 mol) in a drop wise manner at −25 to −30° C. Resulting brown coloured reaction mass was stirred at −30° C. for 1 h. After 1 h, Int-5 (23.9 ml, 0.09012 mol) was added drop slowly at −25 to −30° C. Resulting reaction mixture was allowed to attain 25-30° C. and stirred at same temperature for 16 h. Progress of the reaction was monitored by TLC. After the completion of reaction, reaction mixture was quenched by adding 1.5 M HCl (200 ml) at 0-5° C. and extracted with Ethyl acetate (2×300 ml). Combined organic layer was dried over anhydrous Na₂SO₄ and concentrated to get the crude product, which was purified by Combi flash using 3-4% of Ethyl acetate in Hexanes to afford Int-7 as pale-yellow thick liquid.

Yield: 15.9 g, (60%); Characterized by ¹H-NMR

### Step-6: Synthesis of Int-8

To a stirred solution of Int-7 (8.2 g, 0.0272 mol) in Ethyl acetate (328 mL, 40 V), was degassed with argon for 10 min and added Lindlar's catalyst (412 mg, 5% w/w) and stirred under H₂ gas (Bladder) for 18 h at 25-30° C. Progress of the reaction was monitored by TLC. After 18 h, TLC shows formation of new complete consumption of Int-7. The reaction mixture was filtered through celite bed and bed was washed with ethyl acetate (500 mL). Filtrate was concentrated to get the Int-8 as pale-yellow thick liquid.

Yield: 8.17 g (Crude)

### Step-7: Synthesis of Int-9

To a stirred solution of Int-8 (8.1 g, 0.02731) in Methanol was added PTSA monohydrate (3.76 g, 0.02185 mol) in two equal lots at 0-5° C. Resulting reaction mixture was allowed to stir at 25-30° C. for 3 h. Progress of the reaction was monitored by TLC. After the completion of reaction, reaction mixture was quenched by adding solid NaHCO₃ (20 g) at 10-15° C. and filtered off. Filtrate was evaporated to get white residue which was treated with DCM (50 ml) and filtered off. Filtrate was evaporated to obtain Int-9 as thick liquid.

Yield: 5.5 g (94.8%); Characterized by ¹H-NMR

### Step-8: Synthesis of Int-10

To a stirred solution of Int-9 (5.5 g, 0.02589 mol) in DCM (55 ml, 10 V) was added TEA (12.6 mL, 0.09064 mol) resulting solution was allowed to stir at 0-5° C. for 15 min and added Tosyl chloride (16.3 g, 0.08546 mol) at same temperature. Resulting reaction mixture was allowed to stir at 25-30° C. for 24 h. Progress of the reaction was monitored by TLC. After the completion of reaction, reaction mixture was filtered off. Filtrate was evaporated to get crude, which was purified using 0-14% of Ethyl acetate in Hexanes to afford Int-10 as pale-yellow liquid Yield: 6.6 g (70%); Characterized by ¹H-NMR

### Step-9: Synthesis of Int-11

To a stirred solution of Int-10 (6.6 g, 0.01800 mol) in THF (66 ml, 10 V) was added Magnesium bromide ethyl etherate (13.4 g, 0.0522 mol) at 25-30° C. Resulting suspension was stirred at 40° C. for 18 h. After 18, TLC shows the complete consumption of Int-10. To the reaction mixture, water was added and extracted with ethyl acetate (100 mL×2). Combined organic layer was dried over anhydrous Na₂SO₄ and concentrated to get the crude which was purified by Combi flash. Product was eluted in plane Hexane to afford Int-11 as pale colourless thick liquid.

Yield: 4.7 g (87%); Characterized by ¹H-NMR

### Step-10: Synthesis of Int-13

To A RBF containing THF (30 mL) was added LDA 2M in THF (15.7 mL, 0.0314 mol), pre-dissolved solution of Int-12 (4 g, 0.0157 mol) in THF (10 mL) followed by HMPA (3.09 mL, 0.01776 mol) were added slowly at 0° C. RM was allowed to attain 25-30° C. and stirred at same temperature 30 min. After 30 min, RM was cooled to 0° C. and added Int-11 (4.76 g, 0.01729 mol) rapidly. RM was allowed to attain 25-30° C. and stirred for 2 h at same temperature. Progress of the reaction was monitored by TLC. After the completion of reaction, reaction mixture was quenched by adding 1.5 M HCl (100 ml) at 0-5° C. and extracted with Ethyl acetate (2×300 ml). Combined organic layer was dried over anhydrous Na2SO4 and concentrated to get the crude product which was purified by Combi flash using 0-1.5% of Ethyl acetate in Hexanes to afford Int-13 as pale-yellow thick liquid.

Yield: 3.2 g (45.7%); Characterized by ¹H-NMR

### Step-11: Synthesis of Int-14

A stirred solution of Int-13 (4.2 g, 0.0094 mol) in DCM (43 mL, 10 V) was added Oxalyl chloride (2.4 mL, 0.0189 mol) was added in a drop wise manner at 0-5° C. and catalytic DMF (0.1 mL) at same temperature. Resulting reaction was stirred for 3 h at room temperature. Progress of the reaction was monitored by TLC. After the completion of reaction, reaction mixture was concentrated under reduced pressure at 40° C. to dryness to afford Int-14 as yellow liquid. Crude was taken for next step immediately.

Yield: 5 g, (Crude)

### Example 13: Synthesis of Lipid-13

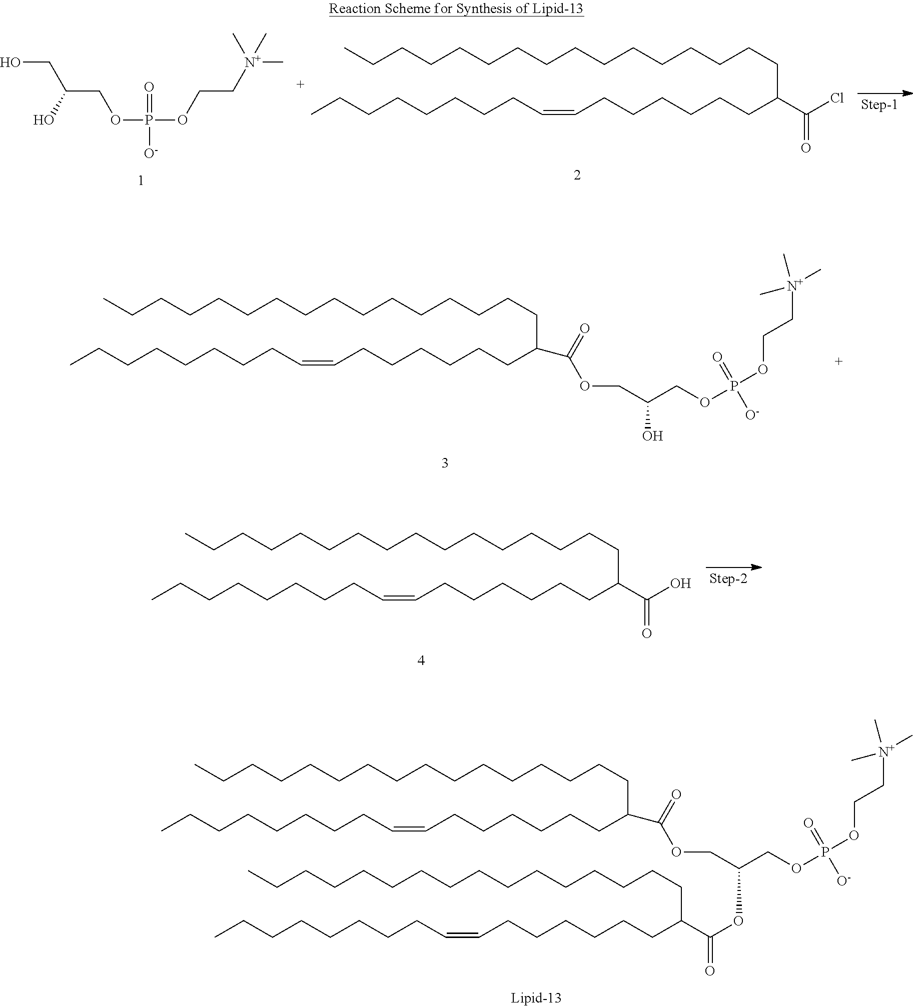

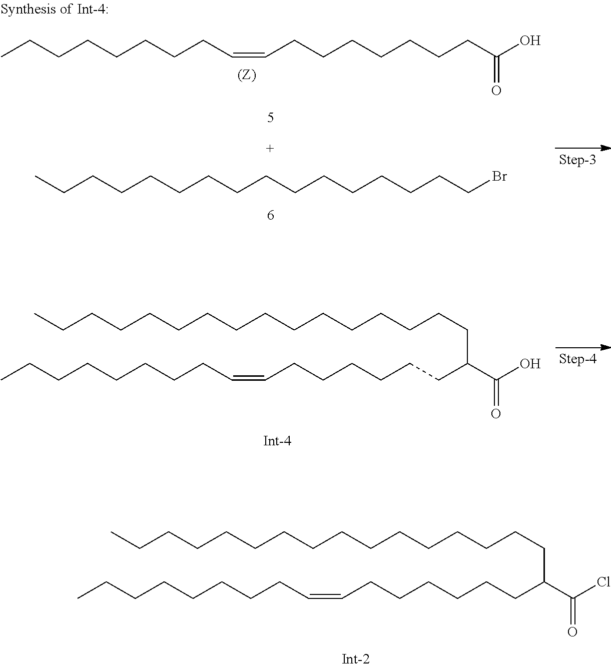

### Synthetic Procedure

### Step-1: Synthesis of Int-3

Choline glycerophosphate (2 g, 0.0077 mol) and dibutyltin oxide (DBTO) (2.12 g, 0.00854 mol) were suspended in 2-propanol (100 mL) and refluxed for 18 h at 90° C. The mixture was cooled to 0 to 5° C., TEA (1.3 mL, 0.00932 mol) and Int-2 (4.89 g, 0.00932 mol) were added. Resulting reaction mixture was stirred over a period of 36 h at 25-30° C. under argon atmosphere. Progress of the reaction was monitored by TLC. TLC shows new polar spot formation.

TLC eluent: (20% MeOH in DCM and 5% Aq NH₃)-KMnO₄ and Molybdenum blue active

Work up: Reaction mass was diluted with purified water (250 mL) and extracted with heptane (250 mL). The water-alcohol solution was extracted three times with heptane (3×250 mL) and resulting aqueous layer evaporated under reduced pressure at 45° C. to dryness. Obtained crude was purified by flash column (10% MeOH/DCM & 1% H₂O) to get Int-3 as an off-white sticky solid. Desired product was confirmed by ¹H NMR.

Yield: 2 g (34.54%); Characterized by ¹H-NMR

### Step-2:—Synthesis of Lipid-13

To a solution of Int-3 (1.5 g, 0.00201 mol) in DCM (75 mL, 50 V) at 0 to 5° C., were added Int-4 (1.22 g, 0.00241 mol), 2-methyl-6-nitrobenzoic anhydride [MNBA](3.52 g, 0.0102 mol) and DMAP (2.45 g, 0.0102 mol). Resulting reaction mixture was stirred for 48 h at 25-30° C. Progress of the reaction was monitored by TLC. TLC shows new nonpolar spot formation.

TLC eluent: (15% MeOH in DCM and 5% Aq NH₃) Molybdenum blue & KMnO₄ active

Work up: Reaction mixture was directly concentrated under reduced pressure at 40° C. Residue was dissolved in DCM and was purified flash column using neutral alumina with eluents (10% MeOH/DCM & 1% H₂O) to afford Lipid-13 as pale blue semi-solid.

Yield: 420 mg (16.9%); Characterized by ¹H-NMR and Q-TOF

Q-TOF: (EI, m/z) calcd for C₇₆H₁₄₈O₈P [M]⁺: 1235.1; HPLC (CAD) Purity: 74.7%

¹H NMR (400 MHz, CD₃OD): 5.38-5.31 (4H, m), 4.63-4.6 (1H, m), 4.29 (2H, m), 4.0-4.11 (1H, m), 3.99-3.89 (2H, m), 3.72-3.63 (2H, m), 3.23 (9H, s), 2.36-2.31 (2H, m), 2.04-2.03 (8H, m), 1.63-1.60 (5H, m), 1.47-1.45 (5H, m), 1.37-1.26 (98H, m), 0.94-0.88 (9H, m);

### Step-3: Synthesis of 2-Hexadecyl-octadec-9-enoic Acid (Int-4)

In a 3 neck RB THF (50 mL, 10 V) was cooled to −5 to 0° C., added LDA-2M in THF (31.7 mL, 0.0635 mol) in a dropwise manner by maintaining temperature −5 to 0° C., after completion of addition added pre dissolved solution of Int-5 (10 g, 0.0353 mol) dissolved in THF (50 mL, 5V) dropwise, followed by HMPA (6.93 mL, 0.0398 mol). RM was immediately brought to ambient temperature and stirred for 30 minutes. RM was once again cooled to 0 to −5° C. and added Int-6 (10.8 g, 0.0353 mol) in a single addition. RM was immediately brought to ambient temperature and stirred for 16 h at RT. RM was monitored through TLC. TLC shows nonpolar spot formation along with SM.

TLC eluent: 20% in EtOAc in Hexane

Work up:—Reaction mass was cooled to −5-0° C., quenched with the addition of cold 1.5 N HCl (500 mL) by maintaining −5 to 0° C. The resultant reaction mixture was warmed to room temperature and extracted with ethyl acetate (500 mL×3). Organic layers were dried over anhydrous sodium sulphate and concentrated under reduced pressure at 45° C. to afford crude. Crude was purified through combiflash, product eluted at 2-3% ethyl acetate-hexane to afford Int-4 as off-white sticky solid.

Yield: 11.0 g (61.4%); Characterized by ¹H-NMR

### Step-4: Synthesis of 2-Hexadecyl-octadec-9-enoyl Chloride (Int-2)

A stirred solution of Int-4 (4.8 g, 0.00946 mol) in DCM (48 mL, 10 V) was cooled to at 0-10° C., Oxalyl chloride (2.62 mL, 0.0189 mol) was added in a dropwise manner at 0-5° C. followed by DMF (0.192 mL, 0.04 V). Reaction mixture was stirred at 25-30° C. for 5 h. Progress of reaction monitored by TLC by quenching in methanol. TLC shows new non-polar spot formation.

TLC eluent: 10% EtOAc in Hexane (KMnO4 active)

Work up: Reaction mixture was evaporated to dryness under reduced pressure and as such taken for next step.

Yield: 5.0 g, (Crude)

### Example 14: Synthesis of Lipid-14

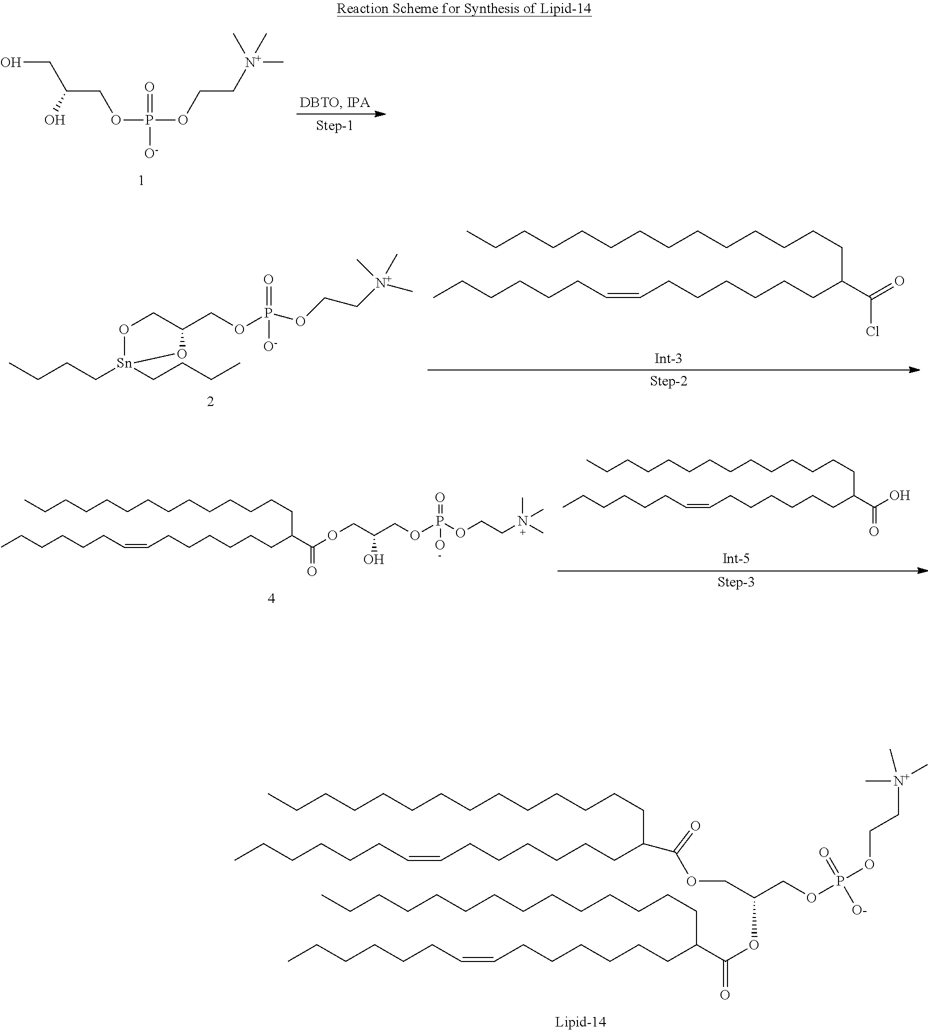

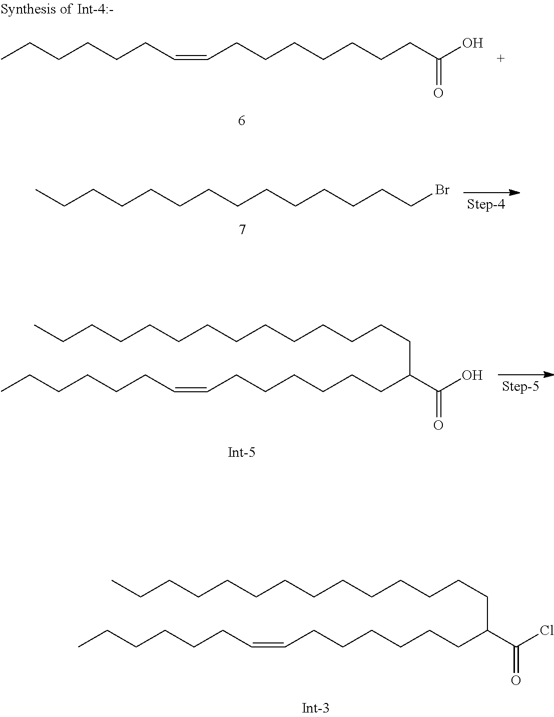 [SMILES: CCCCCC/C=C\CCCCCCCC(=O)O ; CCCCCCCCCCCCCCBr ; CCCCCC/C=C\CCCCCCC(CCCCCCCCCCCCCC)C(=O)O ; CCCCCC/C=C\CCCCCCC(CCCCCCCCCCCCCC)C(=O)Cl]

### Synthetic Procedure

### Step-1&2: Synthesis of Int-4

Choline glycerophosphate (2 g, 0.0077 mol) and dibutyltin oxide (DBTO) (2.12 g, 0.0085 mol) were suspended in 2-propanol (100 mL) and refluxed at 90° C. for 18 h. The mixture was cooled to 0° C. to 5° C., TEA (1.3 mL, 0.0093 mol) and Int-3 (4.37 g, 0.0093 mol) were added. Resulting reaction mixture was stirred over a period of 18 h at 25-30° C. under argon atmosphere. Progress of the reaction was monitored by TLC. After completion of the reaction, reaction mass was filtered through celite bed and bed was washed with 2-propanol (100 mL). Filtrate was collected and diluted with purified water (250 mL). The water-alcohol solution was extracted three times with heptane (3×250 mL) and resulting organic layer evaporated under reduced pressure at 45° C. to dryness to get crude. Crude was purified through neutral alumina column using CH₂Cl₂:MeOH:H₂O (65/25/4) to afford Int-4 as pale green waxy solid.

Yield: 2.0 g, (37.7%); Characterized by ¹H-NMR

### Step-3: Synthesis of Lipid-14

To a stirred solution of Int-4 (2.0 g, 0.0028 mol) in DCM (100 mL, 50 V) was added Int-5 (1.95 g, 0.0043 mol) and 2-methyl-6-nitrobenzoic anhydride [MNBA](5.08 g, 0.0147 mol) and DMAP (3.54 g, 0.0289 mol) at 0-5° C. Resulting reaction mixture was stirred for 48 h at 25-30° C. Progress of the reaction was monitored by TLC. After completion of the reaction, reaction mixture was directly concentrated under reduced pressure at 40° C. Residue was treated with hexane (100 mL) and filtered off. Filtrate was evaporated at 45° C. to get crude and it was purified through neutral alumina column using CH₂Cl₂:MeOH:H₂O (65/25/4) to obtain partially pure material. Which was dissolved in DCM and added water (solid appears) then DCM was evaporated, the solid was filtered, washed with water and dried under reduced pressure at 40° C. to afford Lipid-14 as pale-yellow waxy solid.

Yield: (0.765 g, 31%); Characterized by ¹H-NMR and Q-TOF LCMS (Q-TOF): (EI, m/z) calcd for C₆₈H₁₃₂NO₈P [M+H]: 1122.96; HPLC (CAD) purity: 85.36% ¹H NMR (400 MHz, MeOD): 5.49-5.31 (4H, m), 5.26-5.24 (1H, m), 4.62-4.59 (1H, m), 4.29 (2H, m), 4.12-4.10 (1H, m), 4.082-3.95 (2H, m), 3.66-3.63 (2H, s), 3.23 (9H, S), 2.36-2.32 (2H, m), 2.04-2.03 (8H, m), 1.61-1.60 (4H, m), 1.47 (4H, m), 1.29 (80H, m), 0.91 (12H, t, J=4.4 Hz)

### Step-4: Synthesis of Int-5

To A RBF containing THF (100 mL, 5.5 V) was added LDA 2M in THF (88.4 mL, 0.1169 mol), pre-dissolved solution of Int-6 (18.0 g, 0.0707 mol) in THF (80 mL) followed by HMPA (13.91 mL, 0.079 mol) were added slowly at 0° C. Reaction mixture was allowed to attain 25-30° C. and stirred at same temperature for 30 min. After 30 min, RM was cooled to 0° C. and added Int-7 (21.57 g, 0.077 mol) rapidly. RM was allowed to attain 25-30° C. and stirred for 2 h at same temperature. Progress of the reaction was monitored by TLC. After the completion of reaction, reaction mixture was quenched by adding 1.5 M HCl (300 ml) at 0-5° C. and extracted with Ethyl acetate (2×300 ml). Combined organic layer was dried over anhydrous Na2SO4 and concentrated to get the crude product which was purified by Combiflash using 0-2% of Ethyl acetate in Hexanes to afford Int-5 as off-white solid.

Yield: 10.5 g (29.2%); Characterized by ¹H-NMR

### Step-5: Synthesis of Int-3

A stirred solution of Int-4 (4.2 g, 0.0094 mol) in DCM (42 mL, 10 V) was cooled to 0-10° C., Oxalyl chloride (1.59 mL, 0.0186 mol) was added in a drop wise manner at 0-5° C. and catalytic DMF (0.1 mL) was added. Resulting reaction was stirred for 3 h at room temperature. Progress of the reaction was monitored by TLC. After the completion of reaction, reaction mixture was concentrated under reduced pressure at 40° C. to dryness to afford Int-3 as yellow liquid. Crude was taken for next step immediately.

Yield: 5 g (Crude)

### Example 15: Synthesis of Lipid-15

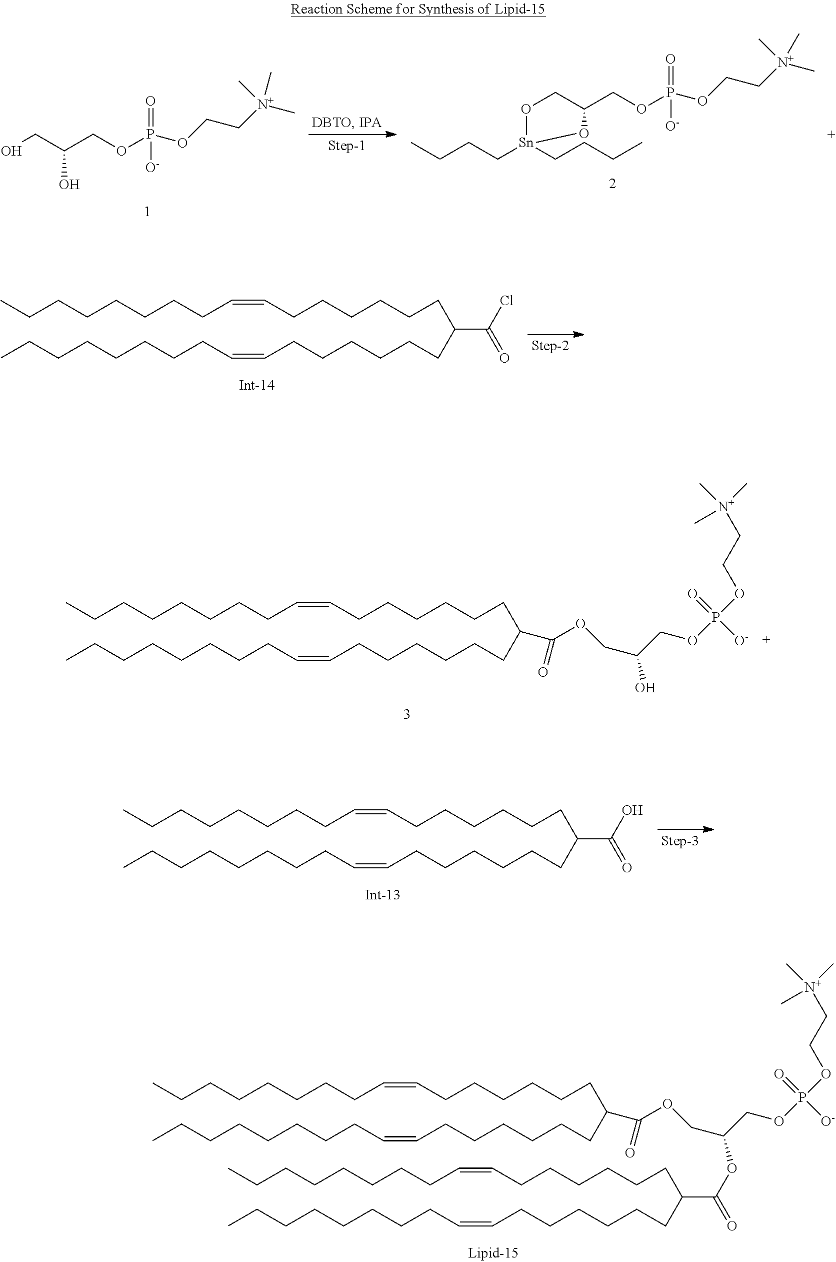

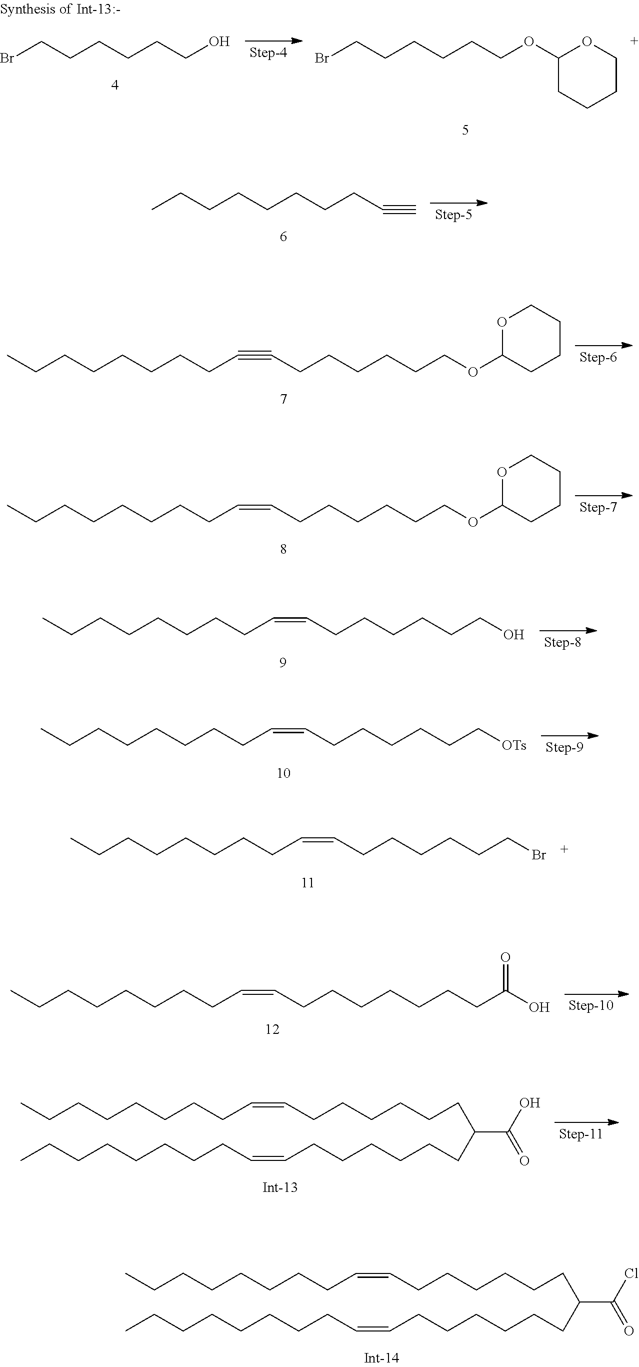

### Synthetic Procedure

### Step-1&2: Synthesis of Int-3

Step-1: To a stirred solution of Int-1 (2 g, 0.0077 mol) in 2-propanol (100 mL, 50 V) was added Dibutyltin oxide (2.12 g, 0.0085 mol) at 25-30° C. Resulting suspension was stirred at 90° C. for 18 h.
Step-2: Above reaction mixture was cooled to 0° C. and added triethylamine (1.32 mL, 0.00077 mol) followed by pre-dissolved solution of Int-14 (4.87 g, 0.0093 mol) in DCM (6 mL). Resulting suspension was allowed to attain 25-30° C. and stirred at same temperature for 48 h.

Progress of the reaction was monitored by TLC. After the completion of reaction, reaction mixture was evaporated completely to get crude. Crude was purified by flash column using neutral alumina, desired product was eluted at 8:2:1 (DCM:MeOH:Water). Pure fractions were evaporated to afford Int-3 as waxy solid.

Yield: 2.4 g (42.6%); Characterized by ¹H-NMR

### Step-3: Synthesis of Lipid-15

To a stirred solution of Int-3 (2.1 g, 0.0028 mol) and Int-13 (1.7 g, 0.0033 mol) b in DCM (105 mL, 50 V) were added 2-methyl-6-nitrobenzoic anhydride (MNBA) (4.95 g, 0.014 mol) and DMAP (3.45 g, 0.028 mol) at 0-5° C. RM was allowed to attain 25-30° C. and stirred for 2 h at same temperature for 48 h. Progress of the reaction was monitored by TLC. After completion of reaction, reaction mixture was evaporated completely to get crude. Crude was purified by flash column using neutral alumina to obtain partially purified BMS-L206 which was further purified in Combi flash using 8:2:1 (DCM:MeOH:water) to afford Lipid-15 as white waxy solid.

Yield: 0.65 g (18.7%); Characterized by ¹H-NMR and Q-TOF LCMS (Q-TOF): (EI, m/z) calcd for C₇₆H₁₄₄NO₈P [M+H]: 1231.05; HPLC (CAD) Purity: 62.80% ¹H NMR (400 MHz, MeOD): 5.38-5.35 (8H, m), 5.26-5.24 (1H, m), 4.62-4.59 (1H, m), 4.28 (2H, m), 4.11-4.08 (1H, m), 4.06-3.98 (2H, m), 3.64 (2H, m), 3.21 (9H, s), 2.36-2.34 (2H, m), 1.99 (16H, m), 1.60 (4H, m), 1.47 (4H, m), 1.29 (80H, m), 0.89 (12H, t, J=6.4 Hz)

### Step-4: Synthesis of 2-(6-Bromo-hexyloxy)-tetrahydro-pyran (Int-5)

To a stirred solution of Int-4 (35 g, 0.193 mol) in DCM (350 ml, 10 V) was added 3,4 dihydro-2H pyran (51.14 ml, 0.56 mol) at 0-5° C. followed by PTSA monohydrate (367 mg, 0.0019 mol) in two equal lots. Resulting reaction mixture was stirred at 25-30° C. for 5 h. Progress of reaction was monitored by TLC. After the completion of reaction, reaction mixture was quenched by adding 10% NaHCO₃ solution (200 ml) at 0-5° C. and extracted with DCM (2×350 ml). Combined organic layer was dried over anhydrous Na₂SO₄ and concentrated to get crude, which was purified by Combiflash using 0-2.5% of Ethyl acetate in Hexanes to afford 2-(6-Bromo-hexyloxy)-Tetrahydro-pyran (Int-5) as colour less thick liquid.

Yield: 30.5 g (59.5%); Characterized by ¹H-NMR

### Step-5: Synthesis of 2-Hexadec-7-ynyloxy-tetrahydro-pyran (Int-7)

To a stirred solution of Int-6 (30 g, 0.169 mol) THF (300 ml, 10 V), were added HMPA (29.5 ml, 0.169 mol) and n-BuLi 2M in THF (90.4 ml, 0.169 mol) in a dropwise manner at −25 to −30° C. Resulting brown coloured reaction mass was stirred at −30° C. for 30 min. After 30 min, Int-5 (30.5 ml, 0.113 mol) was added dropwise at −25 to −30° C. Resulting reaction mixture was allowed to attain 25-30° C. and stirred at same temperature for 16 h. Progress of the reaction was monitored by TLC. After completion of reaction, the reaction mixture was quenched by adding 1.5 M HCl (200 ml) at 0-5° C. and extracted with Ethyl acetate (2×300 ml). Combined organic layer was dried over anhydrous Na₂SO₄ and concentrated under reduced pressure to get the crude product which was purified by Combiflash using 3-4% of Ethyl acetate in Hexanes to afford 2-Hexadec-7-ynyloxy-tetrahydro-pyran (Int-7) as pale-yellow thick liquid.

Yield: 14.2 g (39%); Characterized by ¹H-NMR

### Step-6: Synthesis of 2-Hexadec-7-ynyloxy-tetrahydro-pyran (Int-8)

To a stirred solution of Int-7 (14.4 g, 0.044 mol) in Ethyl acetate (576 mL, 40 V), was degassed with argon for 10 min and added Lindlar catalyst (720 mg, 5% w/w) and stirred under H₂ gas for 18 h at 25-30° C. Progress of the reaction was monitored by TLC. After 18 h, TLC shows formation of new spot with complete consumption of Int-7. The reaction mixture was filtered through celite bed and bed was washed with ethyl acetate (500 mL). Filtrate was concentrated to get 2-Hexadec-7-ynyloxy-tetrahydro-pyran (Int-8) as pale-yellow thick liquid.

Yield: 14.1 g (crude); Characterized by ¹H-NMR

### Step-7: Synthesis of 2 Hexadec-7-en-1-ol (Int-9)

To a stirred solution of Int-8 (14 g, 0.043) in Methanol was added PTSA monohydrate (6.56 g, 0.034 mol) in two equal lots at 0-5° C. Resulting reaction mixture was allowed to stir at 25-30° C. for 3 h. Progress of the reaction was monitored by TLC. After completion of reaction, the reaction mixture was quenched by adding solid NaHCO₃ (20 g) at 10-15° C. and filtered off. Filtrate was evaporated to get white residue which was treated with DCM (50 ml) and filtered off. Filtrate was evaporated to obtain Hexadec-7-en-1-ol (Int-9) as thick liquid.

Yield: 9.1 g (86%); Characterized by ¹H-NMR

### Step-8: Synthesis of Toluene-4-sulfonic acid hexadec-7-enyl ester (Int-10)

To a stirred solution of Int-9 (9 g, 0.037 mol) in DCM (90 mL, 10 V) was added TEA (18.26 mL, 0.131 mol) resulting solution was allowed to stir at 0-5° C. for 15 min and added Tosyl chloride (21.42 g, 0.131 mol) at same temperature. Resulting reaction mixture was allowed to stir at 25-30° C. for 24 h. Progress of the reaction was monitored by TLC. After completion of reaction, the reaction mixture was filtered off. Filtrate was evaporated to get crude which was purified by Combi-flash using 5-7% of Ethyl acetate in Hexanes to afford Toluene-4-sulfonic acid hexadec-7-enyl ester (Int-10) as pale-yellow liquid.

Yield: 11.1 g (75%); Characterized by ¹H-NMR

### Step-9: Synthesis of 1-Bromo-hexadec-7-ene (Int-11)

To a stirred solution of Int-10 (11 g, 0.027 mol) in THF (110 ml, 10 V) was added Magnesium bromide ethyl etherate (20.89 g, 0.080 mol) at 25-30° C. Resulting suspension was stirred at 40° C. for 18 h. After 18 h, TLC shows the complete consumption of Int-10. To the reaction mixture, water was added and extracted with ethyl acetate (100 mL×2). Combined organic layer was dried over anhydrous Na₂SO₄ and concentrated to get the crude which was purified by Combiflash using 0-1% of Ethyl acetate in Hexanes to afford 1-Bromo-hexadec-7-ene (Int-11) as pale colour less thick liquid.

Yield: 7.7 g (87%); Characterized by ¹H-NMR

### Step-10: Synthesis of 2-Hexadec-7-enyl-octadec-9-enoic Acid (Int-13)

To A RB flask containing THF (70 mL) was added LDA 2M in THF (28 mL, 0.055 mol), pre-dissolved solution of Int-12 (7.9 g, 0.027 mol) in THF (10 mL) followed by HMPA (5.5 mL, 0.031 mol) were added slowly at 0° C. RM was allowed to attain 25-30° C. and stirred at same temperature 30 min. After 30 min, RM was cooled to 0° C. and added Int-11 (9.33 g, 0.030 mol) rapidly. RM was allowed to attain 25-30° C. and stirred for 2 h at same temperature. Progress of the reaction was monitored by TLC. After completion of reaction, reaction mixture was quenched by adding 1.5 M HCl (50 ml) at 0-5° C. and extracted with Ethyl acetate (2×300 ml). Combined organic layer was dried over anhydrous Na₂SO₄ and concentrated to get the crude product, which was purified by Combiflash using 3-4% of Ethyl acetate in Hexanes to afford Hexadec-7-enyl-octadec-9-enoic acid (Int-13) as pale-yellow thick liquid.

Yield: 10 g (70.8%); Characterized by 4H-NMR

### Step-11: Synthesis of 2-Hexadec-7-enyl-octadec-9-enoyl Chloride (Int-14)

To a stirred solution of Int-13 (4.8 g, 0.0095 mol) in DCM (48 mL, 10 V) was added Oxalyl chloride (2.39 g, 0.019) followed by catalytic DMF (0.1 mL) at 0-5° C. Reaction mixture was allowed to attain 25-30° C. and stirred at same temperature for 5 h. Progress of the reaction was monitored by TLC. After the completion of reaction, reaction mixture was evaporated completely to obtain Int-14 (4.8 g) as pale-yellow thick liquid. Crude was taken as such for next reaction.

TABLE 1

| Lipid | Structure |
|---|---|
| Lipid-1 | 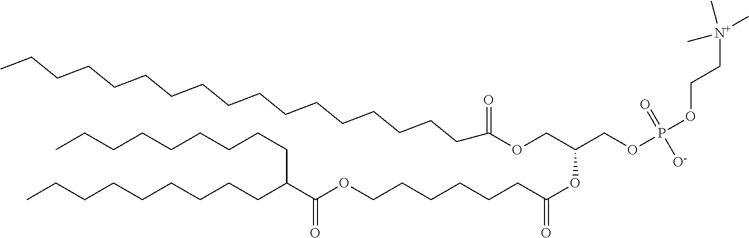 [SMILES: CCCCCCCCCCCCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-2 | 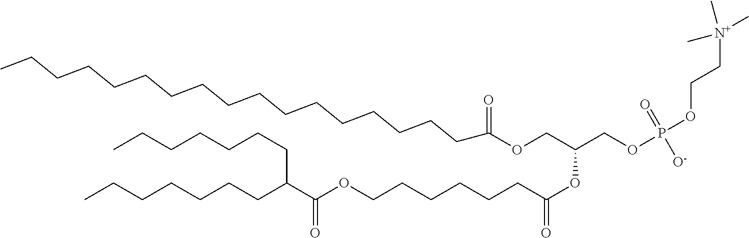 [SMILES: CCCCCCCCCCCCCCCCCC(=O)OC[C@H](COP(=O)([O-])OCC[N+](C)(C)C)OC(=O)CCCCCCOC(=O)C(CCCCCCC)CCCCCCC] |
| Lipid-3 | 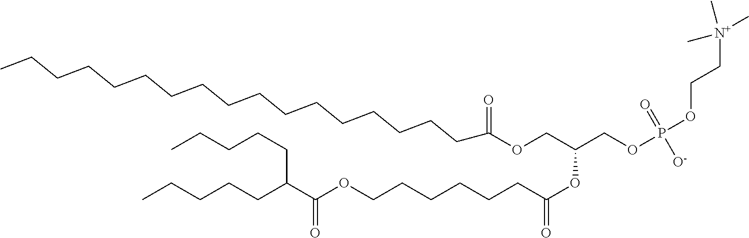 [SMILES: CCCCCCCCCCCCCCCCCC(=O)OC[C@H](COP(=O)([O-])OCC[N+](C)(C)C)OC(=O)CCCCCCOC(=O)C(CCCCC)CCCCC] |
| Lipid-4 | 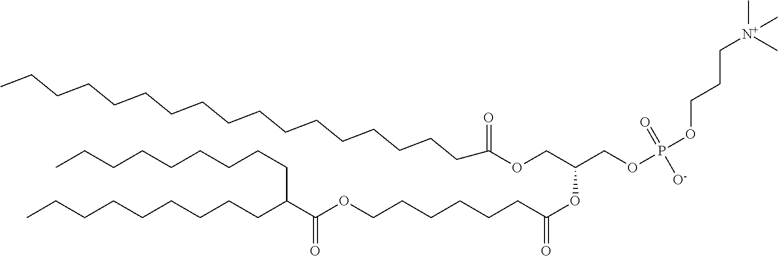 [SMILES: CCCCCCCCCCCCCCCCCC(=O)OC[C@H](COP(=O)([O-])OCCC[N+](C)(C)C)OC(=O)CCCCCCOC(=O)C(CCCCCCCCC)CCCCCCCCC] |
| Lipid-5 | 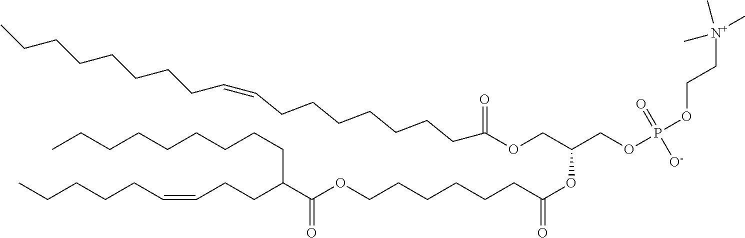 [SMILES: CCCCC/C=C\CCC(CCCCCCCCC)C(=O)OCCCCCCC(=O)O[C@H](COC(=O)CCCCCCC/C=C\CCCCCCCC)COP(=O)([O-])OCC[N+](C)(C)C] |
| Lipid-6 | 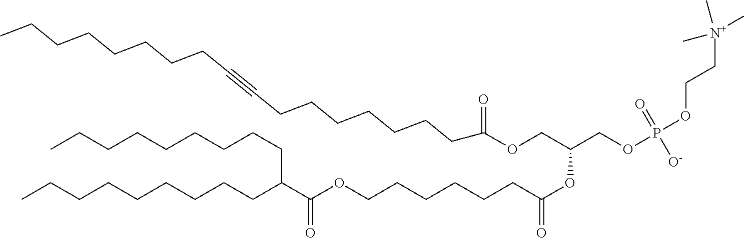 [SMILES: CCCCCCCCC#CCCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-7 | 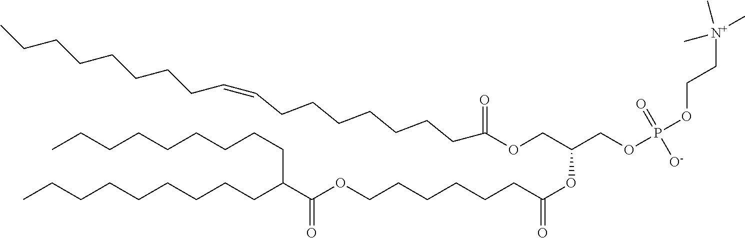 [SMILES: CCCCCCCC/C=C\CCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-8 | 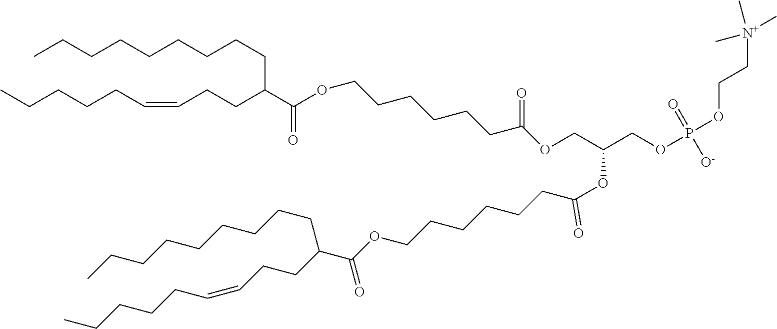 [SMILES: CCCCC/C=C\CCC(CCCCCCCCC)C(=O)OCCCCCCC(=O)OC[C@H](COP(=O)([O-])OCC[N+](C)(C)C)OC(=O)CCCCCCOC(=O)C(CC/C=C\CCCCC)CCCCCCCCC] |
| Lipid-9 | 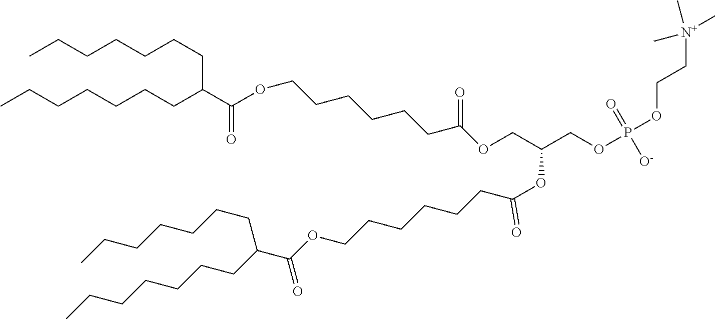 [SMILES: CCCCCCCC(CCCCCCC)C(=O)OCCCCCCC(=O)OC[C@H](COP(=O)([O-])OCC[N+](C)(C)C)OC(=O)CCCCCCOC(=O)C(CCCCCCC)CCCCCCC] |
| Lipid-10 | 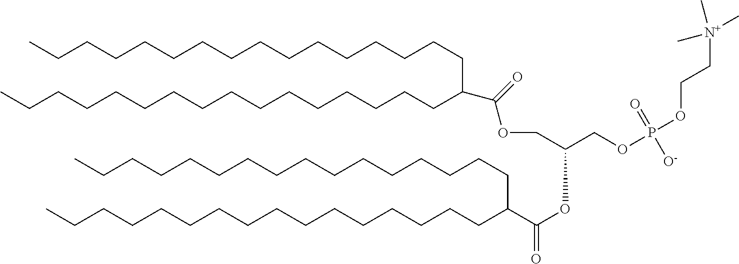 [SMILES: CCCCCCCCCCCCCCCCC(CCCCCCCCCCCCCCCC)C(=O)OC[C@H](COP(=O)([O-])OCC[N+](C)(C)C)OC(=O)C(CCCCCCCCCCCCCCCC)CCCCCCCCCCCCCCCC] |
| Lipid-11 | 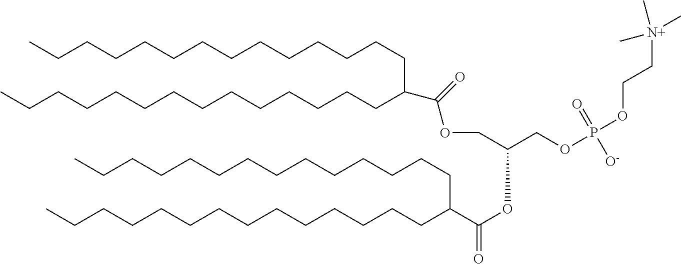 [SMILES: CCCCCCCCCCCCCCC(CCCCCCCCCCCCCC)C(=O)OC[C@H](COP(=O)([O-])OCC[N+](C)(C)C)OC(=O)C(CCCCCCCCCCCCCC)CCCCCCCCCCCCCC] |
| Lipid-12 | 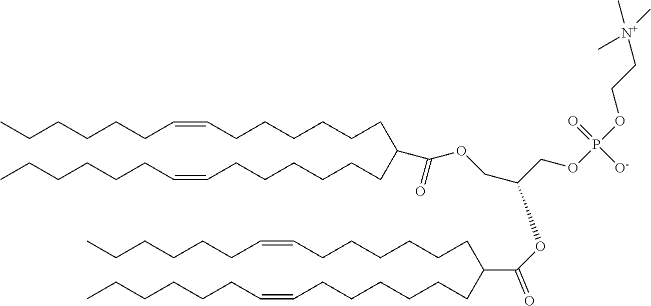 [SMILES: CCCCCC/C=C\CCCCCCC(CCCCCC/C=C\CCCCCC)C(=O)OC[C@H](COP(=O)([O-])OCC[N+](C)(C)C)OC(=O)C(CCCCCC/C=C\CCCCCC)CCCCCC/C=C\CCCCCC] |
| Lipid-13 | 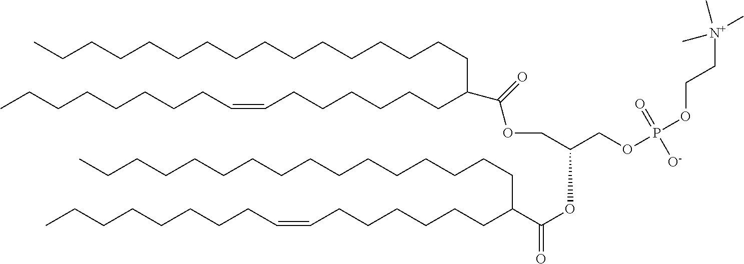 [SMILES: CCCCCCCC/C=C\CCCCCCC(CCCCCCCCCCCCCCCC)C(=O)OC[C@H](COP(=O)([O-])OCC[N+](C)(C)C)OC(=O)C(CCCCCC/C=C\CCCCCCCC)CCCCCCCCCCCCCCCC] |
| Lipid-14 | 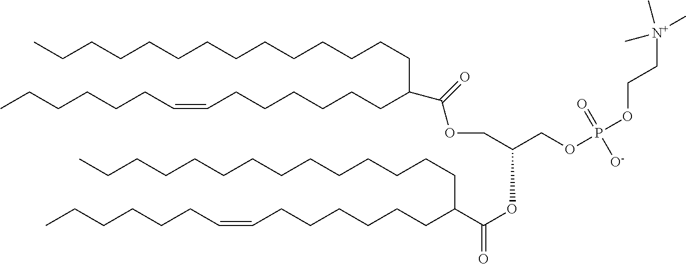 [SMILES: CCCCCC/C=C\CCCCCCC(CCCCCCCCCCCCCC)C(=O)OC[C@H](COP(=O)([O-])OCC[N+](C)(C)C)OC(=O)C(CCCCCC/C=C\CCCCCC)CCCCCCCCCCCCCC] |
| Lipid-15 | 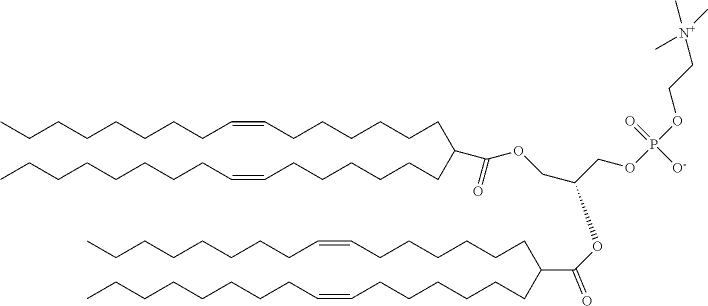 [SMILES: CCCCCCCC/C=C\CCCCCCC(CCCCCC/C=C\CCCCCCCC)C(=O)OC[C@H](COP(=O)([O-])OCC[N+](C)(C)C)OC(=O)C(CCCCCC/C=C\CCCCCCCC)CCCCCC/C=C\CCCCCCCC] |

Using 10000 ethanol, stock solutions (20 mg/mL) of cholesterol, Pegylated lipid, DSPC, D-Lin-MC3 or another ionizable lipid, and branched phospholipid Lipid (1-15) were made. The resulting lipid solutions were then combined to yield a molar ratio of 50% ionizable lipid, 0-10% DSPC, 0-10% branched phospholipid, and 39% cholesterol. An aqueous solution (0.214 mg/mL) of eGFP mRNA (5 moU modified, Trilink) was made using 50 mM pH 3.5 citrate buffer and mixed using a Benchtop NanoAssembr (Cytiva) at a 2:1 flow rate ratio at a Nitrogen:Phosphorus (N:P) ratio of 9. Immediately after mixing, the formulation was diluted 50% by volume with 50 mM pH 3.5 citrate buffer and dialyzed against an excess of DPBS overnight in a cold room (4 C). After overnight dialysis, particle size and PDI were measured using a Malvern Zeta-sizer and mRNA % Encapsulation efficiency was quantified using a standard Ribogreen Assay.

| Reagent | Origin | Decription |
|---|---|---|
| Lipid of | | |
| Formula I | | |
| D-Lin-MC3- | MedChemExpress | D-Lin-MC3-DMA is an |
| DMA | | ionizable cationic lipid |
| Citric acid | Sigma | |
| DPBS | Cytiva | DPBS/Modified (—Ca, —Mg) |
| DMG-PEG | Avanti | 1,2-dimyristoyl-rac-glycero- |
| | | 3-methoxypolyethylene |
| | | glycol-2000, this product |
| | | is a mixture of 1,2-DMG |
| | | PEG2000 and 1,3-DMG PEG2000 |
| | | in ~97:3 ratio. |
| DSPC | Avanti | 18:0 PC (DSPC) |
| | | 1,2-distearoyl-sn- |
| | | glycero-3-phosphocholine |
| Cholesterol | Sigma | powder, BioReagent, suitable |
| | | for cell culture, ≥99% |
| Ethanol | Sigma | |
| eGFP mRNA | Trilink | 5-methoxyuridine modified |
| (5moU) | | mRNA designed to express |
| | | green fluorescent protein |

We claim:

1. A compound having the structure of Formula I: 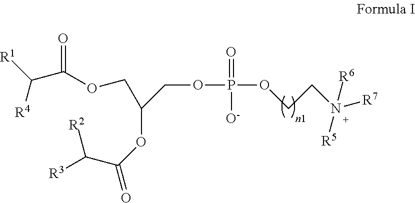
or a pharmaceutically acceptable salt thereof,
Wherein n1 is an integer selected from 0, 1 or 2;
Each R¹, R², R³ and R⁴ is independently selected from C₄₋₂₀ alkyl and C₄₋₂₀ alkenyl;
wherein said R¹, R², R³ and R⁴ is optionally substituted with one or two —O(C(O))—C₄₋₁₆ alkyl, —O(C(O))—C₄₋₁₆ alkenyl or —O(C(O))—C₄₋₁₆ alkynyl;
Each R⁵, R⁶ and R⁷ is independently selected from H and C₁₋₃ alkyl.

2. A compound of claim 1 having the structure of Formula II: 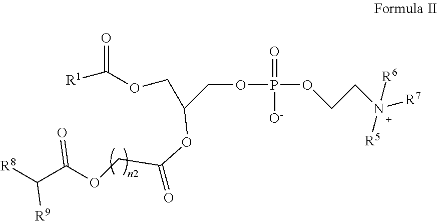
or a pharmaceutically acceptable salt thereof;
wherein, each R⁸ and R⁹ is independently selected from C₄₋₁₀ alkyl and C₄₋₁₀ alkenyl; and,
n2 is an integer selected from 4, 5, 6, 7 or 8.

3. A compound of claim 1 having the structure of Formula III: 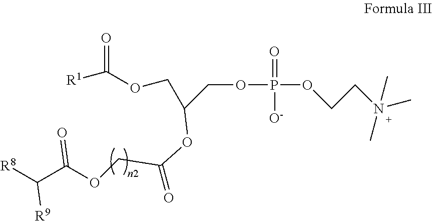
or a pharmaceutically acceptable salt thereof.

4. A compound of claim 1 having the structure of Formula IV: 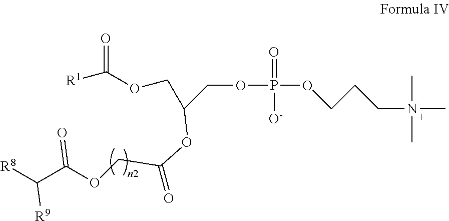
or a pharmaceutically acceptable salt thereof.

5. A compound of claim 1 having the structure of Formula V: 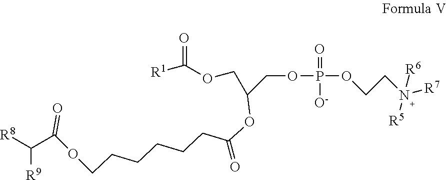
or a pharmaceutically acceptable salt thereof.

6. A compound of claim 1 having the structure of Formula VI: 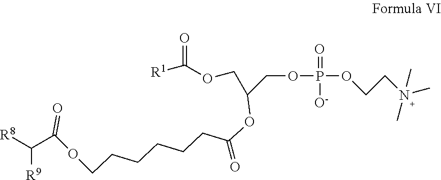
or a pharmaceutically acceptable salt thereof.

7. A compound according to claim 1, wherein R¹ is selected from C₁₆₋₂₀ alkyl, C₁₆₋₂₀ alkenyl, preferably C₁₇ alkyl or C₁₇ alkenyl.

8. A compound according to claim 1 wherein R² is selected from C₆₋₁₂ alkyl and C₆₋₁₂ alkenyl, preferably C₉ alkenyl and C₉ alkyl.

9. A compound according to claim 1 wherein each R³ is selected from: C₆₋₁₂ alkyl and C₆₋₁₂ alkenyl, preferably C₉ alkenyl and C₉ alkyl.

10. A compound according to claim 1, wherein each R¹, R², R³ and R⁴ is independently selected from:

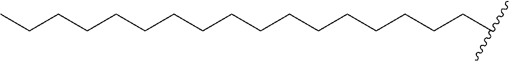 [SMILES: *CCCCCCCCCCCCCCCCC]

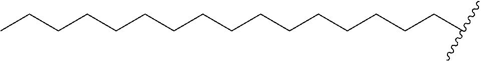 [SMILES: *CCCCCCCCCCCCCCCC]

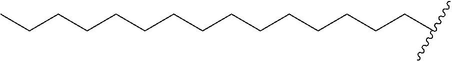 [SMILES: *CCCCCCCCCCCCCCC]

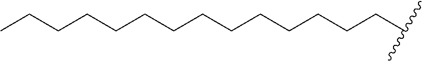 [SMILES: *CCCCCCCCCCCCCC]

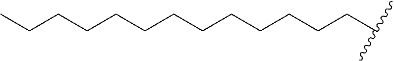 [SMILES: *CCCCCCCCCCCCC]

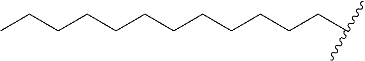 [SMILES: *CCCCCCCCCCCC]

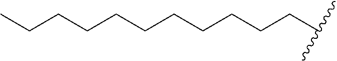 [SMILES: *CCCCCCCCCCC]

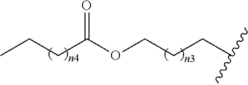

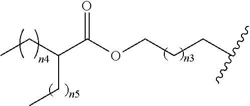

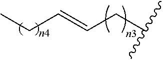

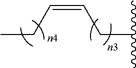

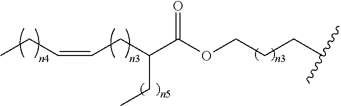

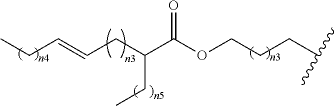

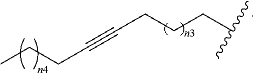

11. A compound of claim 1 selected from Table 1:
TABLE 1

| Lipid | Structure |
|---|---|
| Lipid-1 | 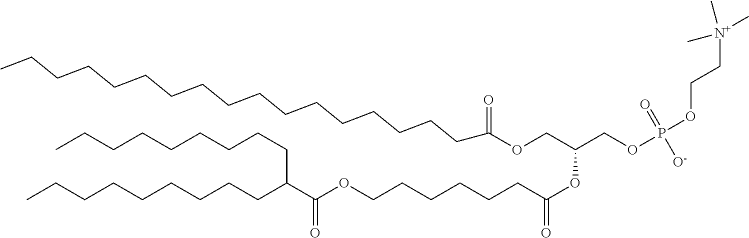 [SMILES: CCCCCCCCCCCCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-2 | 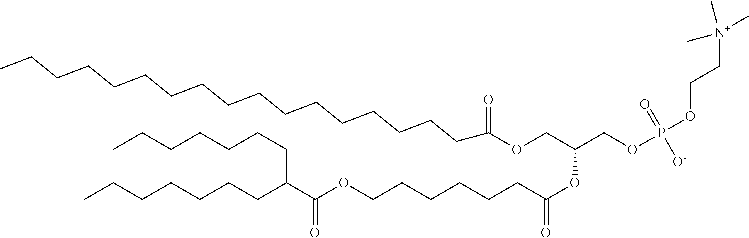 [SMILES: CCCCCCCCCCCCCCCCCC(=O)OC[C@H](COP(=O)([O-])OCC[N+](C)(C)C)OC(=O)CCCCCCOC(=O)C(CCCCCCC)CCCCCCC] |
| Lipid-3 | 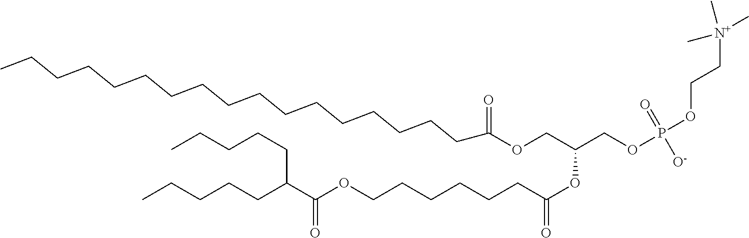 [SMILES: CCCCCCCCCCCCCCCCCC(=O)OC[C@H](COP(=O)([O-])OCC[N+](C)(C)C)OC(=O)CCCCCCOC(=O)C(CCCCC)CCCCC] |
| Lipid-4 | 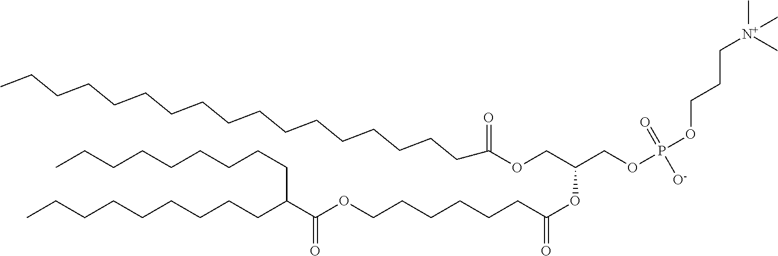 [SMILES: CCCCCCCCCCCCCCCCCC(=O)OC[C@H](COP(=O)([O-])OCCC[N+](C)(C)C)OC(=O)CCCCCCOC(=O)C(CCCCCCCCC)CCCCCCCCC] |
| Lipid-5 | 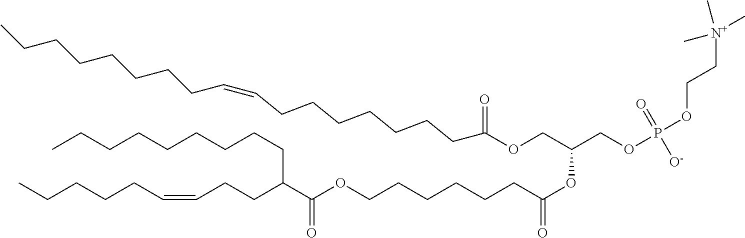 [SMILES: CCCCC/C=C\CCC(CCCCCCCCC)C(=O)OCCCCCCC(=O)O[C@H](COC(=O)CCCCCCC/C=C\CCCCCCCC)COP(=O)([O-])OCC[N+](C)(C)C] |
| Lipid-6 | 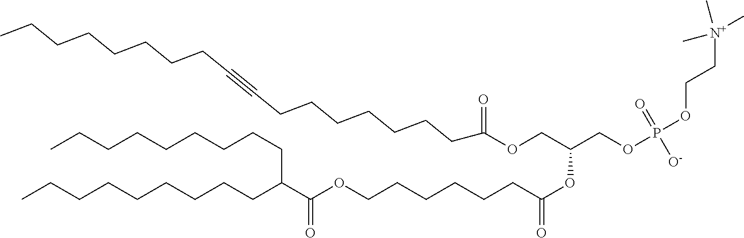 [SMILES: CCCCCCCCC#CCCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-7 | 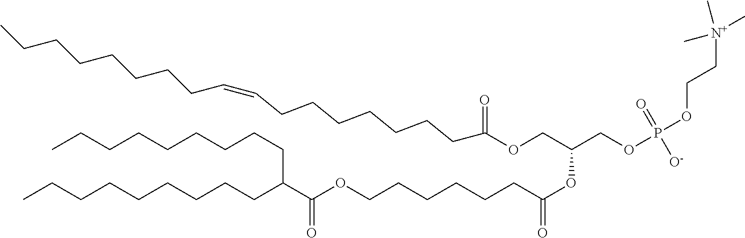 [SMILES: CCCCCCCC/C=C\CCCCCCCC(=O)OC[C@H](COP(=O)([O-])OCC[N+](C)(C)C)OC(=O)CCCCCCOC(=O)C(CCCCCCCCC)CCCCCCCCC] |
| Lipid-8 | 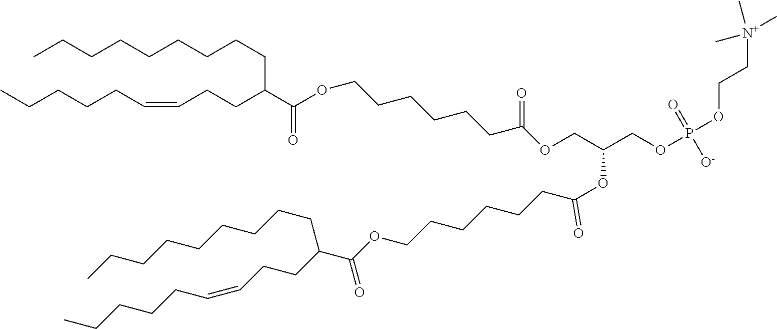 [SMILES: CCCCC/C=C\CCC(CCCCCCCCC)C(=O)OCCCCCCC(=O)OC[C@H](COP(=O)([O-])OCC[N+](C)(C)C)OC(=O)CCCCCCOC(=O)C(CC/C=C\CCCCC)CCCCCCCCC] |
| Lipid-9 | 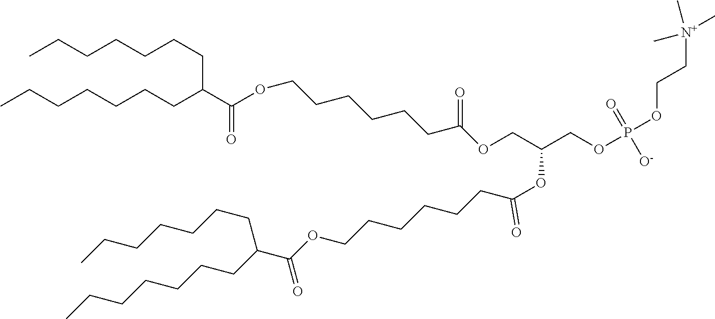 [SMILES: CCCCCCCC(CCCCCCC)C(=O)OCCCCCCC(=O)OC[C@H](COP(=O)([O-])OCC[N+](C)(C)C)OC(=O)CCCCCCOC(=O)C(CCCCCCC)CCCCCCC] |
| Lipid-10 | 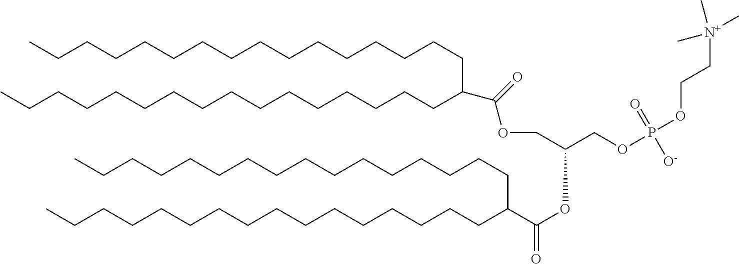 [SMILES: CCCCCCCCCCCCCCCCC(CCCCCCCCCCCCCCCC)C(=O)OC[C@H](COP(=O)([O-])OCC[N+](C)(C)C)OC(=O)C(CCCCCCCCCCCCCCCC)CCCCCCCCCCCCCCCC] |
| Lipid-11 | 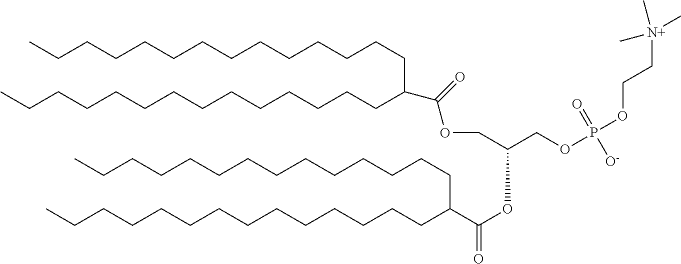 [SMILES: CCCCCCCCCCCCCCC(CCCCCCCCCCCCCC)C(=O)OC[C@H](COP(=O)([O-])OCC[N+](C)(C)C)OC(=O)C(CCCCCCCCCCCCCC)CCCCCCCCCCCCCC] |
| Lipid-12 | 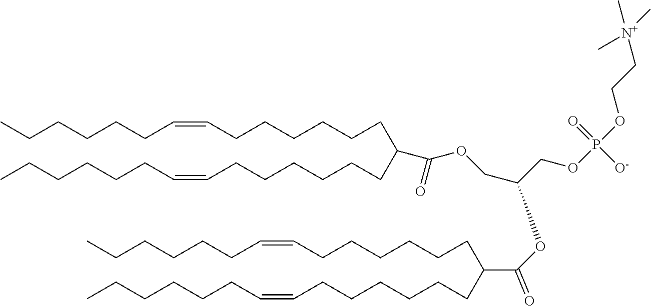 [SMILES: CCCCCC/C=C\CCCCCCC(CCCCCC/C=C\CCCCCC)C(=O)OC[C@H](COP(=O)([O-])OCC[N+](C)(C)C)OC(=O)C(CCCCCC/C=C\CCCCCC)CCCCCC/C=C\CCCCCC] |
| Lipid-13 | 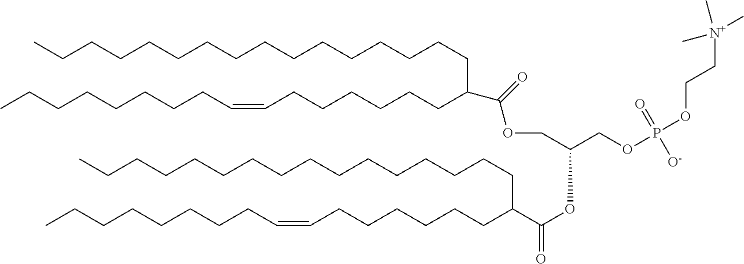 [SMILES: CCCCCCCC/C=C\CCCCCCC(CCCCCCCCCCCCCCCC)C(=O)OC[C@H](COP(=O)([O-])OCC[N+](C)(C)C)OC(=O)C(CCCCCC/C=C\CCCCCCCC)CCCCCCCCCCCCCCCC] |
| Lipid-14 | 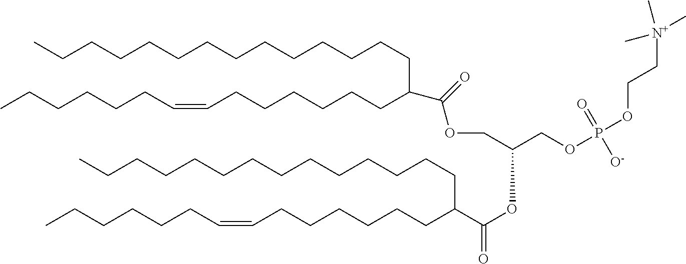 [SMILES: CCCCCC/C=C\CCCCCCC(CCCCCCCCCCCCCC)C(=O)OC[C@H](COP(=O)([O-])OCC[N+](C)(C)C)OC(=O)C(CCCCCC/C=C\CCCCCC)CCCCCCCCCCCCCC] |
| Lipid-15 | 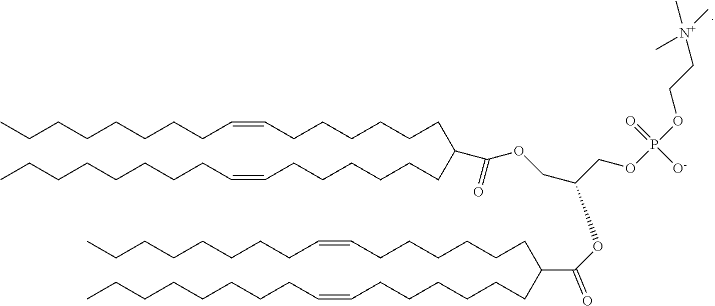 [SMILES: CCCCCCCC/C=C\CCCCCCC(CCCCCC/C=C\CCCCCCCC)C(=O)OC[C@H](COP(=O)([O-])OCC[N+](C)(C)C)OC(=O)C(CCCCCC/C=C\CCCCCCCC)CCCCCC/C=C\CCCCCCCC] |

12. A lipid nanoparticle formulation comprising a compound according to claim 1, and optionally a PEG-modified lipid.

13. A lipid nanoparticle formulation comprising a compound according to claim 1 and optionally a cholesterol.

14. A lipid nanoparticle comprising a compound according to claim 1, a PEG-modified lipid, a non-cationic lipid and cholesterol.

15. The formulation according to claim 13 wherein said PEG-modified lipid is DMG-PEG.

16. The formulation according to claim 13, wherein said non-cationic lipid is selected from distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), phosphatidylserine, sphingolipids, cerebrosides, gangliosides, 16-O-monomethyl PE, 16-O-dimethyl PE, 18-1-trans PE, 1-stearoyl-2-oleoyl-phosphatidyethanolamine (SOPE), or a mixture thereof.

17. The formulation according to claim 13, wherein said non-cationic lipid is DOPE or DSPC.

18. A composition comprising a messenger RNA (mRNA) encoding a protein or a peptide, encapsulated within a lipid nanoparticle comprising a compound according to claim 1, a PEG-modified lipid and a non-cationic lipid.

19. The composition according to claim 17, wherein said lipid nanoparticle has a size less than about 150 nm or about 100 nm.

20. The composition according to claim 17, wherein said mRNA is Cas9 mRNA.

21. A method for delivery of mRNA for in vivo production of a protein or a peptide comprising administering to a subject a composition comprising an mRNA that encodes the protein or the peptide, wherein the mRNA is encapsulated within a lipid nanoparticle and wherein the administering of the composition results in the expression of the protein or the peptide encoded by the mRNA, wherein the lipid nanoparticle comprises, a PEG-modified lipid, a compound according to claim 1, a non-cationic lipid and cholesterol.

22. The method according to claim 20, wherein the non-cationic lipid is selected from DOPE and DSPC.

23. The method according to claim 20, wherein said mRNA is Cas9 mRNA.

24. A process of encapsulating mRNA in lipid nanoparticles comprising a step of mixing (a) an mRNA solution comprising one or more mRNAs with (b) a lipid solution comprising one or more compounds of claim 1, one or more non-cationic lipids, and one or more PEG-modified lipids.
